(19) United States
(12) Patent Application Publication
(10) Pub. No.: US 2023/0270870 A1
(43) Pub. Date: Aug. 31, 2023

(54) DOSAGE OF AN ANTIBODY-DRUG CONJUGATE

(71) Applicant: DAIICHI SANKYO COMPANY, LIMITED, Tokyo (JP)

(72) Inventors: Yutaka NOGUCHI, Tokyo (JP); Tomonari YAMASHITA, Tokyo (JP); Daisuke OKAJIMA, Tokyo (JP); Takuma IGUCHI, Tokyo (JP); Satoru YASUDA, Tokyo (JP); Jonathan GREENBERG, Basking Ridge, NJ (US)

(73) Assignee: DAIICHI SANKYO COMPANY, LIMITED, Tokyo (JP)

(21) Appl. No.: 17/612,765

(22) PCT Filed: May 28, 2020
(86) PCT No.: PCT/IB2020/055078

§ 371 Date: Nov. 19, 2021

Related U.S. Application Data
(60) Provisional application No. 62/853,970, filed on May 29, 2019.
(60) Provisional application No. 62/896,478, filed on Sep. 5, 2019.

(51) Int. Cl. A61K 47/68; A61K 47/54; A61K 47/65; A61P 35/00

(52) U.S. Cl. CPC A61K 47/6803; A61K 47/65; A61K 47/545; A61K 47/6851; A61P 35/00

(57) ABSTRACT

The present disclosure relates to the field of pharmaceutical preparations, dosage regimens, and administration of an antibody-drug conjugate (ADC). More specifically, the ADC is composed of an anti-trophoblast cell surface antigen 2 (TROP2) antibody connected via a linker to an anticancer agent, such as topoisomerase I inhibitor.

### FIELD OF THE INVENTION

The present disclosure relates to the field of pharmaceutical preparations, dosage regimens, and administration of an antibody-drug conjugate (ADC). More specifically, the ADC is composed of an anti-trophoblast cell surface antigen 2 (TROP2) antibody connected via a linker to a topoisomerase I inhibitor, such as a derivative of exatecan.

### RELATED APPLICATIONS

This application claims priority under 35 U.S.C. § 119(e) to U.S. Provisional Application No. 62/853,970 filed May 29, 2019, and U.S. Provisional Application No. 62/896,478 filed Sep. 5, 2019, the entire contents of which are incorporated herein by reference.

### BACKGROUND

The following discussion is merely provided to aid the reader in understanding the disclosure and is not admitted to describe or constitute prior art thereto.

Trophoblast cell surface antigen 2 (TROP2) is a 323 amino acid transmembrane glycoprotein encoded by the Tacstd2 gene. It is an intracellular calcium signal transducer (Ripani E, et al., Int. J. Cancer, 76(5), 671-676 (1998), and El Sewedy T, et al., Int. J. Cancer, 75(2), 324-330 (1998)) that is differentially expressed in many cancers. It signals cells for self-renewal, proliferation, invasion, and survival. TROP2 is additionally involved in immune resistance, which is common to human trophoblasts and cancer cells (Faulk WP, et al., Proc. Natl. Acad. Sci.75(4), 1947-1951 (1978), and Lipinski M, et al., Proc. Natl. Acad. Sci. 78(8), 5147-5150 (1981)). The DNA sequence and amino acid sequence of human TROP2 are available on public databases, for example, under Accession Nos. NM_002353 and NP_002344 (NCBI).

TROP2 was found to be overexpressed in various epithelial cell carcinomas compared to a low level of expression in normal epithelial cells. The expression of TROP2 was also reported to correlate with the poor prognosis of colorectal cancer (Ohmachi T, et al., Clin. Cancer Res., 12(10), 3057-3063 (2006)), gastric cancer (Muhlmann G, et al., J. Clin. Pathol., 62(2), 152-158 (2009)), pancreatic cancer (Fong D, et al., Br. J. Cancer, 99(8), 1290-1295 (2008)), oral cancer (Fong D, et al., Mod. Pathol., 21(2), 186-191 (2008)), and glioma (Ning S, et al., Neurol. Sci., 34(10), 1745-1750 (2013)), among other. Using colorectal cancer cells as a model, it was further reported that the expression of TROP2 is involved in scaffold-independent cell growth of tumor cells and tumorigenesis in immunodeficient mice (Wang J, et al., Mol. Cancer Ther., 7(2), 280-285 (2008)).

Given TROP2's association with various types of cancer, a plurality of anti-TROP2 antibodies have been prepared and studied. Among these antibodies, there have been reports of an unconjugated antibody that exhibits some antitumor activity in nude mouse xenograft models (International Patent Publication Nos. WO2008/144891, WO2011/145744, WO2011/155579, and WO2013/077458) as well as an antibody that exhibits antitumor activity as an antibody-drug conjugate (ADC) (International Patent Publication Nos. WO2003/074566, WO2011/068845, and WO2013/068946, and U.S. Pat. No. 7999083). However, the strength and coverage of anti-TROP2 antibodies and ADCs has been insufficient to date, and there is still an unsatisfied medical need to utilize TROP2 as a therapeutic target.

The present disclosure provides a TROP2-specific ADC and dosage regimens for the same to treat various cancer. Accordingly, the present disclosure fulfills the need in the art for safe and effective cancer treatments that target TROP2.

### SUMMARY

Antitumor antibodies targeting TROP2 have been unsuccessful to date, and many antitumor low-molecular-weight compounds have a problem with safety due to unacceptable side effects and toxicity (even with compounds that have an excellent antitumor effect). Accordingly, there remains a need to achieve superior therapeutic effect while concurrently enhancing the safety. Thus, an object of the present disclosure is to provide an antitumor drug with excellent therapeutic efficacy and safety.

When the antitumor compound exatecan is converted into an antibody-drug conjugate, via a linker structure moiety, by conjugation to an anti-TROP2 antibody that is capable of targeting tumor cells, recognizing tumor cells, binding to tumor cells, internalizing within tumor cells, or the like, the cytocidal activity based on the antibody can be acquired, and the antitumor compound can be more surely delivered to tumor cells to specifically exhibit the antitumor effect. Thus, the antitumor effect can be surely exhibited, and a dose of the antitumor compound can be reduced compared to administering the compound alone, which reduces the negative side effects on normal cells and increases safety.

Described herein are novel TROP2-targeting ADC comprising an exatecan derivative and an anti-TROP2 antibody, and methods of using the same.

In one aspect, the present disclosure provides an anti-TROP2 antibody-drug conjugate for use in treating or preventing cancer, the antibody-drug conjugate comprising an anti-TROP2 antibody and an antitumor compound connected by a linker.

In another aspect, the present disclosure provides a method of treating or preventing cancer in a subject, comprising administering to a subject with cancer an anti-TROP2 antibody-drug conjugate comprising an anti-TROP2 antibody and an antitumor compound connected by a linker.

In another aspect, the present disclosure provides a use of an anti-TROP2 antibody-drug conjugate in the manufacture of a medicament for treating or preventing cancer, the antibody-drug conjugate comprising an anti-TROP2 antibody and an antitumor compound connected by a linker.

In some embodiments, the linker and the antitumor compound are represented by the following formula:
-(Succinimid-3-yl-N)—CH₂CH₂CH₂CH₂CH₂—C(═O)—GGFG—NH—CH₂—O—CH₂—C(═O)—(NH—DX) wherein -(Succinimid-3-yl-N)- has a structure represented by the following formula:

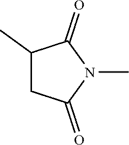

which is connected to the antibody at position 3 thereof and is connected to a methylene group in the linker structure containing this structure on the nitrogen atom at position 1, and (NH-DX) represents a group represented by the following formula:

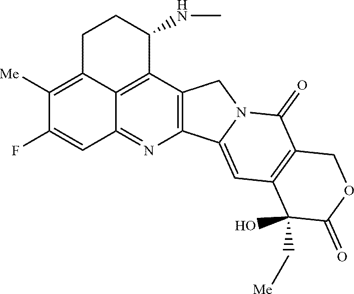 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is the connecting position.

In some embodiments, the anti-TROP2 antibody comprises CDRH1 consisting of the amino acid sequence of SEQ ID NO: 23, CDRH2 consisting of the amino acid sequence of SEQ ID NO: 24 and CDRH3 consisting of the amino acid sequence of SEQ ID NO: 25 in its heavy chain variable region and CDRL1 consisting of the amino acid sequence of SEQ ID NO: 26, CDRL2 consisting of the amino acid sequence of SEQ ID NO: 27 and CDRL3 consisting of the amino acid sequence of SEQ ID NO: 28 in its light chain variable region.

In some embodiments, an average number of units of the antitumor compound conjugated per antibody is in a range of from 2 to 8 or 3 to 8. In some embodiments, an average number of units of the antitumor compound conjugated per antibody is in a range of 3.4 to 4.5. In some embodiments, an average number of units of the antitumor compound conjugated per antibody is 4.

In some embodiments, the antibody comprises a heavy chain variable region comprising amino acids 1-121 of SEQ ID NO: 45 and a light chain variable region comprising amino acids 1-109 of SEQ ID NO: 46. In some embodiments, the antibody comprises a heavy chain comprising SEQ ID NO: 45 and a light chain comprising SEQ ID NO: 46. In some embodiments, the anti-TROP2 antibody lacks a lysine residue at the carboxyl terminus of the heavy chain.

In some embodiments, a dose of the antibody-drug conjugate is in a range of 2 mg/kg to 10 mg/kg is administered to a subject with cancer. In some embodiments, a dose of the antibody-drug conjugate of about 4 mg/kg is administered to a subject with cancer. In some embodiments, a dose of the antibody-drug conjugate of about 6 mg/kg is administered to a subject with cancer. In some embodiments, a dose of the antibody-drug conjugate of about 8 mg/kg is administered to a subject with cancer.

In some embodiments, the antibody-drug conjugate is administered by intravenous administration.

In some embodiments, the antibody-drug conjugate is administered once every 3 weeks or once every 4 weeks.

In some embodiments, the cancer is selected from the group consisting of lung cancer, kidney cancer, urothelial cancer, colorectal cancer, prostate cancer, glioblastoma multiforme, ovarian cancer, pancreatic cancer, breast cancer, melanoma, liver cancer, bladder cancer, gastric cancer, cervical cancer, head and neck cancer, and esophageal cancer. In some embodiments, the lung cancer is non-small cell lung cancer (NSCLC).

In some embodiments, the cancer is resistant or refractory. In some embodiments, the resistance or refractoriness is resistance or refractoriness acquired by the cancer due to treatment with an anticancer drug. In some embodiments, the anticancer drug is an EGFR-inhibitor, an ALK-inhibitor, a platinum-based chemotherapeutic agent, or a checkpoint inhibitor. In some embodiments, the anticancer drug is gefitinib, erlotinib, osimertinib, affatinib, alectinib, crizotinib, ceritinib, cisplatin, carboplatin, nivolumab, pembrolizumab, atezolizumab, avelumab, ipilimumab, durvalumab, tislelizumab, sintilimab, or cemiplimab.

In some embodiments, the cancer is a TROP2-expressing caner. In some embodiments, the TROP2-expressing cancer is TROP2-overexpressing cancer. In some embodiments, the TROP2-overexpressing cancer is cancer given a high score for the expression of TROP2 in an immunohistochemical method. In some embodiments, the TROP2-overexpressing cancer is cancer given a high score for the expression of TROP2 in an in situ hybridization method.

In some embodiments, the cancer is an inoperable or recurrent cancer.

Also provided herein are pharmaceutical compositions containing the antibody-drug conjugate according to any one of the foregoing aspects or embodiments or a salt thereof as an active component, and a pharmaceutically acceptable formulation component.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows the structure of a TROP2-targeting antibody-drug conjugate (herein referred to as “antibody drug conjugate (1)”) with a topoisomerase I inhibitor (DXd). The ADC possesses a tetrapeptide linker bound to a cysteine residue on the antibody. The pictured ADC has a drug-to-antibody ratio of 4:1 (i.e., DAR4).

FIG. 2 shows the heavy and light chain sequences of an anti-TROP2 antibody that can be incorporated into the disclosed ADC and a graphical formula of the cytotoxic agent linked to the antibody.

FIG. 3 shows the antitumor effects of antibody-drug conjugates (1) and (2) in a murine xenograft CFPAC-1 tumor model.

FIG. 4 shows an estimation of plasma concentration during repeated administration of DS-1062a in humans.

FIG. 5 shows a Phase 1 study design for treating non-small cell lung cancer (NSCLC) patients.

FIG. 6 shows patient demographics and baseline characteristics for the initial Phase 1 study (Example 5).

FIG. 7 shows the number of patients in the initial Phase 1 study (Example 5) with treatment-emergent adverse events (TEAEs), which occurred in ≥10% of patients, regardless of causality.

FIG. 8 shows the tumor response of the subjects (N=35) in the initial Phase 1 study (Example 5).

FIG. 9 shows the response of tumors in target (A, B, and C) and non-target (D) lesions after DS-1062a treatment in the initial Phase 1 study (Example 5). Panel A shows a reduction in the size of a target lesion in a patients treated with 4.0 mg/kg of DS-1062a. Panel B shows a reduction in the size of a target lesion in another patient treated with 4.0 mg/kg of DS-1062a. Panel C shows a reduction in the size of target lesions in a patients treated with 2.0 mg/kg of DS-1062a. Panel D shows a decrease in a number of non-target lesions in the same patients as Panel C.

FIG. 10 shows change in tumor size of the subject in the initial Phase 1 study (Example 5). The top panel shows the best percentage change in sum of longest dimension measures from baseline in target lesions of subjects from the initial Phase 1 study (Example 5). The bottom panels shows a spider plot of the tumor size change separated by dosing group.

FIG. 11 shows mean plasma concentrations of DS-1062a in cycle 1 (PK analysis set).

FIG. 12 shows a summary of efficacy demonstrated by the initial Phase 1 study (Example 5).

FIG. 13 shows the number of patients in the Phase 1 study as of the new cut-off date (Example 6) with treatment-emergent adverse events (TEAEs), regardless of causality.

FIG. 14 shows the best percentage change in sum of longest dimension measures from baseline in target lesions of subjects from the Phase 1 study as of the new cut-off date (Example 6).

FIG. 15 shows a clear dose-effect on frequency of response by illustrating the percent change in tumor size for each dosing group over the course of the Phase 1 study as of the new cut-off date (Example 6).

FIG. 16 shows the durable antitumor responses seen across multiple dose levels. Many patients saw a partial response (PR) or stable disease (SD). Only two patients had progressive disease (PD) at the close of the study (Example 6).

FIG. 17 shows TROP2 immunohistochemistry H score (IHC) based on pretreatment biopsies from patients in the Phase 1 study as of the new cut-off date (Example 6). IHC scores tended to be higher in those patients that achieved positive results such as partial response (PR). For the purposes of these figures the following abbreviations were used: anaplastic lymphoma kinase inhibitor (ALKi), baseline (BL), cycle 3 day 1 (C3D1), circulating free DNA (cfDNA), epidermal growth factor receptor inhibitor (EGFRi), end of treatment (EOT), human epidermal growth factor receptor 2 inhibitor (HER2i), immunohistochemistry (IHC), histo score (H-score), immune-oncology (I/O), non-evaluable (NE), partial response (PR), progressive disease (PD), stable disease (SD), patient (Pt), variant allele frequency (VAF).

FIG. 18 show the results from preclinical studies showing that antibody drug conjugate (1) possessed antitumor activity in lung cancer xenograft mouse models with stronger antitumor activity in TROP2-positive tumors (NCI-H2170 and HCC827) as opposed to TROP2-negative tumors (Calu-6).

FIG. 19 shows changes in variable allele frequency based on cell free DNA (cfDNA) over the course of treatment. The results indicate that cfDNA generally decreased as a result of treatment.

FIG. 20 shows the overall response rate (ORR) as assessed by change in tumor volume of the subjects in the various dosing groups of the Phase 1 study as of the new cut-off date (Example 6).

FIG. 21 shows a summary of efficacy demonstrated by the Phase 1 study as of the new cut-off date (Example 6).

FIG. 22 shows a spider plot of tumor size change by dose group from the preliminary efficacy study (Example 7).

FIG. 23 shows the plasma concentration of antibody-drug conjugate (1), total antibody, and free drug (payload) as determined by pharmacokinetic measurements from the preliminary efficacy study (Example 7).

### DETAILED DESCRIPTION

Hereinafter, various embodiments of the novel TROP2-targeting ADC and methods of using the same will be described with reference to the drawings. The embodiments described below are given as typical examples of the embodiments of the present invention and are not intended to limit the scope of the present invention.

The anti-TROP2 antibody-drug conjugate of the present invention is an antitumor drug in which an anti-TROP2 antibody is conjugated to an antitumor compound via a linker structure moiety and explained in detail below.

### Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used in the specification and claims, the singular form “a,” “an” and “the” include singular and plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

The terms “individual,” “subject,” and “patient” are used interchangeably herein, and refer to any individual mammal, e.g., bovine, canine, feline, equine, simian, porcine, camelid, bat, or human, being treated according to the disclosed methods or uses. In preferred embodiments, the subject is a human.

As used herein, the phrases “effective amount,” “therapeutically effective amount,” and “therapeutic level” mean the dosage or concentration in a subject that provides the specific pharmacological effect for which the ADC is administered in a subject in need of such treatment, i.e. to treat or prevent a cancer (e.g., a lung cancer, TROP2-expressing cancer, or a resistant or refractory cancer). It is emphasized that a therapeutically effective amount or therapeutic level of an ADC will not always be effective in treating the cancers described herein, even though such dosage is deemed to be a therapeutically effective amount by those of skill in the art. For convenience only, exemplary dosages, drug delivery amounts, therapeutically effective amounts, and therapeutic levels are provided below. Those skilled in the art can adjust such the amount in accordance with standard practices as needed to treat a specific subject and/or condition. The therapeutically effective amount may vary based on the route of administration and dosage form, the age and weight of the subject, and/or the subject’s condition, including the type and severity of the cancer.

The terms “treatment” or “treating” as used herein with reference to a cancer refer to reducing, suppressing, or eliminating the cancer; reducing, suppressing, or eliminating cancer cell growth; reducing, suppressing, or eliminating spread of the cancer; or causing a tumor or metastasis to regress or die. Treatment and treating may also, optionally, mean improving quality or life or overall survival of a subject, even if cancer cell growth is not inhibited and/or the cancer does not die.

The terms “prevent” or “preventing” as used herein with reference to a cancer refer to precluding or preventing the occurrence of metastasis (i.e., growth of cancer in secondary sites where the cancer is not present at the commencement of treatment), as well as precluding or preventing recurrence of a cancer if a subject achieves remission or a cancer/tumor is completely destroyed or killed.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington’s Pharmaceutical Sciences, 15th Ed., Mack Publ. Co., Easton, PA [1975].

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient’s system and, thus, is subject to metabolism and other like processes, for example, subcutaneous administration.

The term “gene” as used herein includes not only DNA, but also mRNA thereof, cDNA thereof, and cRNA thereof.

The term “polynucleotide” as used herein is used with the same meaning as a nucleic acid and also includes DNA, RNA, probes, oligonucleotides, and primers.

The terms “polypeptide” and “protein” as used herein are used without distinction.

The term “cell” as used herein also includes cells in an animal individual and cultured cells.

The term “TROP2” as used herein is used in the same meaning as TROP2 protein.

The term “CDR” as used herein refers to a complementarity determining region (CDR). It is known that each heavy and light chain of an antibody molecule has three complementarity determining regions (CDRs). The CDR is also called the hypervariable domain, and is present in a variable region of each heavy and light chain of an antibody. It is a site which has unusually high variability in its primary structure, and there are three separate CDRs in the primary structure of each heavy and light polypeptide chain. In this specification, as for the CDRs of an antibody, the CDRs of the heavy chain are represented by CDRH1, CDRH2, and CDRH3 from the amino-terminal side of the amino acid sequence of the heavy chain, and the CDRs of the light chain are represented by CDRL1, CDRL2, and CDRL3 from the amino-terminal side of the amino acid sequence of the light chain. These sites are proximate to one another in the tertiary structure and determine the specificity for an antigen to which the antibody binds.

The phrase “hybridization is performed under stringent conditions” as used herein refers to a process in which hybridization is performed under conditions under which identification can be achieved by performing hybridization at 68° C. in a commercially available hybridization solution ExpressHyb Hybridization Solution (manufactured by Clontech, Inc.) or by performing hybridization at 68° C. in the presence of 0.7 to 1.0 M NaCl using a filter having DNA immobilized thereon, followed by performing washing at 68° C. using 0.1 to 2 x SSC solution (1 x SSC solution is composed of 150 mM NaCl and 15 mM sodium citrate) or under conditions equivalent thereto.

The term “several” as used herein refers to 1 to 10, 1 to 9, 1 to 8, 1 to 7, 1 to 6, 1 to 5, 1 to 4, 1 to 3, or 1 to 2.

As the amino acid substitution in this specification, a conservative amino acid substitution is preferred. The conservative amino acid substitution refers to a substitution occurring within a group of amino acids related to amino acid side chains. Preferred amino acid groups are as follows: an acidic group (aspartic acid and glutamic acid); a basic group (lysine, arginine, and histidine); a non-polar group (alanine, valine, leucine, isoleucine, proline, phenylalanine, methionine, and tryptophan); and an uncharged polar family (glycine, asparagine, glutamine, cysteine, serine, threonine, and tyrosine). More preferred amino acid groups are as follows: an aliphatic hydroxyl group (serine and threonine); an amide-containing group (asparagine and glutamine); an aliphatic group (alanine, valine, leucine, and isoleucine); and an aromatic group (phenylalanine, tryptophan, and tyrosine). Such an amino acid substitution is preferably performed within a range which does not impair the properties of a substance having the original amino acid sequence.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### Trop2

TROP2 is a member of the TACSTD family expressed in human trophoblasts and is a single-pass transmembrane type 1 cell membrane protein involved in immune resistance, which is common to human trophoblasts and cancer cells.

For the purposes of the present disclosure, TROP2 protein can be directly purified from the TROP2-expressing cells of a human or a non-human mammal (such as a rat or a mouse) and used, or a cell membrane fraction of the above-described cells can be prepared and used. Further, TROP2 can be obtained by in vitro synthesis thereof or production thereof in a host cell through genetic engineering. In the genetic engineering, specifically, after TROP2 cDNA is integrated into a vector capable of expressing TROP2 cDNA, the TROP2 protein can be obtained by synthesizing it in a solution containing an enzyme, a substrate and an energy substance required for transcription and translation, or by expressing TROP2 in another prokaryotic or eukaryotic transformed host cell. Alternatively, the above-described genetically engineered TROP2-expressing cells or a cell line expressing TROP2 may be used as the TROP2 protein.

The DNA sequence and amino acid sequence of TROP2 are available on a public database and can be referred to, for example, under Accession Nos. NM_002353 and NP_002344 (NCBI).

Further, a protein which consists of an amino acid sequence wherein one or several amino acids are substituted, deleted and/or added in any of the above-described amino acid sequences of TROP2 and also has a biological activity equivalent to that of the protein is also included in TROP2.

The human TROP2 protein comprises a signal sequence consisting of N-terminal 26 amino acid residues, an extracellular domain consisting of 248 amino acid residues, a transmembrane domain consisting of 23 amino acid residues, and an intracellular domain consisting of 26 amino acid residues.

### Anti-TROP2 Antibody

The anti-TROP2 antibody used in the anti-TROP2 antibody-drug conjugate of the present disclosure may be derived from any species, and preferred examples of the species can include humans, rats, mice, and rabbits. In case when derived from other than human species, it is preferably chimerized or humanized using a well-known technique. The antibody of the present invention may be a polyclonal antibody or a monoclonal antibody and is preferably a monoclonal antibody.

The anti-TROP2 antibody is capable of targeting tumor cells, capable of recognizing a tumor cell, capable of binding to a tumor cell, capable of internalizing in a tumor cell, or the like, and can be converted into an antibody-drug conjugate by conjugation via a linker to a compound having antitumor activity.

The binding activity of the antibody against tumor cells can be confirmed using flow cytometry. Examples of the method for confirming the internalization of the antibody into tumor cells can include (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282, December 2004), or (3) a Mab-ZAP assay using an immunotoxin binding to the therapeutic antibody wherein the toxin is released upon incorporation into cells to inhibit cell growth (Bio Techniques 28: 162-165, January 2000). A recombinant complex protein of a catalytic region of diphtheria toxin and protein G may be used as the immunotoxin.

Because the drug conjugated in the antibody-drug conjugate exerts an antitumor effect, it is preferred but not essential that the antibody itself should have an antitumor effect. For the purpose of specifically and selectively exerting the cytocidal activity of the antitumor compound on tumor cells, it is important and also preferred that the antibody should have the property of internalizing to migrate into tumor cells.

The anti-TROP2 antibody can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen are fused with myeloma cells according to a method known in the art (e.g., Kohler and Milstein, Nature (1975) 256, p. 495-497; and Kennet, R. ed., Monoclonal Antibodies, p. 365-367, Plenum Press, N.Y. (1980)) to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can be also obtained using a method of immunizing animals with the above-described genetically engineered antigen-expressing cells or a cell line expressing the antigen.

The anti-TROP2 antibody can obtained by a procedure known in the art.

The anti-TROP2 antibody that can be used in the present invention is not particularly limited, and, for example, those specified by the amino acid sequences shown in the Sequence Listing of the present application can be preferably used. The anti-TROP2 antibody used in the present invention preferably has properties as described below.

An antibody having the following properties:
(a) specifically binding to TROP2, and
(b) having an activity of internalizing in TROP2-expressing cells by binding to TROP2.

The antibody according to (1), wherein TROP2 is human TROP2.

The antibody according to (1) or (2), wherein the antibody has a complementarity determining region (CDR) H1, CDRH2, and CDRH3 of a heavy chain of SEQ ID NO: 45, and/or a CDRL1, CDRL2, and CDRL3 of a light chain of SEQ ID NO: 46. Alternatively or additionally, the antibody according to (1) or (2), wherein the antibody has CDRH1 comprising the amino acid sequence represented by SEQ ID NO: 23, CDRH2 comprising the amino acid sequence represented by SEQ ID NO: 24, and CDRH3 comprising the amino acid sequence represented by SEQ ID NO: 25 as heavy chain complementarity determining regions, and CDRL1 comprising the amino acid sequence represented by SEQ ID NO: 26, CDRL2 comprising the amino acid sequence represented by SEQ ID NO: 27, and CDRL3 comprising the amino acid sequence represented by SEQ ID NO: 28 as light chain complementarity determining regions.

The antibody according to any of (1) to (3), wherein the constant region thereof is a human-derived constant region.

The antibody according to any of (1) to (4), wherein the antibody is a humanized antibody.

The antibody according to (5), wherein the antibody has a heavy chain variable region comprising an amino acid sequence selected from the group consisting of (a) an amino acid sequence described in amino acid positions 1 to 121 in SEQ ID NO: 45, (b) an amino acid sequence having at least 95% or higher homology to (a), and (c) an amino acid sequence derived from any of the sequences (a) or (b) by the deletions, replacements, or additions of at least one amino acid, and a light chain variable region comprising an amino acid sequence selected from the group consisting of (d) an amino acid sequence described in amino acid positions 1 to 109 in SEQ ID NO: 46, (e) an amino acid sequence having at least 95% or higher homology to (d) and (f) an amino acid sequence derived from any of the sequences (d) or (e) by the deletions, replacements, or additions of at least one amino acid. Alternatively or additionally, the antibody according to (5), wherein the antibody has a heavy chain variable region comprising an amino acid sequence selected from the group consisting of (a) an amino acid sequence described in amino acid positions 20 to 140 in SEQ ID NO: 12, (b) an amino acid sequence described in amino acid positions 20 to 140 in SEQ ID NO: 14, (c) an amino acid sequence described in amino acid positions 20 to 140 in SEQ ID NO: 16, (d) an amino acid sequence having at least 95% or higher homology to any of the sequences (a) to (c), and (e) an amino acid sequence derived from any of the sequences (a) to (c) by the deletions, replacements, or additions of at least one amino acid, and a light chain variable region comprising an amino acid sequence selected from the group consisting of (f) an amino acid sequence described in amino acid positions 21 to 129 in SEQ ID NO: 18, (g) an amino acid sequence described in amino acid positions 21 to 129 in SEQ ID NO: 20, (h) an amino acid sequence described in amino acid positions 21 to 129 in SEQ ID NO: 22, (i) an amino acid sequence having at least 95% or higher homology to any of the sequences (f) to (h), and (j) an amino acid sequence derived from any of the sequences (f) to (h) by the deletions, replacements, or additions of at least one amino acid.

The antibody according to (6), wherein the antibody has a heavy chain variable region comprising an amino acid sequence described in amino acid positions 1 to 121 in SEQ ID NO: 45 and a light chain variable region comprising an amino acid sequence described in amino acid positions 1 to 109 in SEQ ID NO: 46. Alternatively or additionally, the antibody according to (6), wherein the antibody has a heavy chain variable region and a light chain variable region selected from the group consisting of a heavy chain variable region comprising an amino acid sequence described in amino acid positions 20 to 140 in SEQ ID NO: 12 and a light chain variable region comprising an amino acid sequence described in amino acid positions 21 to 129 in SEQ ID NO: 18, a heavy chain variable region comprising an amino acid sequence described in amino acid positions 20 to 140 in SEQ ID NO: 12 and a light chain variable region comprising an amino acid sequence described in amino acid positions 21 to 129 in SEQ ID NO: 20, a heavy chain variable region comprising an amino acid sequence described in amino acid positions 20 to 140 in SEQ ID NO: 12 and a light chain variable region comprising an amino acid sequence described in amino acid positions 21 to 129 in SEQ ID NO: 22, a heavy chain variable region comprising an amino acid sequence described in amino acid positions 20 to 140 in SEQ ID NO: 14 and a light chain variable region comprising an amino acid sequence described in amino acid positions 21 to 129 in SEQ ID NO: 18, a heavy chain variable region comprising an amino acid sequence described in amino acid positions 20 to 140 in SEQ ID NO: 14 and a light chain variable region comprising an amino acid sequence described in amino acid positions 21 to 129 in SEQ ID NO: 20, a heavy chain variable region comprising an amino acid sequence described in amino acid positions 20 to 140 in SEQ ID NO: 14 and a light chain variable region comprising an amino acid sequence described in amino acid positions 21 to 129 in SEQ ID NO: 22, a heavy chain variable region comprising an amino acid sequence described in amino acid positions 20 to 140 in SEQ ID NO: 16 and a light chain variable region comprising an amino acid sequence described in amino acid positions 21 to 129 in SEQ ID NO: 18, a heavy chain variable region comprising an amino acid sequence described in amino acid positions 20 to 140 in SEQ ID NO: 16 and a light chain variable region comprising an amino acid sequence described in amino acid positions 21 to 129 in SEQ ID NO: 20, and a heavy chain variable region comprising an amino acid sequence described in amino acid positions 20 to 140 in SEQ ID NO: 16 and a light chain variable region comprising an amino acid sequence described in amino acid positions 21 to 129 in SEQ ID NO: 22.

The antibody according to (7), wherein the antibody has a heavy chain variable region and a light chain variable region selected from the group consisting of a heavy chain variable region comprising an amino acid sequence described in amino acid positions 20 to 140 in SEQ ID NO: 12 and a light chain variable region comprising an amino acid sequence described in amino acid positions 21 to 129 in SEQ ID NO: 18, a heavy chain variable region comprising an amino acid sequence described in amino acid positions 20 to 140 in SEQ ID NO: 14 and a light chain variable region comprising an amino acid sequence described in amino acid positions 21 to 129 in SEQ ID NO: 18, a heavy chain variable region comprising an amino acid sequence described in amino acid positions 20 to 140 in SEQ ID NO: 14 and a light chain variable region comprising an amino acid sequence described in amino acid positions 21 to 129 in SEQ ID NO: 20, and a heavy chain variable region comprising an amino acid sequence described in amino acid positions 20 to 140 in SEQ ID NO: 16 and a light chain variable region comprising an amino acid sequence described in amino acid positions 21 to 129 in SEQ ID NO: 22.

The antibody according to (6) or (7), wherein the antibody comprises a heavy chain comprising an amino acid sequence described in amino acid positions 1 to 451 in SEQ ID NO: 45 and a light chain comprising an amino acid sequence described in amino acid positions 1 to 214 in SEQ ID NO: 46. Alternatively or additionally, the antibody according to (6) or (7), wherein the antibody comprises a heavy chain and a light chain selected from the group consisting of a heavy chain comprising an amino acid sequence described in amino acid positions 20 to 470 in SEQ ID NO: 12 and a light chain comprising an amino acid sequence described in amino acid positions 21 to 234 in SEQ ID NO: 18, a heavy chain comprising an amino acid sequence described in amino acid positions 20 to 470 in SEQ ID NO: 12 and a light chain comprising an amino acid sequence described in amino acid positions 21 to 234 in SEQ ID NO: 20, a heavy chain comprising an amino acid sequence described in amino acid positions 20 to 470 in SEQ ID NO: 12 and a light chain comprising an amino acid sequence described in amino acid positions 21 to 234 in SEQ ID NO: 22, a heavy chain comprising an amino acid sequence described in amino acid positions 20 to 470 in SEQ ID NO: 14 and a light chain comprising an amino acid sequence described in amino acid positions 21 to 234 in SEQ ID NO: 18, a heavy chain comprising an amino acid sequence described in amino acid positions 20 to 470 in SEQ ID NO: 14 and a light chain comprising an amino acid sequence described in amino acid positions 21 to 234 in SEQ ID NO: 20, a heavy chain comprising an amino acid sequence described in amino acid positions 20 to 470 in SEQ ID NO: 14 and a light chain comprising an amino acid sequence described in amino acid positions 21 to 234 in SEQ ID NO: 22, a heavy chain comprising an amino acid sequence described in amino acid positions 20 to 470 in SEQ ID NO: 16 and a light chain comprising an amino acid sequence described in amino acid positions 21 to 234 in SEQ ID NO: 18, a heavy chain comprising an amino acid sequence described in amino acid positions 20 to 470 in SEQ ID NO: 16 and a light chain comprising an amino acid sequence described in amino acid positions 21 to 234 in SEQ ID NO: 20, and a heavy chain comprising an amino acid sequence described in amino acid positions 20 to 470 in SEQ ID NO: 16 and a light chain comprising an amino acid sequence described in amino acid positions 21 to 234 in SEQ ID NO: 22.

The antibody according to (6) or (7), wherein the antibody comprises a heavy chain comprising the amino acid sequence represented by SEQ ID NO: 45 and a light chain comprising the amino acid sequence represented by SEQ ID NO: 46. Alternatively or additionally, the antibody according to (6) or (7), wherein the antibody comprises a heavy chain and a light chain selected from the group consisting of a heavy chain comprising the amino acid sequence represented by SEQ ID NO: 12 and a light chain comprising the amino acid sequence represented by SEQ ID NO: 18, a heavy chain comprising the amino acid sequence represented by SEQ ID NO: 12 and a light chain comprising the amino acid sequence represented by SEQ ID NO: 20, a heavy chain comprising the amino acid sequence represented by SEQ ID NO: 12 and a light chain comprising the amino acid sequence represented by SEQ ID NO: 22, a heavy chain comprising the amino acid sequence represented by SEQ ID NO: 14 and a light chain comprising the amino acid sequence represented by SEQ ID NO: 18, a heavy chain comprising the amino acid sequence represented by SEQ ID NO: 14 and a light chain comprising the amino acid sequence represented by SEQ ID NO: 20, a heavy chain comprising the amino acid sequence represented by SEQ ID NO: 14 and a light chain comprising the amino acid sequence represented by SEQ ID NO: 22, a heavy chain comprising the amino acid sequence represented by SEQ ID NO: 16 and a light chain comprising the amino acid sequence represented by SEQ ID NO: 18, a heavy chain comprising the amino acid sequence represented by SEQ ID NO: 16 and a light chain comprising the amino acid sequence represented by SEQ ID NO: 20, and a heavy chain comprising the amino acid sequence represented by SEQ ID NO: 16 and a light chain comprising the amino acid sequence represented by SEQ ID NO: 22.

The antibody according to (8), wherein the antibody comprises a heavy chain and a light chain selected from the group consisting of a heavy chain comprising an amino acid sequence described in amino acid positions 20 to 470 in SEQ ID NO: 12 and a light chain comprising an amino acid sequence described in amino acid positions 21 to 234 in SEQ ID NO: 18, a heavy chain comprising an amino acid sequence described in amino acid positions 20 to 470 in SEQ ID NO: 14 and a light chain comprising an amino acid sequence described in amino acid positions 21 to 234 in SEQ ID NO: 18, a heavy chain comprising an amino acid sequence described in amino acid positions 20 to 470 in SEQ ID NO: 14 and a light chain comprising an amino acid sequence described in amino acid positions 21 to 234 in SEQ ID NO: 20, and a heavy chain comprising an amino acid sequence described in amino acid positions 20 to 470 in SEQ ID NO: 16 and a light chain comprising an amino acid sequence described in amino acid positions 21 to 234 in SEQ ID NO: 22.

The antibody according to any of (1) to (11), wherein the antibody lacks a lysine residue at the carboxyl terminus of the heavy chain.

An antibody obtained by a method for producing the antibody according to any of (1) to (12), the method comprising the steps of: culturing a host cell transformed with an expression vector containing a polynucleotide encoding the antibody; and collecting the antibody of interest from the cultures obtained in the preceding step.

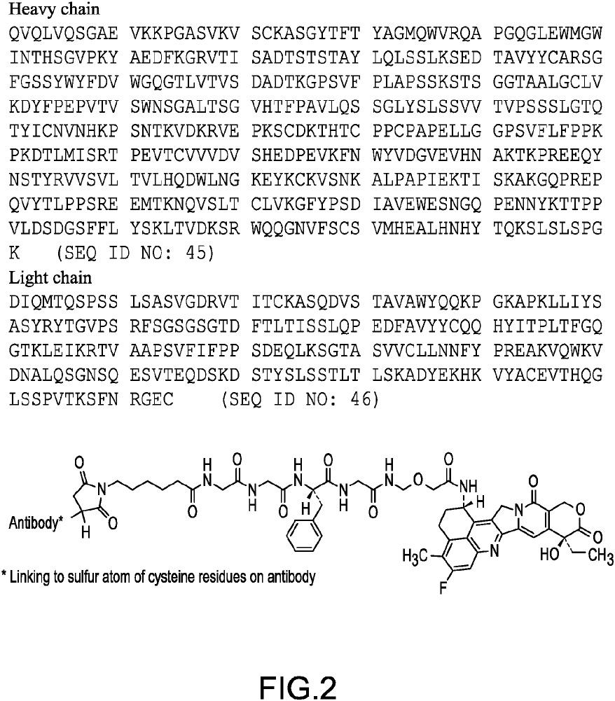

For the purposes of the present disclosure, the complete sequences of SEQ ID NOs: 45 and 46 are shown in Table 1 below (as well as in FIG. 2).

TABLE 1

Heavy and Light Chain Amino Acid Sequences of Exemplary Anti-TROP2 Antibody

| SEQ ID NO: | Amino Acid Sequence |
|---|---|
| 45 | QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK |
| 46 | DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC |

### Production of an Anti-TROP2 Antibody

An antibody against TROP2 of the invention can be obtained using a method usually carried out in the art, which involves immunizing an animal with TROP2 or an arbitrary polypeptide selected from the amino acid sequence of TROP2, and collecting and purifying the antibody produced in vivo. The biological species of TROP2 to be used as an antigen is not limited to being human, and an animal can be immunized with TROP2 derived from an animal other than humans such as a mouse or a rat. In this case, by examining the cross-reactivity between an antibody binding to the obtained heterologous TROP2 and human TROP2, an antibody applicable to a human disease can be selected.

Further, a monoclonal antibody can be obtained from a hybridoma established by fusing one or more antibody-producing cell(s) which produce an antibody against TROP2 with myeloma cells according to a known method (for example, Kohler and Milstein, Nature, (1975) 256, pp. 495-497; Kennet, R. ed., Monoclonal Antibodies, pp. 365-367, Plenum Press, N.Y. (1980)).

TROP2 to be used as an antigen can be obtained by expressing TROP2 gene in a host cell using genetic engineering. Specifically, a vector capable of expressing TROP2 gene can be produced, and the resulting vector can be transfected into a host cell to express the gene, and then, the expressed TROP2 can be purified.

Alternatively, the above-described genetically engineered TROP2-expressing cells or a cell line expressing TROP2 may be used as the TROP2 protein. Hereinafter, a method of obtaining an antibody against TROP2 is specifically described.

### Preparation of Antigen

Examples of the antigen to be used for producing the anti-TROP2 antibody include TROP2, or a polypeptide consisting of a partial amino acid sequence comprising at least 6 consecutive amino acids of TROP2, or a derivative obtained by adding a given amino acid sequence or carrier thereto.

TROP2 can be purified directly from human tumor tissues or tumor cells and used. Further, TROP2 can be obtained by synthesizing it in vitro or by producing it in a host cell by genetic engineering.

With respect to the genetic engineering, specifically, after TROP2 cDNA is integrated into a vector capable of expressing TROP2 cDNA, the antigen can be obtained by synthesizing it in a solution containing an enzyme, a substrate and an energy substance required for transcription and translation, or by expressing TROP2 in another prokaryotic or eukaryotic transformed host cell.

Further, the antigen can also be obtained as a secretory protein by expressing a fusion protein obtained by ligating the extracellular domain of TROP2, which is a membrane protein, to the constant region of an antibody in an appropriate host-vector system.

TROP2 cDNA can be obtained by, for example, a so-called PCR method in which a polymerase chain reaction (hereinafter referred to as “PCR”; see Saiki, R. K., et al., Science, (1988) 239, pp. 487-489) is performed using a cDNA library expressing TROP2 cDNA as a template and primers which specifically amplify TROP2 cDNA.

As the in vitro synthesis of the polypeptide, for example, Rapid Translation System (RTS) manufactured by Roche Diagnostics, Inc. can be exemplified, but it is not limited thereto.

Examples of the prokaryotic host cells include Escherichia coli and Bacillus subtilis. In order to transform the host cells with a target gene, the host cells are transformed by a plasmid vector comprising a replicon, i.e., a replication origin derived from a species compatible with the host, and a regulatory sequence. Further, the vector preferably has a sequence capable of imposing phenotypic selectivity on the transformed cell.

Examples of the eukaryotic host cells include vertebrate cells, insect cells, and yeast cells. As the vertebrate cells, for example, simian COS cells (Gluzman, Y., Cell, (1981) 23, pp. 175-182, ATCC CRL-1650; ATCC: American Type Culture Collection), murine fibroblasts NIH3T3 (ATCC No. CRL-1658), and dihydrofolate reductase-deficient strains (Urlaub, G. and Chasin, L. A., Proc. Natl. Acad. Sci. USA (1980) 77, pp. 4126-4220) of Chinese hamster ovarian cells (CHO cells; ATCC: CCL-61); and the like are often used, however, the cells are not limited thereto.

The thus obtained transformant can be cultured according to a method usually carried out in the art, and by the culturing of the transformant, a target polypeptide is produced intracellularly or extracellularly.

A suitable medium to be used for the culturing can be selected by those skilled in the art from various commonly used culture media depending on the employed host cells. If Escherichia coli is employed, for example, an LB medium supplemented with an antibiotic such as ampicillin or IPMG as needed can be used.

A recombinant protein produced intracellularly or extracellularly by the transformant through such culturing can be separated and purified by any of various known separation methods utilizing the physical or chemical property of the protein.

Specific examples of the methods include treatment with a common protein precipitant, ultrafiltration, various types of liquid chromatography such as molecular sieve chromatography (gel filtration), adsorption chromatography, ion exchange chromatography, and affinity chromatography, dialysis, and a combination thereof.

Further, by attaching a tag of six histidine residues to a recombinant protein to be expressed, the protein can be efficiently purified with a nickel affinity column. Alternatively, by attaching the IgG Fc region to a recombinant protein to be expressed, the protein can be efficiently purified with a protein A column.

By combining the above-described methods, a large amount of a target polypeptide can be easily produced in high yield and high purity.

The above-described transformant itself can be also used as the antigen. Alternatively, a cell line expressing TROP2 may be used as the antigen. Examples of such a cell line can include human lung cancer lines NCI-H322, PC14, NCIH-H2122, and LCAM1, a human prostate cancer line PC3, human pancreatic cancer lines BxPC-3, Capan-1, and PK-1, a human ovarian cancer line SKOV3, and a human colorectal cancer line COLO205, though the cell line according to the present invention is not limited to these cell lines as long as expressing TROP2.

### Production of Anti-TROP2 Monoclonal Antibody

Examples of the antibody specifically bind to TROP2 include a monoclonal antibody specifically bind to TROP2, and a method of obtaining such antibody is as described below.

The production of a monoclonal antibody generally requires the following operational steps of:
(a) purifying a biopolymer to be used as an antigen, or preparing antigen-expressing cells;
(b) preparing antibody-producing cells by immunizing an animal by injection of the antigen, collecting the blood, assaying its antibody titer to determine when the spleen is excised;
(c) preparing myeloma cells (hereinafter referred to as “myeloma”);
(d) fusing the antibody-producing cells with the myeloma;
(e) screening a group of hybridomas producing a desired antibody;
(f) dividing the hybridomas into single cell clones (cloning);
(g) optionally, culturing the hybridoma or rearing an animal implanted with the hybridoma for producing a large amount of monoclonal antibody;
(h) examining the thus produced monoclonal antibody for biological activity and binding specificity, or assaying the same for properties as a labeled reagent; and the like.

Hereinafter, the method of producing a monoclonal antibody will be described in detail following the above steps, however, the method is not limited thereto, and, for example, antibody-producing cells other than spleen cells and myeloma can be used.

### (A) Purification of Antigen

As the antigen, TROP2 prepared by the method as described above or a partial peptide thereof can be used.

Further, a membrane fraction prepared from recombinant cells expressing TROP2 or the recombinant cells expressing TROP2 themselves, and also a partial peptide of the protein of the invention chemically synthesized by a method known to those skilled in the art can also be used as the antigen.

Further, a cell line expressing TROP2 can be also used as the antigen.

### (B) Preparation of Antibody-Producing Cells

The antigen obtained in the step (a) is mixed with an adjuvant such as Freund’s complete or incomplete adjuvant or auxiliary agent such as aluminum potassium sulfate and the resulting mixture is used as an immunogen to immunize an experimental animal. In an alternative method, the experimental animal is immunized with antigen-expressing cells as an immunogen. As the experimental animal, any animal used in a known hybridoma production method can be used without hindrance. Specifically, for example, a mouse, a rat, a goat, sheep, cattle, a horse, or the like can be used. However, from the viewpoint of ease of availability of myeloma cells to be fused with the extracted antibody-producing cells, a mouse or a rat is preferably used as the animal to be immunized.

Further, the strain of a mouse or a rat to be used is not particularly limited, and in the case of a mouse, for example, various strains such as A, AKR, BALB/c, BDP, BA, CE, C3H, 57BL, C57BL, C57L, DBA, FL, HTH, HT1, LP, NZB, NZW, RF, R III, SJL, SWR, WB, and 129 and the like can be used, and in the case of a rat, for example, Wistar, Low, Lewis, Sprague, Dawley, ACI, BN, Fischer and the like can be used.

These mice and rats can be obtained from breeders/distributors of experimental animals, for example, CLEA Japan, Inc. and Charles River Laboratories Japan, Inc.

In consideration of compatibility of fusing with myeloma cells described below, in the case of a mouse, BALB/c strain, and in the case of a rat, Wistar and Low strains are particularly preferred as the animal to be immunized.

Further, in consideration of antigenic homology between humans and mice, it is also preferred to use a mouse having decreased biological function to remove auto-antibodies, that is, a mouse with an autoimmune disease.

The age of such mouse or rat at the time of immunization is preferably 5 to 12 weeks of age, more preferably 6 to 8 weeks of age.

In order to immunize an animal with TROP2 or a recombinant thereof, for example, a known method described in detail in, for example, Weir, D. M., Handbook of Experimental Immunology Vol. I. II. III., Blackwell Scientific Publications, Oxford (1987); Kabat, E. A. and Mayer, M. M., Experimental Immunochemistry, Charles C Thomas Publisher Springfield, Illinois (1964) or the like can be used.

Among these immunization methods, a preferred specific method in the present invention is, for example, as follows.

That is, first, a membrane protein fraction serving as the antigen or cells caused to express the antigen is/are intradermally or intraperitoneally administrated to an animal. However, the combination of both routes of administration is preferred for increasing the immunization efficiency, and when intradermal administration is performed in the first half and intraperitoneal administration is performed in the latter half or only at the last dosing, the immunization efficiency can be particularly increased.

The administration schedule of the antigen varies depending on the type of animal to be immunized, individual difference or the like. However, in general, an administration schedule in which the frequency of administration of the antigen is 3 to 6 times and the dosing interval is 2 to 6 weeks is preferred, and an administration schedule in which the frequency of administration of the antigen is 3 to 4 times and the dosing interval is 2 to 4 weeks is more preferred.

Further, the dose of the antigen varies depending on the type of animal, individual differences or the like, however, the dose is generally set to 0.05 to 5 mg, preferably about 0.1 to 0.5 mg.

A booster immunization is performed 1 to 6 weeks, preferably 1 to 4 weeks, more preferably 1 to 3 weeks after the administration of the antigen as described above. When the immunogen is cells, 1 × 10⁶ to 1 × 10⁷ cells are employed.

The dose of the antigen at the time of performing the booster immunization varies depending on the type or size of animal or the like, however, in the case of, for example, a mouse, the dose is generally set to 0.05 to 5 mg, preferably 0.1 to 0.5 mg, more preferably about 0.1 to 0.2 mg. When the immunogen is cells, 1 × 10⁶ to 1 × 10⁷ cells are employed.

Spleen cells or lymphocytes including antibody-producing cells are aseptically removed from the immunized animal after 1 to 10 days, preferably 2 to 5 days, more preferably 2 to 3 days from the booster immunization. At this time, the antibody titer is measured, and if an animal having a sufficiently increased antibody titer is used as a supply source of the antibody-producing cells, the subsequent procedure can be carried out more efficiently.

Examples of the method of measuring the antibody titer to be used here include an RIA method and an ELISA method, but the method is not limited thereto. For example, if an ELISA method is employed, the measurement of the antibody titer in the invention can be carried out according to the procedures as described below.

First, a purified or partially purified antigen is adsorbed to the surface of a solid phase such as a 96-well plate for ELISA, and the surface of the solid phase having no antigen adsorbed thereto is covered with a protein unrelated to the antigen such as bovine serum albumin (BSA). After washing the surface, the surface is brought into contact with a serially-diluted sample (for example, mouse serum) as a primary antibody to allow the antibody in the sample to bind to the antigen.

Further, as a secondary antibody, an antibody labeled with an enzyme against a mouse antibody is added and is allowed to bind to the mouse antibody. After washing, a substrate for the enzyme is added and a change in absorbance which occurs due to color development induced by degradation of the substrate or the like is measured and the antibody titer is calculated based on the measurement.

The separation of the antibody-producing cells from the spleen cells or lymphocytes of the immunized animal can be carried out according to a known method (for example, Kohler et al., Nature (1975), 256, p. 495; Kohler et al., Eur. J. Immunol. (1977), 6, p. 511; Milstein et al., Nature (1977), 266, p. 550; Walsh, Nature (1977), 266, p. 495). For example, in the case of spleen cells, a general method in which the antibody-producing cells are separated by homogenizing the spleen to obtain the cells through filtration with a stainless steel mesh and suspending the cells in Eagle’s Minimum Essential Medium (MEM) can be employed.

### (C) Preparation of Myeloma Cells (Hereinafter Referred to as “Myeloma”)

The myeloma cells to be used for cell fusion are not particularly limited and suitable cells can be selected from known cell lines. However, in consideration of convenience when a hybridoma is selected from fused cells, it is preferred to use an HGPRT (hypoxanthine-guanine phosphoribosyl transferase) deficient strain whose selection procedure has been established.

More specifically, examples of the HGPRT-deficient strain include X63-Ag8(X63), NS1-ANS/1(NS1), P3X63-Ag8.U1(P3U1), X63-Ag8.653(X63.653), SP2/0-Ag14(SP2/0), MPC11-45.6TG1.7(45.6TG), FO, S149/5XXO, and BU.1 derived from mice; 210.RSY3.Ag.1.2.3(Y3) derived from rats; and U266AR(SKO-007), GM1500·GTG-A12(GM1500), UC729-6, LICR-LOW-HMy2(HMy2) and 8226AR/NIP4-1(NP41) derived from humans. These HGPRT-deficient strains are available from, for example, ATCC or the like.

These cell strains are subcultured in an appropriate medium such as an 8-azaguanine medium (a medium obtained by adding 8-azaguanine to an RPMI 1640 medium supplemented with glutamine, 2-mercaptoethanol, gentamicin, and fetal calf serum (hereinafter referred to as “FBS”), Iscove’s Modified Dulbecco’s Medium (IMDM), or Dulbecco’s Modified Eagle Medium (DMEM). In this case, 3 to 4 days before performing cell fusion, the cells are subcultured in a normal medium (for example, an ASF104 medium (manufactured by Ajinomoto Co., Ltd.) containing 10% FCS) to ensure not less than 2 x 10⁷ cells on the day of cell fusion.

### (D) Cell Fusion

Fusion between the antibody-producing cells and the myeloma cells can be appropriately performed according to a known method (Weir, D. M. Handbook of Experimental Immunology Vol. I. II. III., Blackwell Scientific Publications, Oxford (1987); Kabat, E. A. and Mayer, M. M., Experimental Immunochemistry, Charles C Thomas Publisher, Springfield, Illinois (1964), etc.), under conditions such that the survival rate of cells is not excessively reduced.

As such a method, for example, a chemical method in which the antibody-producing cells and the myeloma cells are mixed in a solution containing a polymer such as polyethylene glycol at a high concentration, a physical method using electric stimulation, or the like can be used. Among these methods, a specific example of the chemical method is as described below.

That is, in the case where polyethylene glycol is used in the solution containing a polymer at a high concentration, the antibody-producing cells and the myeloma cells are mixed in a solution of polyethylene glycol having a molecular weight of 1500 to 6000, more preferably 2000 to 4000 at a temperature of from 30 to 40° C., preferably from 35 to 38° C. for 1 to 10 minutes, preferably 5 to 8 minutes.

### (E) Selection of a Group of Hybridomas

The method of selecting hybridomas obtained by the above-described cell fusion is not particularly limited. Usually, an HAT (hypoxanthine, aminopterin, thymidine) selection method (Kohler et al., Nature (1975), 256, p. 495; Milstein et al., Nature (1977), 266, p. 550) is used.

This method is effective when hybridomas are obtained using the myeloma cells of an HGPRT-deficient strain which cannot survive in the presence of aminopterin. That is, by culturing unfused cells and hybridomas in an HAT medium, only hybridomas resistant to aminopterin are selectively allowed to survive and proliferate.

### (F) Division Into Single Cell Clone (Cloning)

As a cloning method for hybridomas, a known method such as a methylcellulose method, a soft agarose method, or a limiting dilution method can be used (see, e.g., Barbara, B. M. and Stanley, M. S.: Selected Methods in Cellular Immunology, W. H. Freeman and Company, San Francisco (1980)). Among these methods, particularly, a three-dimensional culture method such as a methylcellulose method is preferred. For example, the group of hybridomas produced by cell fusion are suspended in a methylcellulose medium such as ClonaCell-HY Selection Medium D (manufactured by StemCell Technologies, Inc., #03804) and cultured. Then, the formed hybridoma colonies are collected, whereby monoclonal hybridomas can be obtained. The collected respective hybridoma colonies are cultured, and a hybridoma which has been confirmed to have a stable antibody titer in an obtained hybridoma culture supernatant is selected as a TROP2 monoclonal antibody-producing hybridoma strain.

Examples of the thus established hybridoma strain include TROP2 hybridoma TINA1. In this specification, an antibody produced by the TROP2 hybridoma TINA1 is referred to as “TINA1 antibody” or simply “TINA1”.

The heavy chain variable region of the TINA1 antibody has an amino acid sequence represented by SEQ ID NO: 2 in the Sequence Listing. Further, the light chain variable region of the TINA1 antibody has an amino acid sequence represented by SEQ ID NO: 4 in the Sequence Listing.

### (G) Preparation of Monoclonal Antibody by Culturing Hybridoma

By culturing the thus selected hybridoma, a monoclonal antibody can be efficiently obtained. However, prior to culturing, it is preferred to perform screening of a hybridoma which produces a target monoclonal antibody.

In such screening, a known method can be employed.

The measurement of the antibody titer in the invention can be carried out by, for example, an ELISA method explained in item (b) described above.

The hybridoma obtained by the method described above can be stored in a frozen state in liquid nitrogen or in a freezer at -80° C. or below.

After completion of cloning, the medium is changed from an HT medium to a normal medium, and the hybridoma is cultured.

Large-scale culture is performed by rotation culture using a large culture bottle or by spinner culture. From the supernatant obtained by the large-scale culture, a monoclonal antibody which specifically binds to the protein of the invention can be obtained by purification using a method known to those skilled in the art such as gel filtration.

Further, the hybridoma is injected into the abdominal cavity of a mouse of the same strain as the hybridoma (for example, the above-described BALB/c) or a Nu/Nu mouse to proliferate the hybridoma, whereby the ascites containing a large amount of the monoclonal antibody of the invention can be obtained.

In the case where the hybridoma is administrated in the abdominal cavity, if a mineral oil such as 2,6,10,14-tetramethyl pentadecane (pristane) is administrated 3 to 7 days prior thereto, a larger amount of the ascites can be obtained.

For example, an immunosuppressant is previously injected into the abdominal cavity of a mouse of the same strain as the hybridoma to inactivate T cells. 20 days thereafter, 10⁶ to 10⁷ hybridoma clone cells are suspended in a serum-free medium (0.5 ml), and the suspension is administrated in the abdominal cavity of the mouse. In general, when the abdomen is expanded and filled with the ascites, the ascites is collected from the mouse. By this method, the monoclonal antibody can be obtained at a concentration which is about 100 times or much higher than that in the culture solution.

The monoclonal antibody obtained by the above-described method can be purified by a method described in, for example, Weir, D. M.: Handbook of Experimental Immunology Vol. I, II, III, Blackwell Scientific Publications, Oxford (1978).

The thus obtained monoclonal antibody has high antigen specificity for TROP2.

### (H) Assay of Monoclonal Antibody

The isotype and subclass of the thus obtained monoclonal antibody can be determined as follows.

First, examples of the identification method include an Ouchterlony method, an ELISA method, and an RIA method.

An Ouchterlony method is simple, but when the concentration of the monoclonal antibody is low, a condensation operation is required.

On the other hand, when an ELISA method or an RIA method is used, by directly reacting the culture supernatant with an antigen-adsorbed solid phase and using antibodies corresponding to various types of immunoglobulin isotypes and subclasses as secondary antibodies, the isotype and subclass of the monoclonal antibody can be identified.

In addition, as a simpler method, a commercially available identification kit (for example, Mouse Typer Kit manufactured by Bio-Rad Laboratories, Inc.) or the like can also be used.

Further, the quantitative determination of a protein can be performed by the Folin Lowry method and a method of calculation based on the absorbance at 280 nm (1.4 (OD 280) = Immunoglobulin 1 mg/ml).

Further, even when the monoclonal antibody is separately and independently obtained by performing again the steps of (a) to (h) in (2), it is possible to obtain an antibody having a cytotoxic activity equivalent to that of the TINA1 antibody or an antibody comprising a heavy chain comprising SEQ ID NO: 45 and a light chain comprising SEQ ID NO: 46. As one example of such an antibody, an antibody which binds to the same epitope as the TINA1 antibody or an antibody comprising a heavy chain comprising SEQ ID NO: 45 and a light chain comprising SEQ ID NO: 46. If a newly produced monoclonal antibody binds to a partial peptide or a partial tertiary structure to which the TINA1 antibody or an antibody comprising a heavy chain comprising SEQ ID NO: 45 and a light chain comprising SEQ ID NO: 46, it can be determined that the monoclonal antibody binds to the same epitope. Further, by confirming that the monoclonal antibody competes with the TINA1 antibody or an antibody comprising a heavy chain comprising SEQ ID NO: 45 and a light chain comprising SEQ ID NO: 46 for the binding to TROP2 (that is, the monoclonal antibody inhibits the binding between the TINA1 antibody or an antibody comprising a heavy chain comprising SEQ ID NO: 45 and a light chain comprising SEQ ID NO: 46 and TROP2), it can be determined that the monoclonal antibody binds to the same epitope as the anti-TROP2 antibody even if the specific epitope sequence or structure has not been determined. When it is confirmed that the monoclonal antibody binds to the same epitope as the anti-TROP2 antibody, the monoclonal antibody is strongly expected to have the antigen-binding affinity and a biological activity equivalent to that of the TINA1 antibody or an antibody comprising a heavy chain comprising SEQ ID NO: 45 and a light chain comprising SEQ ID NO: 46.

### Other Antibodies

The antibody of the invention includes not only the above-described monoclonal antibody against TROP2 but also a recombinant antibody obtained by artificial modification for the purpose of decreasing heterologous antigenicity to humans such as a chimeric antibody, a humanized antibody and a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (see Proc. Natl. Acad. Sci. USA, 81, 6851-6855, (1984)).

As the humanized antibody, an antibody obtained by integrating only a complementarity determining region (CDR) into a human-derived antibody (see Nature (1986) 321, pp. 522-525), and an antibody obtained by grafting a part of the amino acid residues of the framework as well as the CDR sequence to a human antibody by a CDR-grafting method (International Publication No. WO 90/07861) can be exemplified.

However, the humanized antibody derived from the TINA1 antibody is not limited to a specific humanized antibody as long as the humanized antibody has all 6 types of CDR sequences of the TINA1 antibody. The heavy chain variable region of the TINA1 antibody has CDRH1 (TAGMQ) consisting of an amino acid sequence represented by SEQ ID NO: 23 in the Sequence Listing, CDRH2 (WINTHSGVPKYAEDFKG) consisting of an amino acid sequence represented by SEQ ID NO: 24 in the Sequence Listing, and CDRH3 (SGFGSSYWYFDV) consisting of an amino acid sequence represented by SEQ ID NO: 25 in the Sequence Listing. Further, the light chain variable region of the TINA1 antibody has CDRL1 (KASQDVSTAVA) consisting of an amino acid sequence represented by SEQ ID NO: 26 in the Sequence Listing, CDRL2 (SASYRYT) consisting of an amino acid sequence represented by SEQ ID NO: 27 in the Sequence Listing, and CDRL3 (QQHYITPLT) consisting of an amino acid sequence represented by SEQ ID NO: 28 in the Sequence Listing.

As an example of the humanized antibody of a mouse antibody TINA1, an arbitrary combination of a heavy chain comprising a heavy chain variable region consisting of any one of (1) an amino acid sequence consisting of amino acid residues 20 to 140 of SEQ ID NO: 12, 14, or 16 or amino acid residues 1-121 of SEQ ID NO: 45 in the Sequence Listing, (2) an amino acid sequence having a homology of at least 95% or more with the amino acid sequence (1) described above, and (3) an amino acid sequence wherein one or several amino acids in the amino acid sequence (1) described above are deleted, substituted or added and a light chain comprising a light chain variable region consisting of any one of (4) an amino acid sequence consisting of amino acid residues 21 to 129 of SEQ ID NO: 18, 20,or 22 or amino acid residues 1-109 of SEQ ID NO: 46 in the Sequence Listing, (5) an amino acid sequence having a homology of at least 95% or more with the amino acid sequence (4) described above, and (6) an amino acid sequence wherein one or several amino acids in the amino acid sequence (4) described above are deleted, substituted or added can be exemplified.

As an antibody which has a preferred combination of a heavy chain and a light chain described above, an antibody comprising a heavy chain comprising a variable region comprising amino acids 1-121 of SEQ ID NO: 45 and a light chain comprising a variable region comprising amino acids 1-109 of SEQ ID NO: 46; an antibody consisting of a heavy chain comprising a variable region consisting of an amino acid sequence consisting of amino acid positions 20 to 140 of SEQ ID NO: 12 and a light chain comprising a variable region consisting of an amino acid sequence consisting of amino acid positions 21 to 129 of SEQ ID NO: 18; an antibody consisting of a heavy chain comprising a variable region consisting of an amino acid sequence consisting of amino acid positions 20 to 140 of SEQ ID NO: 12 and a light chain comprising a variable region consisting of an amino acid sequence consisting of amino acid positions 21 to 129 of SEQ ID NO: 20; an antibody consisting of a heavy chain comprising a variable region consisting of an amino acid sequence consisting of amino acid positions 20 to 140 of SEQ ID NO: 12 and a light chain comprising a variable region consisting of an amino acid sequence consisting of amino acid positions 21 to 129 of SEQ ID NO: 22; an antibody consisting of a heavy chain comprising a variable region consisting of an amino acid sequence consisting of amino acid positions 20 to 140 of SEQ ID NO: 14 and a light chain comprising a variable region consisting of an amino acid sequence consisting of amino acid positions 21 to 129 of SEQ ID NO: 18; an antibody consisting of a heavy chain comprising a variable region consisting of an amino acid sequence consisting of amino acid positions 20 to 140 of SEQ ID NO: 14 and a light chain comprising a variable region consisting of an amino acid sequence consisting of amino acid positions 21 to 129 of SEQ ID NO: 20; an antibody consisting of a heavy chain comprising a variable region consisting of an amino acid sequence consisting of amino acid positions 20 to 140 of SEQ ID NO: 14 and a light chain comprising a variable region consisting of an amino acid sequence consisting of amino acid positions 21 to 129 of SEQ ID NO: 22; an antibody consisting of a heavy chain comprising a variable region consisting of an amino acid sequence consisting of amino acid positions 20 to 140 of SEQ ID NO: 16 and a light chain comprising a variable region consisting of an amino acid sequence consisting of amino acid positions 21 to 129 of SEQ ID NO: 18; an antibody consisting of a heavy chain comprising a variable region consisting of an amino acid sequence consisting of amino acid positions 20 to 140 of SEQ ID NO: 16 and a light chain comprising a variable region consisting of an amino acid sequence consisting of amino acid positions 21 to 129 of SEQ ID NO: 20; and an antibody consisting of a heavy chain comprising a variable region consisting of an amino acid sequence consisting of amino acid positions 20 to 140 of SEQ ID NO: 16 and a light chain comprising a variable region consisting of an amino acid sequence consisting of amino acid positions 21 to 129 of SEQ ID NO: 22 can be exemplified.

Further, as an antibody which has a more preferred combination of a heavy chain and a light chain described above, an antibody comprising a heavy chain comprising SEQ ID NO: 45 and a light chain comprising SEQ ID NO: 46; an antibody consisting of a heavy chain consisting of an amino acid sequence consisting of amino acid positions 20 to 470 of SEQ ID NO: 12 and a light chain consisting of an amino acid sequence consisting of amino acid positions 21 to 234 of SEQ ID NO: 18; an antibody consisting of a heavy chain consisting of an amino acid sequence consisting of amino acid positions 20 to 470 of SEQ ID NO: 12 and a light chain consisting of an amino acid sequence consisting of amino acid positions 21 to 234 of SEQ ID NO: 20; an antibody consisting of a heavy chain consisting of an amino acid sequence consisting of amino acid positions 20 to 470 of SEQ ID NO: 12 and a light chain consisting of an amino acid sequence consisting of amino acid positions 21 to 234 of SEQ ID NO: 22; an antibody consisting of a heavy chain consisting of an amino acid sequence consisting of amino acid positions 20 to 470 of SEQ ID NO: 14 and a light chain consisting of an amino acid sequence consisting of amino acid positions 21 to 234 of SEQ ID NO: 18; an antibody consisting of a heavy chain consisting of an amino acid sequence consisting of amino acid positions 20 to 470 of SEQ ID NO: 14 and a light chain consisting of an amino acid sequence consisting of amino acid positions 21 to 234 of SEQ ID NO: 20; an antibody consisting of a heavy chain consisting of an amino acid sequence consisting of amino acid positions 20 to 470 of SEQ ID NO: 14 and a light chain consisting of an amino acid sequence consisting of amino acid positions 21 to 234 of SEQ ID NO: 22; an antibody consisting of a heavy chain consisting of an amino acid sequence consisting of amino acid positions 20 to 470 of SEQ ID NO: 16 and a light chain consisting of an amino acid sequence consisting of amino acid positions 21 to 234 of SEQ ID NO: 18; an antibody consisting of a heavy chain consisting of an amino acid sequence consisting of amino acid positions 20 to 470 of SEQ ID NO: 16 and a light chain consisting of an amino acid sequence consisting of amino acid positions 21 to 234 of SEQ ID NO: 20; and an antibody consisting of a heavy chain consisting of an amino acid sequence consisting of amino acid positions 20 to 470 of SEQ ID NO: 16 and a light chain consisting of an amino acid sequence consisting of amino acid positions 21 to 234 of SEQ ID NO: 22 can be exemplified.

As an antibody which has a superior preferred combination of a heavy chain and a light chain described above, an antibody consisting of a heavy chain comprising a variable region comprising amino acids 1-121 of SEQ ID NO: 45 and a light chain comprising a variable region comprising amino acids 1-109 of SEQ ID NO: 46; an antibody consisting of a heavy chain comprising a variable region consisting of an amino acid sequence consisting of amino acid residues 20 to 140 of SEQ ID NO: 12 and a light chain comprising a variable region consisting of an amino acid sequence consisting of amino acid residues 21 to 129 of SEQ ID NO: 18; an antibody consisting of a heavy chain comprising a variable region consisting of an amino acid sequence consisting of amino acid residues 20 to 140 of SEQ ID NO: 14 and a light chain comprising a variable region consisting of an amino acid sequence consisting of amino acid residues 21 to 129 of SEQ ID NO: 18; an antibody consisting of a heavy chain comprising a variable region consisting of an amino acid sequence consisting of amino acid residues 20 to 140 of SEQ ID NO: 14 and a light chain comprising a variable region consisting of an amino acid sequence consisting of amino acid residues 21 to 129 of SEQ ID NO: 20; and an antibody consisting of a heavy chain comprising a variable region consisting of an amino acid sequence consisting of amino acid residues 20 to 140 of SEQ ID NO: 16 and a light chain comprising a variable region consisting of an amino acid sequence consisting of amino acid residues 21 to 129 of SEQ ID NO: 22 can be exemplified.

Furthermore, as an antibody which has another more preferred combination of a heavy chain and a light chain described above, an antibody consisting of a heavy chain comprising SEQ ID NO: 45 and a light chain comprising SEQ ID NO: 46; an antibody consisting of a heavy chain consisting of an amino acid sequence of SEQ ID NO: 12 and a light chain consisting of an amino acid sequence of SEQ ID NO: 18; an antibody consisting of a heavy chain consisting of an amino acid sequence of SEQ ID NO: 12 and a light chain consisting of an amino acid sequence of SEQ ID NO: 20; an antibody consisting of a heavy chain consisting of an amino acid sequence of SEQ ID NO: 12 and a light chain consisting of an amino acid sequence of SEQ ID NO: 22; an antibody consisting of a heavy chain consisting of an amino acid sequence of SEQ ID NO: 14 and a light chain consisting of an amino acid sequence of SEQ ID NO: 18; an antibody consisting of a heavy chain consisting of an amino acid sequence of SEQ ID NO: 14 and a light chain consisting of an amino acid sequence of SEQ ID NO: 20; an antibody consisting of a heavy chain consisting of an amino acid sequence of SEQ ID NO: 14 and a light chain consisting of an amino acid sequence of SEQ ID NO: 22; an antibody consisting of a heavy chain consisting of an amino acid sequence of SEQ ID NO: 16 and a light chain consisting of an amino acid sequence of SEQ ID NO: 18; an antibody consisting of a heavy chain consisting of an amino acid sequence of SEQ ID NO: 16 and a light chain consisting of an amino acid sequence of SEQ ID NO: 20; and an antibody consisting of a heavy chain consisting of an amino acid sequence of SEQ ID NO: 16 and a light chain consisting of an amino acid sequence of SEQ ID NO: 22 can be exemplified.

As an antibody which has a superior preferred combination of a heavy chain and a light chain described above, an antibody comprising a heavy chain comprising SEQ ID NO: 45 and a light chain comprising SEQ ID NO: 46; an antibody consisting of a heavy chain consisting of an amino acid sequence consisting of amino acid positions 20 to 470 of SEQ ID NO: 12 and a light chain consisting of an amino acid sequence consisting of amino acid positions 21 to 234 of SEQ ID NO: 18; an antibody consisting of a heavy chain consisting of an amino acid sequence consisting of amino acid positions 20 to 470 of SEQ ID NO: 14 and a light chain consisting of an amino acid sequence consisting of amino acid positions 21 to 234 of SEQ ID NO: 18; an antibody consisting of a heavy chain consisting of an amino acid sequence consisting of amino acid positions 20 to 470 of SEQ ID NO: 14 and a light chain consisting of an amino acid sequence consisting of amino acid positions 21 to 234 of SEQ ID NO: 20; and an antibody consisting of a heavy chain consisting of an amino acid sequence consisting of amino acid positions 20 to 470 of SEQ ID NO: 16 and a light chain consisting of an amino acid sequence consisting of amino acid positions 21 to 234 of SEQ ID NO: 22 can be exemplified.

Further, as an antibody which has a more superior preferred combination of a heavy chain and a light chain described above, an antibody consisting of a heavy chain consisting of an amino acid sequence consisting of amino acid positions 20 to 469 of SEQ ID NO: 12 and a light chain consisting of an amino acid sequence consisting of amino acid positions 21 to 234 of SEQ ID NO: 18; an antibody consisting of a heavy chain consisting of an amino acid sequence consisting of amino acid positions 20 to 469 of SEQ ID NO: 14 and a light chain consisting of an amino acid sequence consisting of amino acid positions 21 to 234 of SEQ ID NO: 18; an antibody consisting of a heavy chain consisting of an amino acid sequence consisting of amino acid positions 20 to 469 of SEQ ID NO: 14 and a light chain consisting of an amino acid sequence consisting of amino acid positions 21 to 234 of SEQ ID NO: 20; and an antibody consisting of a heavy chain consisting of an amino acid sequence consisting of amino acid positions 20 to 469 of SEQ ID NO: 16 and a light chain consisting of an amino acid sequence consisting of amino acid positions 21 to 234 of SEQ ID NO: 22 can be exemplified.

By combining a sequence having a high homology with the above-described heavy chain amino acid sequence with a sequence having a high homology with the above-described light chain amino acid sequence, it is possible to select an antibody having a biological activity equivalent to that of each of the above-described antibodies. Such a homology is generally a homology of 80% or more, preferably a homology of 90% or more, more preferably a homology of 95% or more, most preferably a homology of 99% or more. Further, by combining an amino acid sequence wherein one to several amino acid residues are substituted, deleted or added in the heavy chain or light chain amino acid sequence, it is also possible to select an antibody having a biological activity equivalent to that of each of the above-described antibodies.

The homology between two amino acid sequences can be determined using default parameters of Blast algorithm version 2.2.2 (Altschul, Stephen F., Thomas L. Madden, Alejandro A. Schaeffer, Jinghui Zhang, Zheng Zhang, Webb Miller, and David J. Lipman (1997), “Gapped BLAST and PSI-BLAST: a new generation of protein database search programs”, Nucleic Acids Res. 25: 3389-3402). The Blast algorithm can be used also through the Internet by accessing the site ncbi.nlm.nih.gov/blast.

In the heavy chain amino acid sequence represented by SEQ ID NO: 12, 14, or 16 in the Sequence Listing, an amino acid sequence consisting of amino acid residues 1 to 19 is a signal sequence, an amino acid sequence consisting of amino acid residues 20 to 140 is a variable region, and an amino acid sequence consisting of amino acid residues 141 to 470 is a constant region.

Further, in the light chain amino acid sequence represented by SEQ ID NO: 18, 20 or 22 in the Sequence Listing, an amino acid sequence consisting of amino acid residues 1 to 20 is a signal sequence, an amino acid sequence consisting of amino acid residues 21 to 129 is a variable region, and an amino acid sequence consisting of amino acid residues 130 to 234 is a constant region.

Further, the antibody of the invention includes a human antibody which binds to TROP2. An anti-TROP2 human antibody refers to a human antibody having only a sequence of an antibody derived from a human chromosome. The anti-TROP2 human antibody can be obtained by a method using a human antibody-producing mouse having a human chromosome fragment comprising heavy and light chain genes of a human antibody (see Tomizuka, K. et al., Nature Genetics (1997) 16, pp. 133-143; Kuroiwa, Y. et al., Nucl. Acids Res. (1998) 26, pp. 3447-3448; Yoshida, H. et al., Animal Cell Technology: Basic and Applied Aspects vol. 10, pp. 69-73 (Kitagawa, Y., Matuda, T. and Iijima, S. eds.), Kluwer Academic Publishers, 1999; Tomizuka, K. et al., Proc. Natl. Acad. Sci. USA (2000) 97, pp. 722-727, etc.).

Such a human antibody-producing mouse can be created specifically as follows. A genetically modified animal in which endogenous immunoglobulin heavy and light chain gene loci have been disrupted, and instead, human immunoglobulin heavy and light chain gene loci have been introduced via a yeast artificial chromosome (YAC) vector or the like is created by producing a knockout animal and a transgenic animal and mating these animals.

Further, according to a recombinant DNA technique, by using cDNAs encoding each of such a heavy chain and a light chain of a human antibody, and preferably a vector comprising such cDNAs, eukaryotic cells are transformed, and a transformant cell which produces a recombinant human monoclonal antibody is cultured, whereby the antibody can also be obtained from the culture supernatant.

Here, as the host, for example, eukaryotic cells, preferably mammalian cells such as CHO cells, lymphocytes, or myeloma cells can be used.

Further, a method of obtaining a phage display-derived human antibody selected from a human antibody library (see Wormstone, I. M. et al., Investigative Ophthalmology & Visual Science. (2002) 43 (7), pp. 2301-2308; Carmen, S. et al., Briefings in Functional Genomics and Proteomics (2002), 1 (2), pp. 189-203; Siriwardena, D. et al., Ophthalmology (2002) 109 (3), pp. 427-431, etc.) is also known.

For example, a phage display method in which a variable region of a human antibody is expressed on the surface of a phage as a single-chain antibody (scFv), and a phage which binds to an antigen is selected (Nature Biotechnology (2005), 23, (9), pp. 1105-1116) can be used.

By analyzing the gene of the phage selected based on the binding to an antigen, a DNA sequence encoding the variable region of a human antibody which binds to an antigen can be determined.

If the DNA sequence of scFv which binds to an antigen is determined, a human antibody can be obtained by preparing an expression vector comprising the sequence and introducing the vector into an appropriate host to express it (International Publication No. WO 92/01047, WO 92/20791, WO 93/06213, WO 93/11236, WO 93/19172, WO 95/01438, WO 95/15388; Annu. Rev. Immunol. (1994) 12, pp. 433-455; Nature Biotechnology (2005) 23 (9), pp. 1105-1116).

If a newly produced human antibody binds to a partial peptide or a partial tertiary structure to which the TINA1 antibody binds, it can be determined that the human antibody binds to the same epitope as the TINA1 antibody. Further, by confirming that the human antibody competes with the TINA1 antibody for the binding to TROP2 (that is, the human antibody inhibits the binding between the TINA1 antibody and TROP2), it can be determined that the human antibody binds to the same epitope as the TINA1 antibody even if the specific epitope sequence or structure has not been determined. When it is confirmed that the human antibody binds to the same epitope as the TINA1 antibody, the human antibody is strongly expected to have a biological activity equivalent to that of the TINA1 antibody.

The chimeric antibodies, humanized antibodies, or human antibodies obtained by the above-described method can be evaluated for the binding property to an antigen by a known method or the like, and a preferred antibody can be selected.

As one example of another index for use in the comparison of the properties of antibodies, the stability of antibodies can be exemplified. The differential scanning calorimetry (DSC) is a device capable of quickly and accurately measuring a thermal denaturation midpoint temperature (Tm) to be used as a favorable index of the relative conformational stability of proteins. By measuring the Tm values using DSC and comparing the values, a difference in thermal stability can be compared. It is known that the storage stability of antibodies shows some correlation with the thermal stability of antibodies (Lori Burton, et. al., Pharmaceutical Development and Technology (2007) 12, pp. 265-273), and a preferred antibody can be selected by using thermal stability as an index. Examples of other indices for selecting antibodies include the following features: the yield in an appropriate host cell is high; and the aggregability in an aqueous solution is low. For example, an antibody which shows the highest yield does not always show the highest thermal stability, and therefore, it is necessary to select an antibody most suitable for the administration to humans by making comprehensive evaluation based on the above-described indices.

In the present invention, a modified variant of the antibody is also included. The modified variant refers to a variant obtained by subjecting the antibody of the present invention to chemical or biological modification. Examples of the chemically modified variant include variants chemically modified by linking a chemical moiety to an amino acid skeleton, variants chemically modified with an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell.

Further, an antibody labeled so as to enable the detection or isolation of the antibody or an antigen of the invention, for example, an enzyme-labeled antibody, a fluorescence-labeled antibody, and an affinity-labeled antibody are also included in the meaning of the modified variant. Such a modified variant of the antibody of the invention is useful for improving the stability and blood retention of the antibody, reducing the antigenicity thereof, detecting or isolating an antibody or an antigen, and so on.

Further, by regulating the modification of a glycan which is linked to the antibody of the invention (glycosylation, defucosylation, etc.), it is possible to enhance an antibody-dependent cellular cytotoxic activity. As the technique for regulating the modification of a glycan of antibodies, International Publication No. WO 1999/54342, WO 2000/61739, WO 2002/31140, etc. are known. However, the technique is not limited thereto. In the antibody of the present invention, an antibody in which the modification of a glycan is regulated is also included.

In the case where an antibody is produced by first isolating an antibody gene and then introducing the gene into an appropriate host, a combination of an appropriate host and an appropriate expression vector can be used. Specific examples of the antibody gene include a combination of a gene encoding a heavy chain sequence of an antibody described in this specification and a gene encoding a light chain sequence thereof. When a host cell is transformed, it is possible to insert the heavy chain sequence gene and the light chain sequence gene into the same expression vector, and also into different expression vectors separately.

In the case where eukaryotic cells are used as the host, animal cells, plant cells, and eukaryotic microorganisms can be used. As the animal cells, mammalian cells, for example, simian COS cells (Gluzman, Y., Cell, (1981) 23, pp. 175-182, ATCC CRL-1650), murine fibroblasts NIH3T3 (ATCC No. CRL-1658), and dihydrofolate reductase-deficient strains (Urlaub, G. and Chasin, L. A., Proc. Natl. Acad. Sci. USA (1980) 77, pp. 4126-4220) of Chinese hamster ovarian cells (CHO cells; ATCC: CCL-61) can be exemplified.

In the case where prokaryotic cells are used, for example, Escherichia coli and Bacillus subtilis can be exemplified.

By introducing a desired antibody gene into these cells through transformation, and culturing the thus transformed cells in vitro, the antibody can be obtained. In the above-described culture method, the yield may sometimes vary depending on the sequence of the antibody, and therefore, it is possible to select an antibody which is easily produced as a pharmaceutical by using the yield as an index among the antibodies having an equivalent binding activity. Therefore, in the antibody of the present invention, an antibody obtained by a method of producing an antibody, characterized by including a step of culturing the transformed host cell and a step of collecting a desired antibody from a cultured product obtained in the culturing step is also included.

It is known that a lysine residue at the carboxyl terminus of the heavy chain of an antibody produced in a cultured mammalian cell is deleted (Journal of Chromatography A, 705: 129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360: 75-83 (2007)). However, such deletion and modification of the heavy chain sequence do not affect the antigen-binding affinity and the effector function (the activation of a complement, the antibody-dependent cellular cytotoxicity, etc.) of the antibody. Therefore, in the antibody according to the present invention, an antibody subjected to such modification and a functional fragment of the antibody are also included, and a deletion variant in which one or two amino acids have been deleted at the carboxyl terminus of the heavy chain, a variant obtained by amidation of the deletion variant (for example, a heavy chain in which the carboxyl terminal proline residue has been amidated), and the like are also encompassed. The type of deletion variant having a deletion at the carboxyl terminus of the heavy chain of the antibody according to the invention is not limited to the above variants as long as the antigen-binding affinity and the effector function are conserved. The two heavy chains constituting the antibody according to the invention may be of one type selected from the group consisting of a full-length heavy chain and the above-described deletion variant, or may be of two types in combination selected therefrom. The ratio of the amount of each deletion variant can be affected by the type of cultured mammalian cells which produce the antibody according to the invention and the culture conditions, however, a case where one amino acid residue at the carboxyl terminus has been deleted in both of the two heavy chains contained as main components in the antibody according to the invention can be exemplified.

As isotype of the antibody of the invention, for example, IgG (IgG1, IgG2, IgG3, IgG4) can be exemplified, and IgG1 or IgG2 can be exemplified preferably.

As the biological activity of the antibody, generally an antigen-binding activity, an activity of internalizing in cells expressing an antigen by binding to the antigen, an activity of neutralizing the activity of an antigen, an activity of enhancing the activity of an antigen, an antibody-dependent cellular cytotoxicity (ADCC) activity, a complement-dependent cytotoxicity (CDC) activity, and an antibody-dependent cell-mediated phagocytosis (ADCP) activity can be exemplified. The function of the antibody of the present invention is a binding activity to TROP2, preferably an activity of internalizing in TROP2-expressing cells by binding to TROP2. Further, the antibody of the present invention may have an ADCC activity, a CDC activity, and/or an ADCP activity in addition to a cell internalization activity.

The obtained antibody can be purified to homogeneity. The separation and purification of the antibody may be performed employing a conventional protein separation and purification method. For example, the antibody can be separated and purified by appropriately selecting and combining column chromatography, filter filtration, ultrafiltration, salt precipitation, dialysis, preparative polyacrylamide gel electrophoresis, isoelectric focusing electrophoresis, and the like (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988)), but the method is not limited thereto.

Examples of such chromatography include affinity chromatography, ion exchange chromatography, hydrophobic chromatography, gel filtration chromatography, reverse phase chromatography, and adsorption chromatography.

Such chromatography can be performed employing liquid chromatography such as HPLC or FPLC.

As a column to be used in affinity chromatography, a Protein A column and a Protein G column can be exemplified. For example, as a column using a Protein A column, Hyper D, POROS, Sepharose FF (Pharmacia) and the like can be exemplified.

Further, by using a carrier having an antigen immobilized thereon, the antibody can also be purified utilizing the binding property of the antibody to the antigen.

### Anticancer Compound

The antitumor compound to be conjugated to the anti-TROP2 antibody as part of the disclosed antibody-drug conjugate of the present invention is explained in this section.

The antitumor compound used in the present invention is not particularly limited if it is a compound having an antitumor effect and a substituent group or a partial structure allowing connecting to a linker structure. When a part or whole linker is cleaved in tumor cells, the antitumor compound moiety is released to exhibit the antitumor effect of the antitumor compound. As the linker is cleaved at a connecting position to drug, the antitumor compound is released in its unmodified structure to exhibit its intrinsic antitumor effect.

As the antitumor compound used in the present invention, exatecan (((1S,9S)-1-amino-9-ethyl-5-fluoro-2,3-dihydro-9-hydroxy-4-methyl-1H,12H-benzo[de]pyrano[3',4':6,7]indolizino[1,2-b]quinoline-10,13(9H,15H)-dione; shown in the following formula), one of the camptothecin derivatives, can be preferably used. Exatecan is shown below in Formula 1.

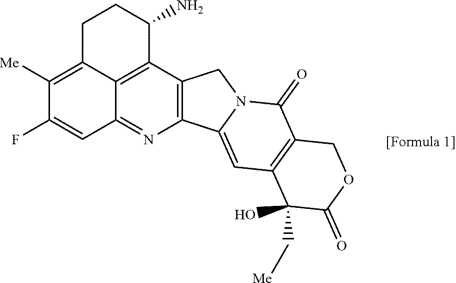 [SMILES: CC[C@@]1(O)C(=O)OCc2c1cc1n(c2=O)Cc2c-1nc1cc(F)c(C)c3c1c2[C@@H](N)CC3]

Although having an excellent antitumor effect, exatecan has not been commercialized as an antitumor drug. The compound can be easily obtained by a known method and the amino group at position 1 can be preferably used as a connecting position to the linker structure. Further, exatecan can also be released in tumor cells while part of the linker is still attached thereto, and it remains an excellent anticancer compound, exhibiting an excellent antitumor effect even in such structure.

Because exatecan has a camptothecin structure, it is known that the equilibrium shifts to a structure with a closed lactone ring (closed ring) in an aqueous acidic medium (for example, pH 3 or so) but it shifts to a structure with an open lactone ring (open ring) in an aqueous basic medium (for example, pH 10 or so). A drug conjugate being introduced with an exatecan residue corresponding to the closed ring structure and the open ring structure is also expected to have the same antitumor effect and any of these states is within the scope of the present invention.

Other examples of the antitumor compound can include doxorubicin, daunorubicin, mitomycin C, bleomycin, cyclocytidine, vincristine, vinblastine, methotrexate, platinum-based antitumor agent (cisplatin or derivatives thereof), taxol or derivatives thereof, and camptothecin or derivatives thereof (antitumor agent described in Japanese Patent Laid-Open No. 6-87746).

With regard to the antibody-drug conjugate, the number of conjugated drug molecules per antibody molecule is a key factor having an influence on the efficacy and safety. Production of the antibody-drug conjugate is performed by defining the reaction condition including the amounts of use of raw materials and reagents for reaction so as to have a constant number of conjugated drug molecules, and the antibody-drug conjugate is generally obtained as a mixture containing different numbers of conjugated drug molecules, unlike the chemical reaction of a low-molecular-weight compound. The number of drugs conjugated in an antibody molecule is expressed or specified by the average value, that is, the average number of conjugated drug molecules. Unless specifically described otherwise as a principle, the number of conjugated drug molecules means an average value except in a case in which it represents an antibody-drug conjugate having a specific number of conjugated drug molecules that is included in an antibody-drug conjugate mixture having different number of conjugated drug molecules. The number of exatecan molecules conjugated to an antibody molecule is controllable, and as an average number of conjugated drug molecules per antibody, about 1 to 10 exatecans can be connected. In some embodiment, 1, 2, 3, 4, 5, 6, 7, 8, 9, or 10 exatecans can be connected. Preferably, it is 2 to 8, more preferably 3 to 8, and more preferably 3.5 to 4.5, or 4. Meanwhile, a person skilled in the art can design a reaction for conjugating a required number of drug molecules to an antibody molecule based on the description of the Examples of the present application and can obtain an antibody-drug conjugate with a controlled number of exatecan molecules.

### Linker Structure

With regard to the anti-TROP2 antibody-drug conjugate of the present invention, the linker structure for conjugating an antitumor compound to the anti-TROP2 antibody is explained. The linker has a structure of the following formula:

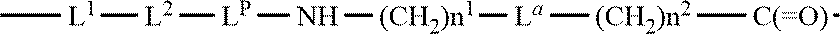

The antibody is connected to the terminal of L¹ (terminal opposite to the connection to L²), and the antitumor compound is connected to the carbonyl group of the —L^{a}—(CH₂)n²—C(═O)— moiety.

n¹ represents an integer of 0 to 6 and is preferably an integer of 1 to 5, and more preferably 1 to 3.

### L¹

L¹ is represented by the structure of

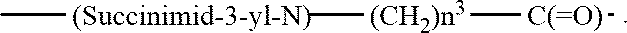

In the above, n³ is an integer of 2 to 8, and “-(Succinimid-3-yl-N)-” has a structure represented by the following formula:

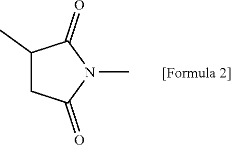

Position 3 of the above partial structure is a connecting position to the anti-TROP2 antibody. The bond to the anti-TROP2 antibody at position 3 is characterized by bonding with thioether formation. The nitrogen atom at position 1 of the structure moiety is connected to the carbon atom of methylene which is present within the linker including the structure. Specifically, -(Succinimid-3-yl-N)—(CH₂)n³—C(═O)—L²— is a structure represented by the following formula (herein, “antibody-S-” originates from an antibody).

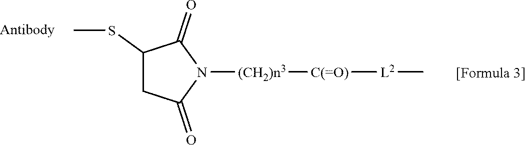

In the formula, n³ is an integer of 2 to 8, and preferably 2 to 5.

Specific examples of L¹ can include

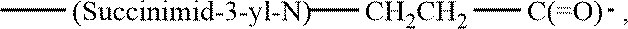

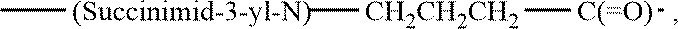

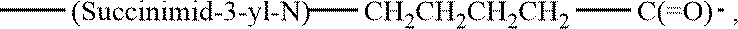

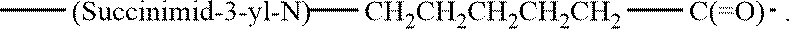

### L²

L² is a linker represented by the following structure:

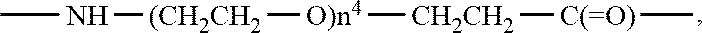

L² may not be present, and in such a case, L² is a single bond. In the above, n⁴ is an integer of 1 to 6, and preferably 2 to 4. L² is connected to L¹ at its terminal amino group and is connected to L^{P} at its carbonyl group at the other terminal.

Specific examples of L² can include

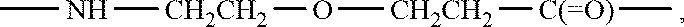

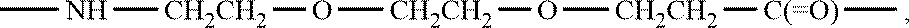

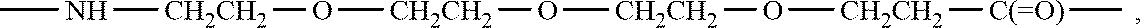

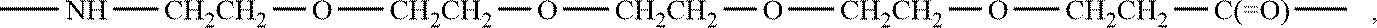

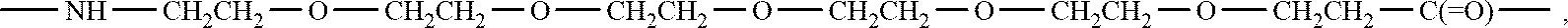

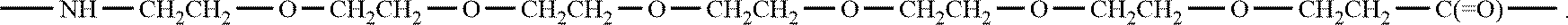

### L^{P}

L^{P} is a peptide residue consisting of 2 to 7 amino acids. Specifically, it consists of an oligopeptide residue in which 2 to 7 amino acids are linked by a peptide bonding. L^{P} is connected to L² at its N terminal and is connected to the amino group of —NH—(CH₂)n¹—L^{a}—(CH₂)n²—C(═O)—moiety of the linker at its C terminal.

The amino acid constituting L^{P} in the linker is not particularly limited, however, examples thereof include an L- or a D-amino acid, preferably an L-amino acid. And, it can be an amino acid having a structure such as β-alanine, ε-aminocaproic acid, or γ-aminobutyric acid in addition to an α-amino acid, further, it can be a non-natural type amino acid such as N-methylated amino acid.

The amino acid sequence of L^{P} is not particularly limited, but examples of the constituting amino acid include phenylalanine (Phe; F), tyrosine (Tyr; Y), leucine (Leu; L), glycine (Gly; G), alanine (Ala; A), valine (Val; V), lysine (Lys; K), citrulline (Cit), serine (Ser; S), glutamic acid (Glu; E), and aspartic acid (Asp; D).

Among them, preferred examples include phenylalanine, glycine, valine, lysine, citrulline, serine, glutamic acid, and aspartic acid. Depending on the type of the amino acid, drug release pattern can be controlled. The number of the amino acid can be between 2 to 7.

Specific examples of L^{P} can include

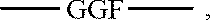

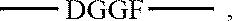

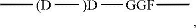

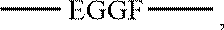

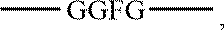

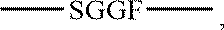

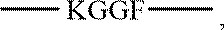

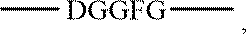

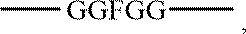

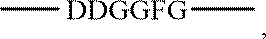

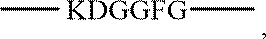

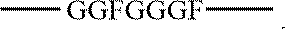

In the above, “(D-)D” represents a D-aspartic acid.

Particularly preferred examples of L^{P} for the antibody-drug conjugate of the present invention can include a tetrapeptide residue of -GGFG-.

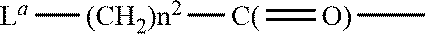

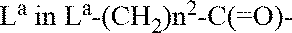

is a structure of —O— or a single bond. n² is an integer of 0 to 5, more preferably 0 to 3, more preferably 0 or 1.

Examples of L^{a}—(CH₂)n²—C(═O)— can include those having the following structures:

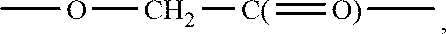

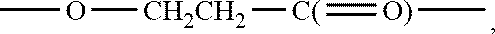

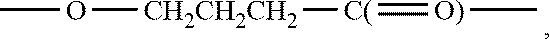

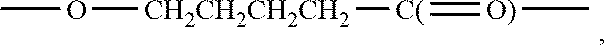

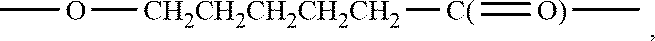

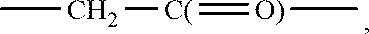

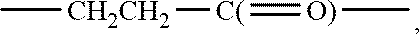

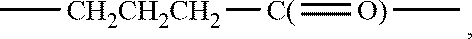

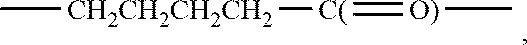

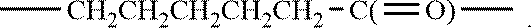

Of them, —O—CH₂—C(═O)—, —O—CH₂CH₂—C(═O)—, or a case in which L^{a} is a single bond, and n² is 0 is preferred.

Specific examples of the structure represented by —NH—(CH₂)n¹—L^{a}—(CH₂)n²—C(═O)— in the linker can include

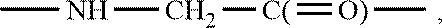

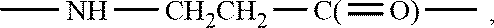

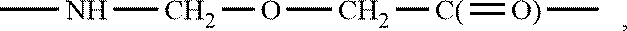

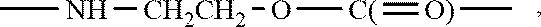

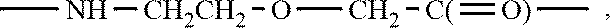

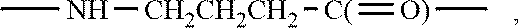

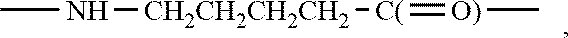

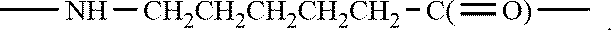

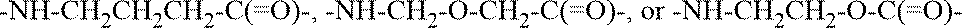

are preferrend.

In the linker, the chain length of —NH—(CH₂)n¹—L^{a}—(CH₂)n²—C(═O)— is preferably a chain length of 4 to 7 atoms, and more preferably a chain length of 5 or 6 atoms.

With regard to the anti-TROP2 antibody-drug conjugate of the present invention, it is considered that when the anti-TROP2 antibody-drug conjugate is transferred to the inside of tumor cells, the linker moiety is cleaved and the drug derivative having a structure represented by NH₂—(CH₂)n¹—L^{a}—(CH₂)n²—C(═O)—(NH—DX) is released to express an antitumor action. Examples of the antitumor derivative exhibiting an antitumor effect by releasing from the antibody-drug conjugate of the present invention include an antitumor derivative having a structure moiety in which the structure represented by —NH—(CH₂)n¹—L^{a}—(CH₂)n²—C(═O)— of the linker has a terminal amino group, and the particularly preferred include the followings.

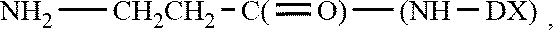

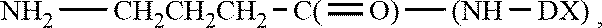

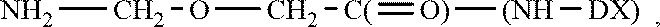

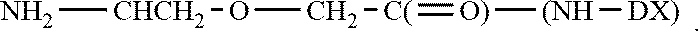

Meanwhile, in case of NH₂—CH₂—O—CH₂—C(═O)—(NH—DX), it was confirmed that, as the aminal structure in the molecule is unstable, it again undergoes a self-degradation to release the following:

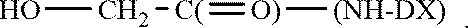

Those compounds can be also preferably used as a production intermediate of the antibody-drug conjugate of the present invention.

For the antibody-drug conjugate of the present invention in which exatecan is used as a drug, it is preferable that the drug-linker structure moiety [—L¹—L²—L^{P}—NH—(CH₂)n¹—L^{a}—(CH₂)n²—C(═O)—(NH—DX)] having the following structure is connected to an antibody. The average conjugated number of said drug-linker structure moiety per antibody can be 1 to 10, for example, 1, 2, 3, 4, 5, 6, 7, 8, 9, or 10. Preferably, it is 2 to 8, more preferably 3 to 8, and more preferably 3.5 to 4.5, or 4.

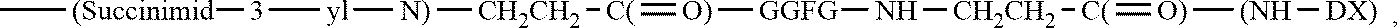

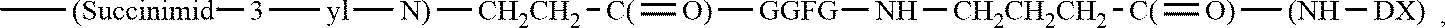

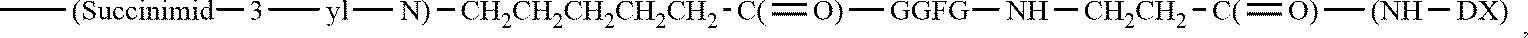

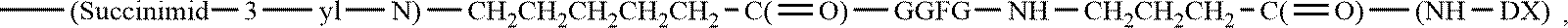

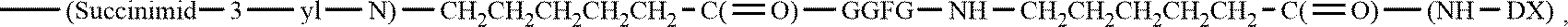

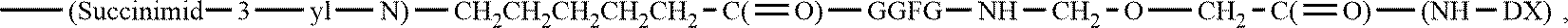

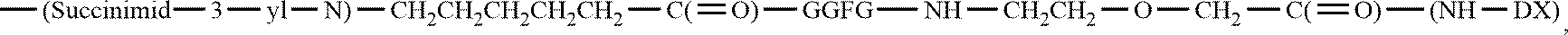

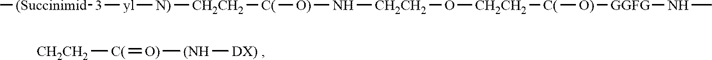

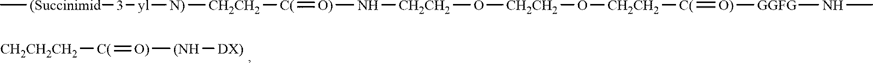

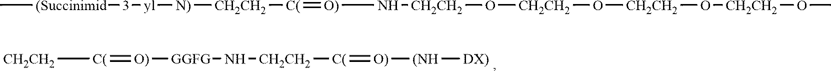

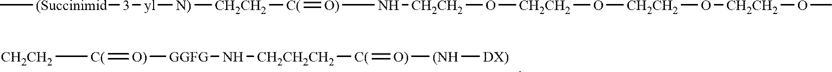

Among them, the more preferred are the following:

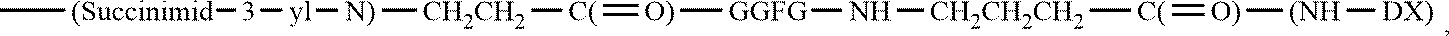

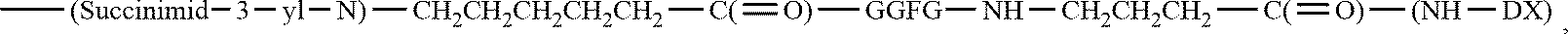

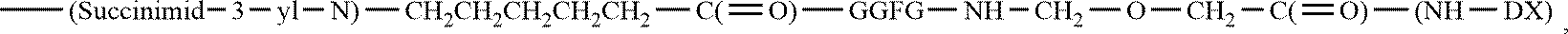

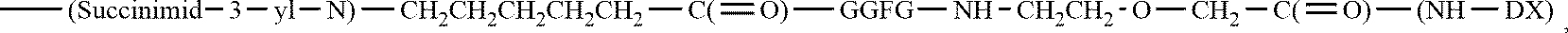

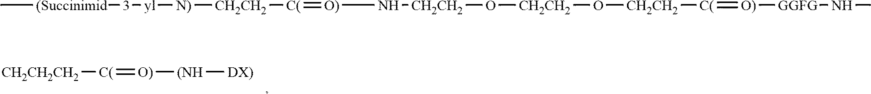

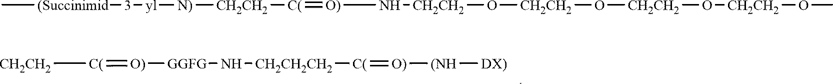

The particularly preferred are the following:

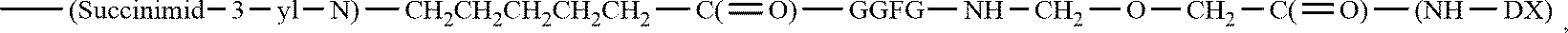

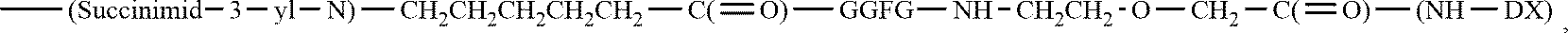

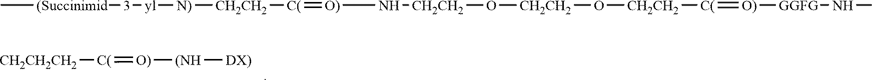

With regard to the linker structure for conjugating the anti-TROP2 antibody and a drug in the antibody-drug conjugate of the present invention, the preferred linker can be constructed by connecting preferred structures shown for each part of the linker explained above. As for the linker structure, those with the following structure can be preferably used. Meanwhile, the left terminal of the structure is a connecting position with the antibody and the right terminal is a connecting position with the drug.

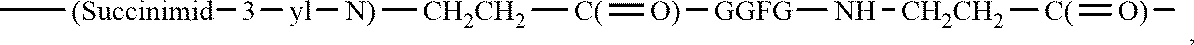

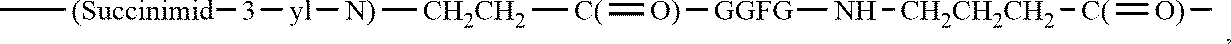

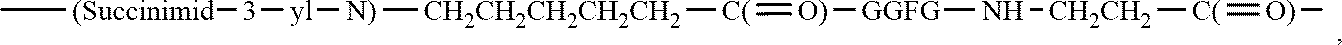

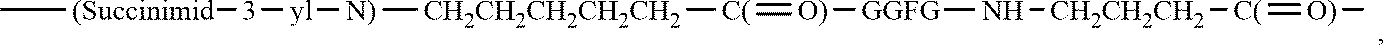

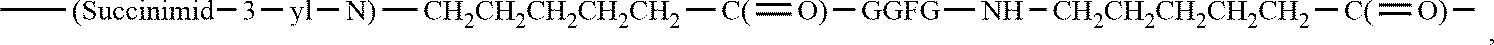

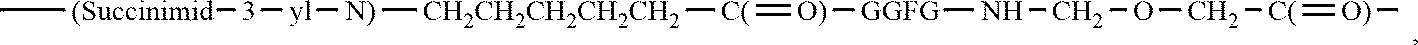

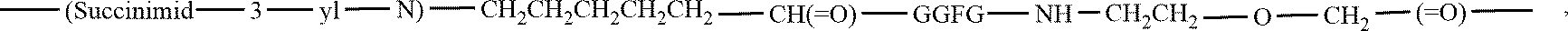

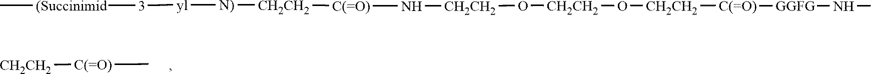

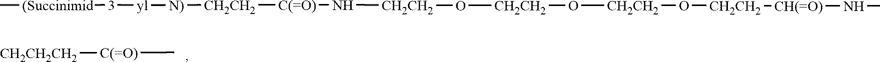

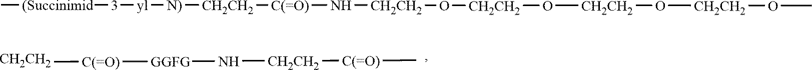

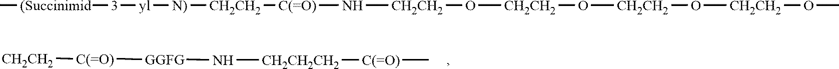

Among them, the more preferred are the following:

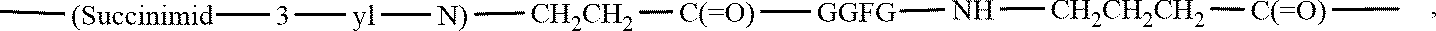

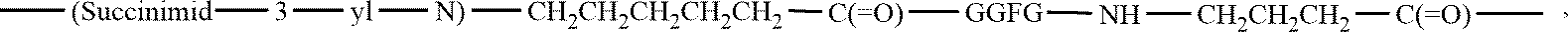

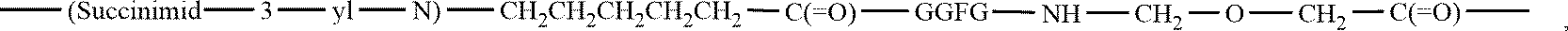

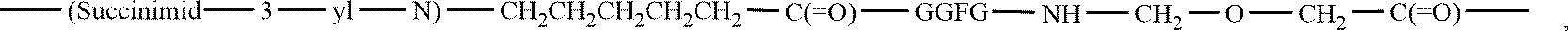

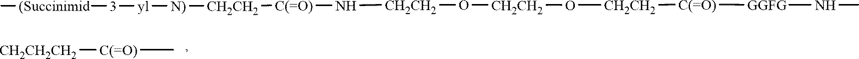

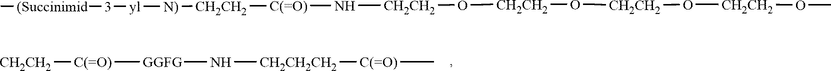

The particularly preferred include the following:

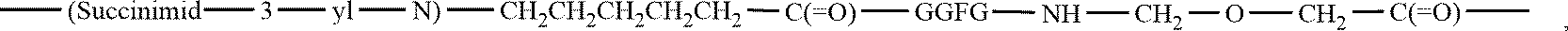

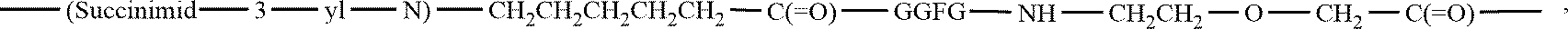

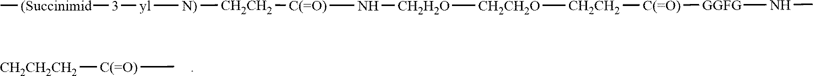

With regard to the anti-TROP2 antibody-drug conjugate used in the present invention, when it is transferred to the inside of tumor cells, the linker moiety is cleaved and the drug derivative having a structure represented by the formula:

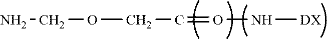

may be released. It has been confirmed that, as the aminal structure in the molecule of the drug derivative is unstable, it again undergoes a self-degradation to release a compound represented by the formula: HO—CH₂—C(═O)—(NH—DX).

The compound can be represented by the following formula:

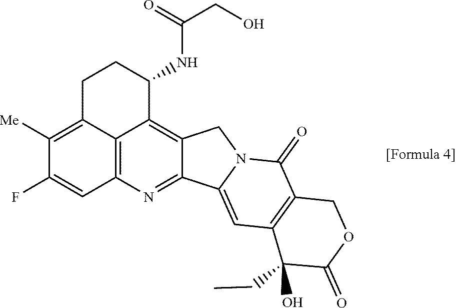 [SMILES: CC[C@@]1(O)C(=O)OCc2c1cc1n(c2=O)Cc2c-1nc1cc(F)c(C)c3c1c2[C@@H](NC(=O)CO)CC3]

(hereinafter, also referred to as “Compound 1” in the present invention).

Compound 1 is considered as the main pharmaceutically active substance of antitumor activity possessed by the antibody-drug conjugate used in the present invention and has been confirmed to have a topoisomerase I inhibitory effect (Ogitani Y. et al., Clinical Cancer Research, 2016, Oct 15; 22 (20):5097-5108, Epub 2016 Mar 29).

### Production Methods

Next, explanations are given for the representative method for producing the antibody-drug conjugate of the present invention or a production intermediate thereof. Meanwhile, the compounds are herein below described with the compound number shown in each reaction formula. Specifically, they are referred to as a “compound of the formula (1)”, a “compound (1)”, or the like. The compounds with numbers other than those are also described similarly.

### Production Method A

The antibody-drug conjugate represented by the formula (1) which is connected to the drug-linker structure via thioether can be produced by the following method, for example.

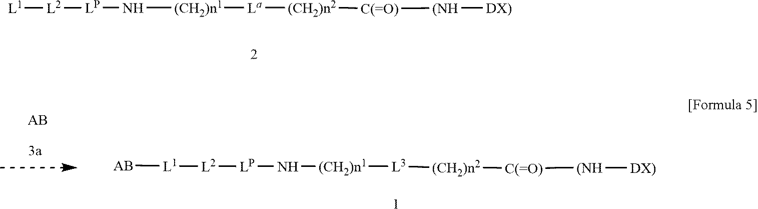

In the formula, AB represents an antibody having a sulfhydryl group, and L^{1'} represents L¹ linker structure in which the linker terminal is a maleimidyl group (formula shown below)

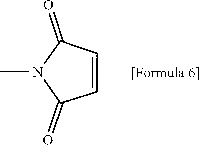

In the formula, the nitrogen atom is the connecting position, and specifically represents a group in which the -(Succinimid-3-yl—N)— moiety in -(Succinimid-3-yl—N)—(CH₂)n³—C(═O)— of L¹ is a maleimidyl group. Further, the —(NH—DX) represents a structure represented by the following formula:

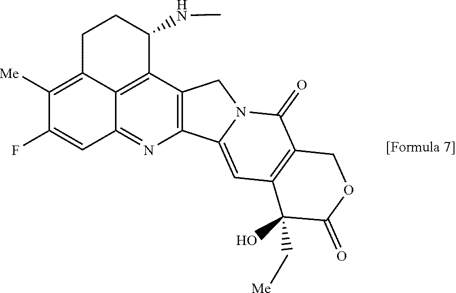 [SMILES: CC[C@@]1(O)C(=O)OCc2c1cc1n(c2=O)Cc2c-1nc1cc(F)c(C)c3c1c2[C@@H](NC)CC3]

and it represents a group that is derived by removing one hydrogen atom of the amino group at position 1 of exatecan.

Further, the compound of the formula (1) in the above reaction formula is interpreted as a structure in which one structure moiety corresponding from drug to the linker terminal connects to one antibody. However, it is only the description given for the sake of convenience, and there are actually many cases in which a plurality of the structure moieties are connected to one antibody molecule. The same applies to the explanation of the production method described below.

The antibody-drug conjugate (1) can be produced by reacting the compound (2), which is obtainable by the method described below, with the antibody (3a) having a sulfhydryl group.

The antibody (3a) having a sulfhydryl group can be obtained by a method well known in the art (Hermanson, G.T, Bioconjugate Techniques, pp. 56-136, pp. 456-493, Academic Press (1996)). Examples include: Traut’s reagent is reacted with the amino group of the antibody; N-succinimidyl S-acetylthioalkanoates are reacted with the amino group of the antibody followed by reaction with hydroxylamine; after reacting with N-succinimidyl 3-(pyridyldithio)propionate, the antibody is reacted with a reducing agent; the antibody is reacted with a reducing agent such as dithiothreitol, 2-mercaptoethanol, and tris(2-carboxyethyl)phosphine hydrochloride (TCEP) to reduce the disulfide bond in the antibody to form a sulfhydryl group, but it is not limited thereto.

Specifically, using 0.3 to 3 molar equivalents of TCEP as a reducing agent per disulfide in the antibody and reacting with the antibody in a buffer solution containing a chelating agent, the antibody with partially or completely reduced disulfide in the antibody can be obtained. Examples of the chelating agent include ethylenediamine tetraacetic acid (EDTA) and diethylenetriamine pentaacetic acid (DTPA). It can be used at concentration of 1 mM to 20 mM. Examples of the buffer solution which may be used include a solution of sodium phosphate, sodium borate, or sodium acetate. Specifically, by reacting the antibody with TCEP at 4° C. to 37° C. for 1 to 4 hours, the antibody (3a) having partially or completely reduced sulfhydryl group can be obtained.

Meanwhile, by conducting the reaction for adding a sulfhydryl group to a drug-linker moiety, the drug-linker moiety can be conjugated by a thioether bond.

Using 2 to 20 molar equivalents of the compound (2) per the antibody (3a) having a sulfhydryl group, the antibody-drug conjugate (1) in which 2 to 8 drug molecules are conjugated per antibody can be produced. Specifically, it is sufficient that the solution containing the compound (2) dissolved therein is added to a buffer solution containing the antibody (3a) having a sulfhydryl group for the reaction. Herein, examples of the buffer solution which may be used include sodium acetate solution, sodium phosphate, and sodium borate. pH for the reaction is 5 to 9, and more preferably the reaction is performed near pH 7. Examples of the solvent for dissolving the compound (2) include an organic solvent such as dimethyl sulfoxide (DMSO), dimethylformamide (DMF), dimethyl acetamide (DMA), and N-methyl-2-pyridone (NMP).

It is sufficient that the organic solvent solution containing the compound (2) dissolved therein is added at 1 to 20% v/v to a buffer solution containing the antibody (3a) having a sulfhydryl group for the reaction. The reaction temperature is 0 to 37° C., more preferably 10 to 25° C., and the reaction time is 0.5 to 2 hours. The reaction can be terminated by deactivating the reactivity of unreacted compound (2) with a thiol-containing reagent. Examples of the thiol-containing reagent include cysteine and N-acetyl-L-cysteine (NAC). More specifically, 1 to 2 molar equivalents of NAC are added to the compound (2) used and, by incubating at room temperature for 10 to 30 minutes, the reaction can be terminated.

The produced antibody-drug conjugate (1) can be subjected to, after concentration, buffer exchange, purification, and measurement of antibody concentration and average number of conjugated drug molecules per antibody molecule according to common procedures described below, identification of the antibody-drug conjugate (1).

### Common Procedure A: Concentration of Aqueous Solution of Antibody or Antibody-Drug Conjugate

To a Amicon Ultra (50,000 MWCO, Millipore Corporation) container, a solution of antibody or antibody-drug conjugate was added and the solution of the antibody or antibody-drug conjugate was concentrated by centrifugation (centrifuge for 5 to 20 minutes at 2000 G to 3800 G) using a centrifuge (Allegra X-15R, Beckman Coulter, Inc.).

### Common Procedure B: Measurement of Antibody Concentration

Using a UV detector (Nanodrop 1000, Thermo Fisher Scientific Inc.), measurement of the antibody concentration was performed according to the method defined by the manufacturer. At that time, 280 nm absorption coefficient different for each antibody was used (1.3 mLmg⁻¹cm⁻¹ to 1.8 mLmg⁻¹cm⁻¹).

### Common Procedure C-1: Buffer Exchange for Antibody

NAP-25 column (Cat. No. 17-0852-02, GE Healthcare Japan Corporation) using Sephadex G-25 carrier was equilibrated with phosphate buffer (10 mM, pH 6.0; it is referred to as PBS6.0/EDTA in the specification) containing sodium chloride (137 mM) and ethylene diamine tetraacetic acid (EDTA, 5 mM) according to the method defined by the manufacturer. Aqueous solution of the antibody was applied in an amount of 2.5 mL to single NAP-25 column, and then the fraction (3.5 mL) eluted with 3.5 mL of PBS6.0/EDTA was collected. The resulting fraction was concentrated by the Common procedure A. After measuring the concentration of the antibody using the Common procedure B, the antibody concentration was adjusted to 10 mg/mL using PBS6.0/EDTA.

### Common Procedure C-2: Buffer Exchange for Antibody

NAP-25 column (Cat. No. 17-0852-02, GE Healthcare Japan Corporation) using Sephadex G-25 carrier was equilibrated with phosphate buffer (50 mM, pH 6.5; it is referred to as PBS6.5/EDTA in the specification) containing sodium chloride (50 mM) and EDTA (2 mM) according to the method defined by the manufacturer. Aqueous solution of the antibody was applied in an amount of 2.5 mL to single NAP-25 column, and then the fraction (3.5 mL) eluted with 3.5 mL of PBS6.5/EDTA was collected. The resulting fraction was concentrated by the Common procedure A. After measuring the concentration of the antibody using the Common procedure B, the antibody concentration was adjusted to 20 mg/mL using PBS6.5/EDTA.

### Common Procedure D: Purification of Antibody-Drug Conjugate

NAP-25 column was equilibrated with any buffer selected from commercially available phosphate buffer (PBS7.4, Cat. No. 10010-023, Invitrogen), sodium phosphate buffer (10 mM, pH 6.0; it is referred to as PBS6.0) containing sodium chloride (137 mM), and acetate buffer containing sorbitol (5%) (10 mM, pH 5.5; it is referred to as ABS in the specification). Aqueous solution of the antibody-drug conjugate reaction was applied in an amount of about 1.5 mL to the NAP-25 column, and then eluted with the buffer in an amount defined by the manufacturer to collect the antibody fraction. The collected fraction was again applied to the NAP-25 column and, by repeating 2 to 3 times in total the gel filtration purification process for eluting with buffer, the antibody-drug conjugate excluding non-conjugated drug linker and a low-molecular-weight compound (tris(2-carboxyethyl)phosphine hydrochloride (TCEP), N-acetyl-L-cysteine (NAC), and dimethyl sulfoxide) was obtained.

### Common Procedure E: Measurement of Antibody Concentration in Antibody-Drug CONJUGATE and Average Number of Conjugated Drug Molecules Per Antibody Molecule (1)

The conjugated drug concentration in the antibody-drug conjugate can be calculated by measuring UV absorbance of an aqueous solution of the antibody-drug conjugate at two wavelengths of 280 nm and 370 nm, followed by performing the calculation shown below.

Because the total absorbance at any wavelength is equal to the sum of the absorbance of every light-absorbing chemical species that are present in a system (additivity of absorbance), when the molar absorption coefficients of the antibody and the drug remain the same before and after conjugation between the antibody and the drug, the antibody concentration and the drug concentration in the antibody-drug conjugate are expressed with the following equations.

$${\text{A}}_{280} = {\text{A}}_{\text{D} , 280} + {\text{A}}_{\text{A} , 280} \text{=} {\text{ε}}_{\text{D} , 280} {\text{C}}_{\text{D}} + {\text{ε}}_{\text{A} , 280} {\text{C}}_{\text{A}} \qquad \text{Equation (I)}$$

$${\text{A}}_{370} = {\text{A}}_{\text{D} , 370} + {\text{A}}_{\text{A} , 370} = {\text{ε}}_{\text{D} , 370} {\text{C}}_{\text{D} , 370} + {\text{ε}}_{\text{A} , 370} {\text{C}}_{\text{A}} \qquad \text{Equation (II)}$$

In the above, A₂₈₀ represents the absorbance of an aqueous solution of the antibody-drug conjugate at 280 nm, A₃₇₀ represents the absorbance of an aqueous solution of the antibody-drug conjugate at 370 nm, A_{A,280} represents the absorbance of an antibody at 280 nm, A_{A,370} represents the absorbance of an antibody at 370 nm, A_{D,280} represents the absorbance of a conjugate precursor at 280 nm, A_{D,370} represents the absorbance of a conjugate precursor at 370 nm, ε_{A,280} represents the molar absorption coefficient of an antibody at 280 nm, ε_{A,370} represents the molar absorption coefficient of an antibody at 370 nm, ε_{D,280} represents the molar absorption coefficient of a conjugate precursor at 280 nm, ε_{D,370} represents the molar absorption coefficient of a conjugate precursor at 370 nm, C_{A} represents the antibody concentration in an antibody-drug conjugate, and C_{D} represent the drug concentration in an antibody-drug conjugate.

As for ε_{A,280}, ε_{A,370}, ε_{D,280}, and ε_{D,370} in the above, previously prepared values (estimated value based on calculation or measurement value obtained by UV measurement of the compound) are used. For example, ε_{A,280} can be estimated from the amino acid sequence of an antibody using a known calculation method (Protein Science, 1995, vol. 4, 2411-2423). ε_{A,370} is generally zero. ε_{D,280} and ε_{D,370} can be obtained based on Lambert-Beer’s law (Absorbance = molar concentration × molar absorption coefficient × cell path length) by measuring the absorbance of a solution in which the conjugate precursor to be used is dissolved at a certain molar concentration. By measuring A₂₈₀ and A₃₇₀ of an aqueous solution of the antibody-drug conjugate and solving the simultaneous equations (I) and (II) using the values, C_{A} and C_{D} can be obtained. Further, by diving C_{D} by C_{A}, the average number of conjugated drug per antibody can be obtained.

### Common Procedure F: Measurement of Average Number of Conjugated Drug Molecules Per Antibody Molecule in Antibody-Drug Conjugate - (2)

The average number of conjugated drug molecules per antibody molecule in the antibody-drug conjugate can be also determined by high-performance liquid chromatography (HPLC) analysis using a method described below, in addition to the above-mentioned Common procedure E.

### F-1. Preparation of Sample for HPLC Analysis (Reduction of Antibody-Drug Conjugate)

An antibody-drug conjugate solution (about 1 mg/mL, 60 µL) is mixed with an aqueous dithiothreitol (DTT) solution (100 mM, 15 µL). The mixture is incubated at 37° C. for 30 minutes to cleave the disulfide bond between the L chain and the H chain of the antibody-drug conjugate. The resulting sample is used in HPLC analysis.

### F-2. HPLC Analysis

The HPLC analysis is conducted under the following measurement conditions:
HPLC system: Agilent 1290 HPLC system (Agilent Technologies, Inc.)
Detector: UV absorption spectrometer (measurement wavelength: 280 nm)
Column: PLRP-S (2.1 × 50 mm, 8 µm, 1000 angstroms; Agilent Technologies, Inc., P/N PL1912-1802)
Column temperature: 80° C.
Mobile phase A: 0.04% aqueous trifluoroacetic acid (TFA) solution
Mobile phase B: acetonitrile solution containing 0.04% TFA
Gradient program: 29%-36% (0 min-12.5 min), 36%-42% (12.5-15 min), 42%-29% (15 min-15.1 min), 29%-29% (15.1 min-25 min)
Sample injection volume: 15 µL

### F-3. Data Analysis

[F-3-1] Compared with an L chain (L₀) and an H chain (H₀) of a non-conjugated antibody, a drug-conjugated L chain (L chain connected to one drug molecule: L₁) and H chains (H chain connected to one drug molecule: H₁, H chain connected to two drug molecule: H₂, H chain connected to three drug molecules: H₃) exhibit higher hydrophobicity in proportion to the number of conjugated drug molecules and thus have a larger retention time. These chains are therefore eluted in the order of L₀ and L₁ or H₀, H₁, H₂, and H₃. Detection peaks can be assigned to any of L₀, L₁, H₀, H₁, H₂, and H₃ by the comparison of retention times with L₀ and H₀.

[F-3-2] Since the drug linker has UV absorption, peak area values are corrected in response to the number of conjugated drug linker molecules according to the following expression using the molar absorption coefficients of the L chain, the H chain, and the drug linker.

$$\begin{matrix}\text{Corrected value of the peak area of the L chain} \left({L}_{i}\right) \\ = \text{Peak area} \\ \times \frac{\text{Molar extinction coefficient of the L chain}}{\begin{matrix}\text{Molar extiction coefficient of the L chain + the number of conjugated drug molecules} \times \text{Molar extinction} \\ \text{coefficient of the drug linker}\end{matrix}}\end{matrix} \qquad \text{[Expression 1]}$$

$$\begin{matrix}\text{Corrected value of the peak area of the H chain} \left({H}_{i}\right) \\ = \text{Peak area} \\ \times \frac{\text{Molar extinction coefficient of the H chain}}{\begin{matrix}\text{Molar extinction coefficient of the H chain + the number of conjugated drug molecules} \times \\ \text{Molar extinction coefficient of the drug linker}\end{matrix}}\end{matrix} \qquad \text{[Expression 2]}$$

Here, as for the molar extinction coefficient (280 nm) of the L chain or the H chain of each antibody, a value estimated from the amino acid sequence of the L chain or the H chain of each antibody by a known calculation method (Protein Science, 1995, vol. 4, 2411-2423) can be used. In the case of hTINA, a molar extinctio coefficient of 34690 and a molar extinctio coefficient of 95000 were used as estimated values for the L chain and the H chain, respectively, according to its amino acid sequence. As for the molar extinctio coefficient (280 nm) of the drug linker, the measured molar extinctio coefficient (280 nm) of a compound in which the maleimide group was converted to succinimide thioether by the reaction of each drug linker with mercaptoethanol or N-acetylcysteine was used.

[F-3-3] The peak area ratio (%) of each chain is calculated for the total of the corrected values of peak areas according to the following expression.

$$\begin{matrix}\text{Peak area ratio of the L chain} = \frac{{A}_{L i}}{{A}_{L 0} + {A}_{L 1}} \times 100 \\ \text{Peak area ratio of the H chain} = \frac{{A}_{H i}}{{A}_{H 0} + {A}_{H 1} + {A}_{H 2} + {A}_{H 3}} \times 100 \\ \begin{matrix}{A}_{L i} , {A}_{H i} : \text{Corrected values of respective peak} \\ \text{areas of} {L}_{i} \text{and} {H}_{i}\end{matrix}\end{matrix} \qquad \text{[Expression 3]}$$

[F-3-4] The average number of conjugated drug molecules per antibody molecule in the antibody-drug conjugate is calculated according to the following expression.

Average number of conjugated drug molecules = (L₀ peak area ratio × 0 + L₀ peak area ratio × 1 + H₀ peak area ratio × 0 + H₁ peak area ratio × 1 + H₂ peak area ratio × 2 + H₃ peak area ratio × 3) / 100 × 2.

The compound represented by the formula (2) in Production method 1 is a compound represented by the following formula:

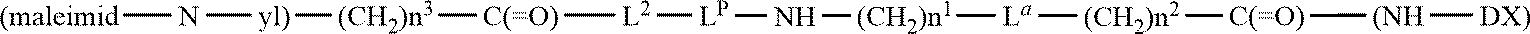

In the formula,
n³ represents an integer of 2 to 8,
L² represents —NH—(CH₂CH₂—O)n⁴—CH₂CH₂—C(═O)— or a single bond, wherein n⁴ represents an integer of 1 to 6,
L^{P} represents a peptide residue consisting of 2 to 7 amino acids selected from phenylalanine, glycine, valine, lysine, citrulline, serine, glutamic acid, and aspartic acid
n¹ represents an integer of 0 to 6,
n² represents an integer of 0 to 5,
L^{a} represents —O— or a single bond,
(maleimid-N-yl)- is a maleimidyl group (2,5-dioxo-2,5-dihydro-1H-pyrrol-1-yl group) represented by the following formula:

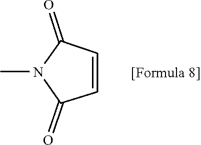

wherein the nitrogen atom is a connecting position,
—(NH—DX) is a group represented by the following formula:

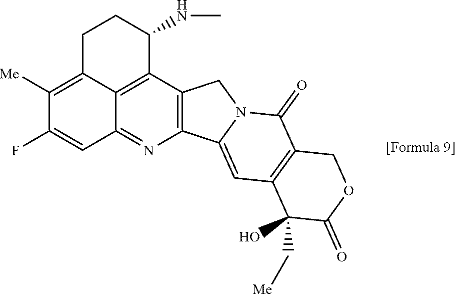 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is a connecting position.

When L² is a single bond or —NH—(CH₂CH₂—O)n⁴—CH₂CH₂—C(═O)—, a compound in which n⁴ is an integer of 2 to 4 is preferred as a production intermediate.

As for the peptide residue of L^{P}, a compound having a peptide residue comprising an amino acid selected from phenylalanine, glycine, valine, lysine, citrulline, serine, glutamic acid, and aspartic acid is preferred as a production intermediate. Among those peptide residues, a compound in which L^{P} is a peptide residue consisting of 4 amino acids is preferred as a production intermediate. More specifically, a compound in which L^{P} is a tetrapeptide residue of -GGFG- is preferred as a production intermediate.

Further, as for the —NH—(CH₂)n¹—L^{a}—(CH₂)n²—, a compound having —NH—CH₂CH₂—, —NH—CH₂CH₂CH₂—, —NH—CH₂CH₂CH₂CH₂—, —NH—CH₂CH₂CH₂CH₂CH₂—, —NH—CH₂—O—CH₂—, or —NH—CH₂CH₂—O—CH₂— is preferred as a production intermediate. A compound having —NH—CH₂CH₂CH₂—, —NH—CH₂—O—CH₂—, or —NH—CH₂CH₂—O—CH₂ is more preferred.

Further, in the compound represented by the formula (2), a compound in which n³ is an integer of 2 to 5, L² is a single bond, and —NH—(CH₂)n¹—L^{a}—(CH₂)n²— is —NH—CH₂CH₂—, —NH—CH₂CH₂CH₂—, —NH—CH₂CH₂CH₂CH₂—, —NH—CH₂CH₂CH₂CH₂CH₂—, —NH—CH₂—O—CH₂—, or —NH—CH₂CH₂—O—CH₂— is preferred as a production intermediate. A compound in which —NH—(CH₂)n¹—L^{a}—(CH₂)n²— is —NH—CH₂CH₂—, —NH—CH₂CH₂CH₂—, —NH—CH₂—O—CH₂—, or —NH—CH₂CH₂—O—CH₂— is more preferred. A compound in which n³ is an integer of 2 or 5 is further preferred.

Further, in the compound represented by the formula (2), a compound in which n³ is an integer of 2 to 5, L² is —NH—(CH₂CH₂—O)n⁴—CH₂CH₂—C(═O)—, n⁴ is an integer of 2 to 4, and —NH—(CH₂)n¹—L^{a}—(CH₂)n²— is —NH—CH₂CH₂—, —NH—CH₂CH₂CH₂—, —NH—CH₂CH₂CH₂CH₂—, —NH—CH₂CH₂CH₂CH₂CH₂—, —NH—CH₂—O—CH₂—, or —NH—CH₂CH₂—O—CH₂— is preferred as a production intermediate. A compound in which n⁴ is an integer of 2 or 4 is more preferred. A compound in which —NH—(CH₂)n¹—L^{a}—(CH₂)n²— is —NH—CH₂CH₂CH₂—, —NH—CH₂—O—CH₂—, or —NH—CH₂CH₂—O—CH₂— is further preferred.

As such preferred intermediates useful in the production of the compound of the present invention, the followings can be exemplified.

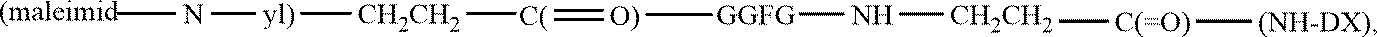

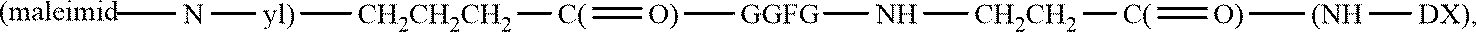

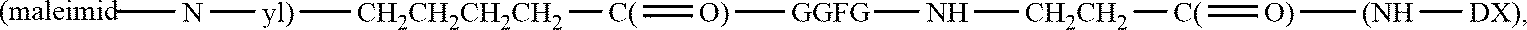

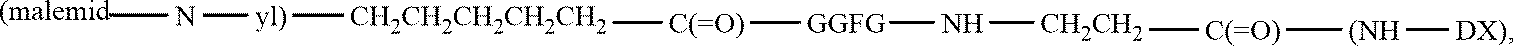

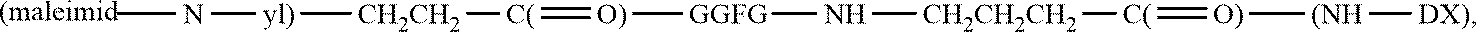

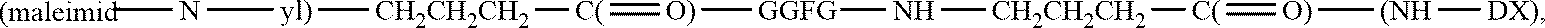

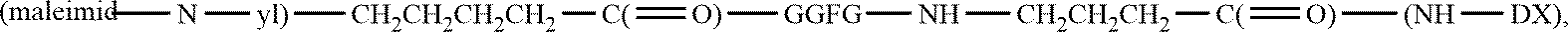

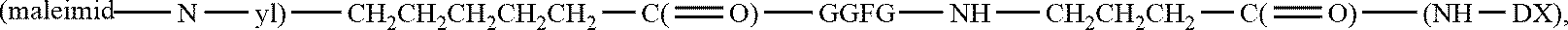

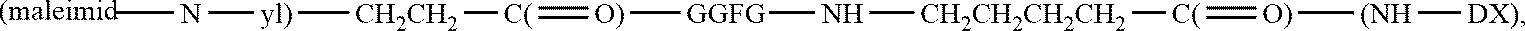

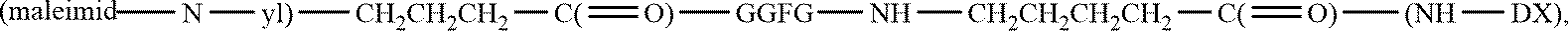

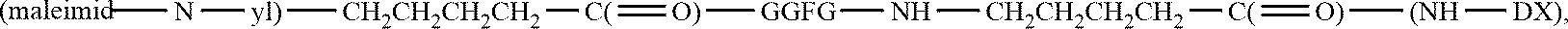

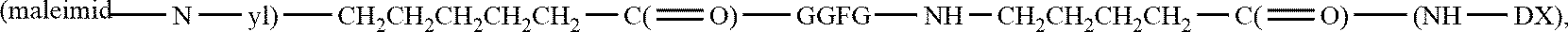

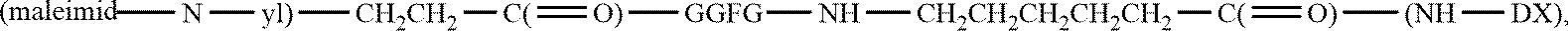

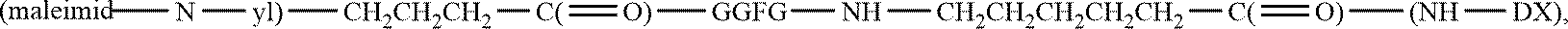

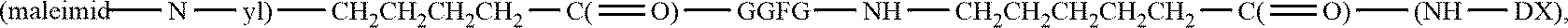

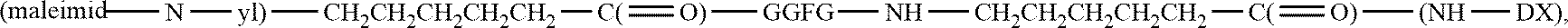

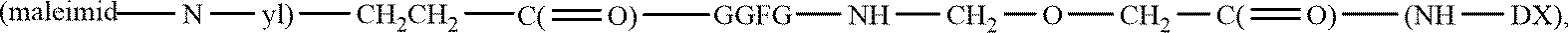

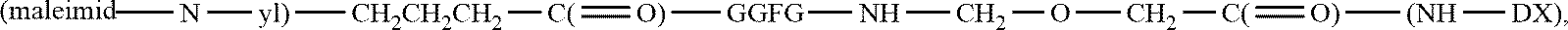

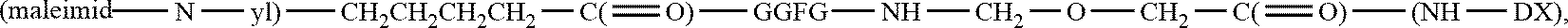

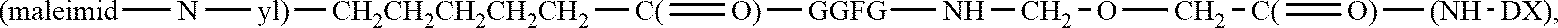

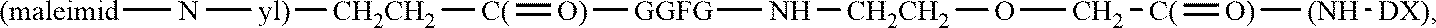

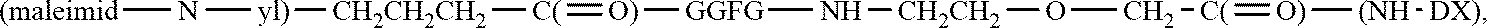

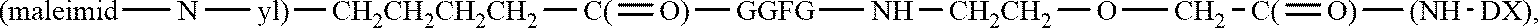

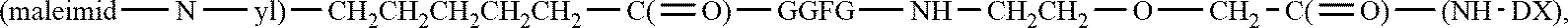

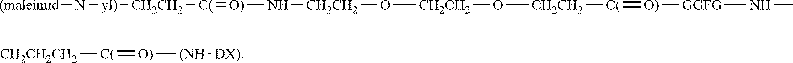

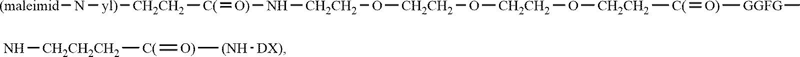

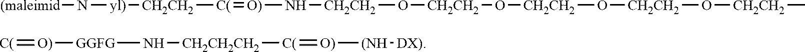

The anti-TROP2 antibody-drug conjugate of the present invention can be produced by reacting a drug-linker compound selected from the above-described group of production intermediate compounds with an anti-TROP2 antibody or a reactive derivative thereof and forming a thioether bond at a disulfide bond site present in the anti-TROP2 antibody. In this case, a reactive derivative of the anti-TROP2 antibody is preferably used. Particularly, a reactive derivative obtained by reducing the anti-TROP2 antibody is preferred.

The followings are compounds more preferred as production intermediates.

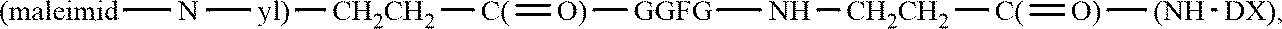

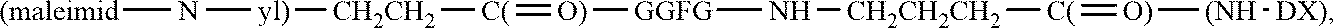

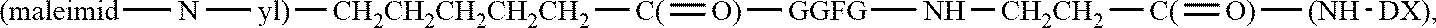

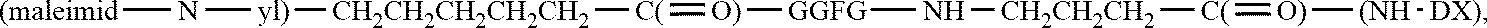

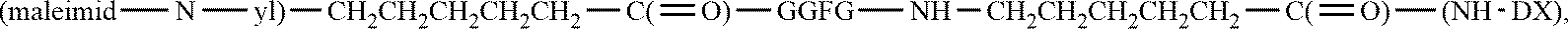

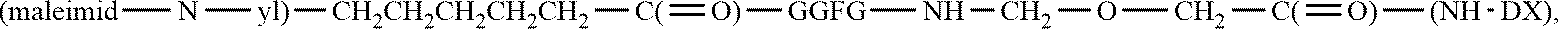

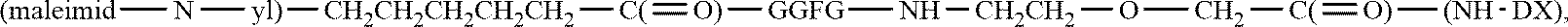

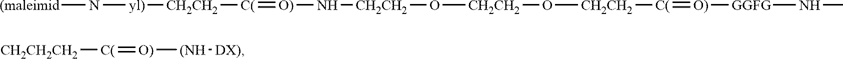

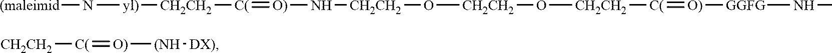

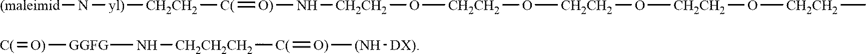

Among the above-described group of intermediate compounds, a compound represented by the following formula:

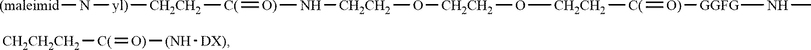

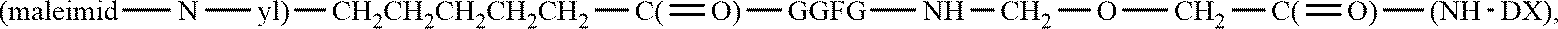

or

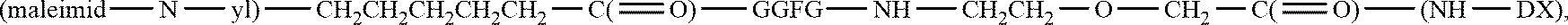

is a further preferred compound.

In order to secure the amount of the conjugate, a plurality of conjugates obtained under similar production conditions to have an equivalent number of drugs (e.g., about + 1) can be mixed to prepare new lots. In this case, the average number of drugs falls between the average numbers of drugs in the conjugates before the mixing.

### Production Method 2

The compound represented by the formula (2) as an intermediate used in the previous production method and a pharmacologically acceptable salt thereof can be produced by the following method, for example.

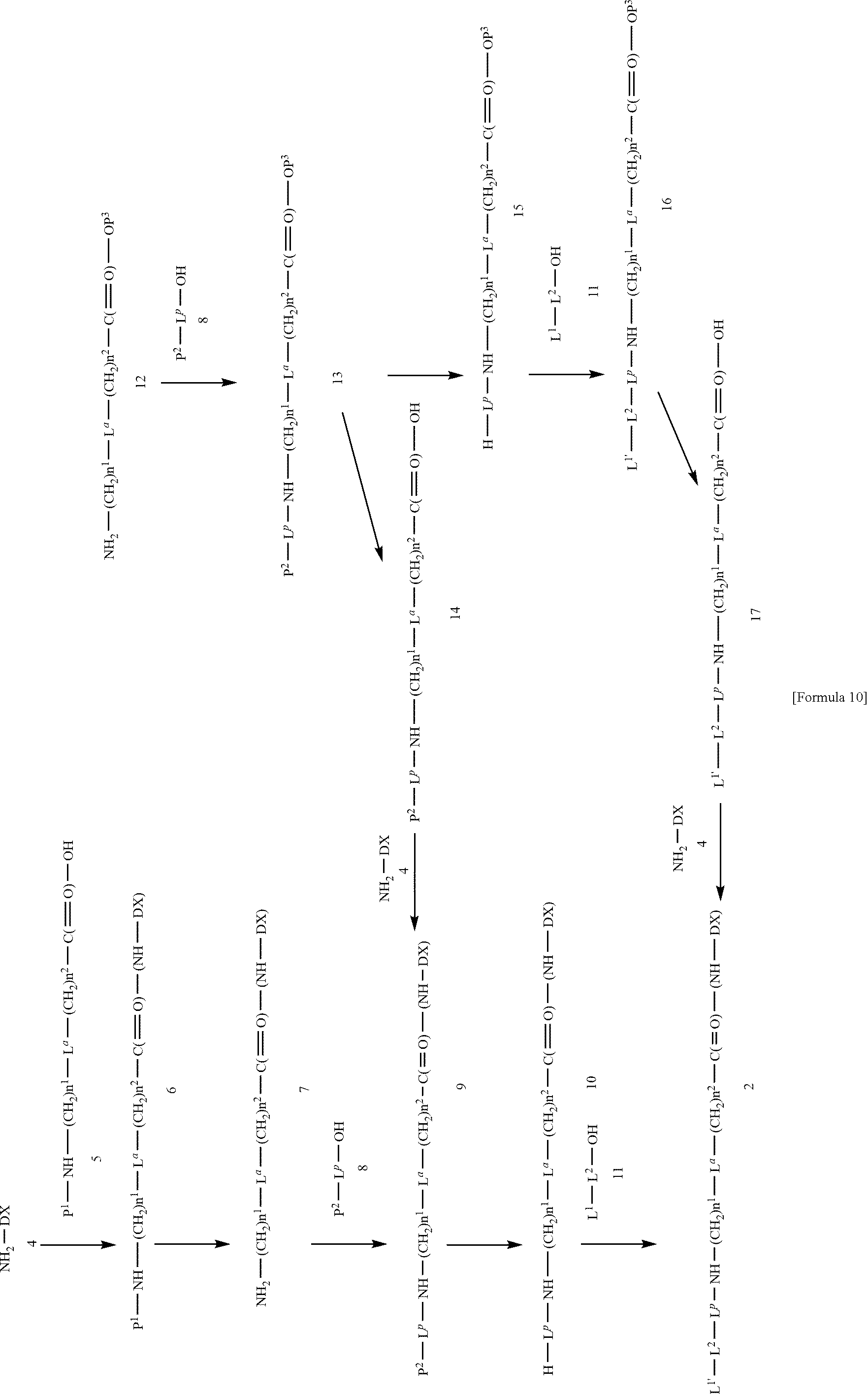

In the formula, L^{1'} represents a maleimidyl group, and P¹, P², and P³ each represents a protecting group.

The compound (6) can be produced by derivatizing the carboxylic acid (5) into an active ester, mixed acid anhydride, acid halide, or the like and reacting it with NH₂-DX (4) or a pharmacologically acceptable salt thereof in the presence of a base. NH₂-DX (4) represents exatecan (chemical name: (1S,9S)-1-amino-9-ethyl-5-fluoro-2,3-dihydro-9-hydroxy-4-methyl-1H,12H-benzo[de]pyrano[3',4':6,7]indolizino[1,2-b]quinolin-10,13(9H,15H)-dione).

Reaction reagents and conditions that are commonly used for peptide synthesis can be employed for the reaction. There are various kinds of active ester. For example, it can be produced by reacting phenols such as p-nitrophenol, N-hydroxy benzotriazole, N-hydroxy succinimide, or the like, with the carboxylic acid (5) using a condensing agent such as N,N′-dicyclohexylcarbodiimide or 1-ethyl-3-(3-dimethylaminopropyl)carbodiimide hydrochloride. Further, the active ester can be also produced by a reaction of the carboxylic acid (5) with pentafluorophenyl trifluoroacetate or the like; a reaction of the carboxylic acid (5) with 1-benzotriazolyl oxytripyrrolidinophosphonium hexafluorophosphite; a reaction of the carboxylic acid (5) with diethyl cyanophosphonate (salting-in method); a reaction of the carboxylic acid (5) with triphenylphosphine and 2,2′-dipyridyl disulfide (Mukaiyama’s method); a reaction of the carboxylic acid (5) with a triazine derivative such as 4-(4,6-dimethoxy-1,3,5-triazin-2-yl)-4-methylmorpholinium chloride (DMTMM); or the like. Further, the reaction can be also performed by, e.g., an acid halide method by which the carboxylic acid (5) is treated with acid halide such as thionyl chloride and oxalyl chloride in the presence of a base.

By reacting the active ester, mixed acid anhydride, or acid halide of the carboxylic acid (5) obtained as above with the compound (4) in the presence of a suitable base in an inert solvent at a reaction temperature of -78° C. to 150° C., the compound (6) can be produced. Meanwhile, “inert solvent” indicates a solvent which does not inhibit a target reaction for which the solvent is used.

Specific examples of the base used for each step described above can include carbonate, alkoxide, hydroxide, or hydride of an alkali metal or an alkali earth metal including sodium carbonate, potassium carbonate, sodium ethoxide, potassium butoxide, sodium hydroxide, potassium hydroxide, sodium hydride, and potassium hydride, organometallic base represented by an alkyl lithium including n-butyl lithium, dialkylamino lithium including lithium diisopropylamide; organometallic base of bissilylamine including lithium bis(trimethylsilyl)amide; and organic base including tertiary amine or nitrogen-containing heterocyclic compound such as pyridine, 2,6-lutidine, collidine, 4-dimethylaminopyridine, triethylamine, N-methylmorpholine, diisopropylethylamine, and diazabicyclo[5.4.0]undec-7-ene (DBU).

Examples of the inert solvent which is used for the reaction of the present invention include a halogenated hydrocarbon solvent such as dichloromethane, chloroform, and carbon tetrachloride; an ether solvent such as tetrahydrofuran, 1,2-dimethoxyethane, and dioxane; an aromatic hydrocarbon solvent such as benzene and toluene; and an amide solvent such as N,N-dimethylformamide, N,N-dimethylacetamide, and N-methylpyrrolidin-2-one. In addition to them, a sulfoxide solvent such as dimethyl sulfoxide and sulfolane; a ketone solvent such as acetone and methyl ethyl ketone; and an alcohol solvent such as methanol and ethanol may be used in some case. Further, these solvents may be mixed for use.

As for the protecting group P¹ for the terminal amino group of the compound (6), a protecting group for an amino group which is generally used for peptide synthesis, for example, tert-butyloxy carbonyl group, 9-fluorenylmethyloxy carbonyl group, and benzyloxy carbonyl group, can be used. Examples of the other protecting group for an amino group can include an alkanoyl group such as acetyl group; an alkoxycarbonyl group such as methoxycarbonyl group and ethoxycarbonyl group; an arylmethoxy carbonyl group such as paramethoxybenzyloxy carbonyl group, and para (or ortho)nitroybenzyloxy carbonyl group; an arylmethyl group such as benzyl group and triphenyl methyl group; an aroyl group such as benzoyl group; and an aryl sulfonyl group such as 2,4-dinitrobenzene sulfonyl group and orthonitrobenzene sulfonyl group. The protecting group P¹ can be selected depending on, e.g., properties of a compound having an amino group to be protected.

By deprotecting the protecting group P¹ for the terminal amino group of the compound (6) obtained, the compound (7) can be produced. For this deprotection, reagents and conditions can be selected depending on the protecting group.

The compound (9) can be produced by derivatizing the peptide carboxylic acid (8) having the N terminal protected with P² into an active ester, mixed acid anhydride, or the like and reacting it with the compound (7) obtained. The reaction conditions, reagents, base, and inert solvent used for forming a peptide bond between the peptide carboxylic acid (8) and the compound (7) can be suitably selected and used from those described for the synthesis of the compound (6). The protecting group P² can be suitably selected and used from those described for the protecting group of the compound (6), and the selection can be made based on, e.g., the properties of the compound having an amino group to be protected. As it is generally used for peptide synthesis, by repeating sequentially the reaction and deprotection of the amino acid or peptide constituting the peptide carboxylic acid (8) for elongation, the compound (9) can be also produced.

By deprotecting the protecting group P² for the amino group of the compound (9) obtained, the compound (10) can be produced. For this deprotection, reagents and conditions can be selected depending on the protecting group.

It is possible to produce the compound (2) by derivatizing the carboxylic acid (11) into an active ester, mixed acid anhydride, acid halide, or the like and reacting it with the compound (10) obtained. The reaction conditions, reagents, base, and inert solvent used for forming a peptide bond between the carboxylic acid (11) and the compound (10) can be suitably selected and used from those described for the synthesis of the compound (6).

The compound (9) can be also produced by the following method, for example.

The compound (13) can be produced by derivatizing the peptide carboxylic acid (8) having the N terminal protected with P² into active ester, mixed acid anhydride, or the like and reacting it in the presence of a base with the amine compound (12) having the carboxy group protected with P³. The reaction conditions, reagents, base, and inert solvent used for forming a peptide bond between the peptide carboxylic acid (8) and the compound (12) can be suitably selected and used from those described for the synthesis of the compound (6).

The protecting group P² for the amino group of the compound (13) may be protected with a protecting group which is commonly used.

Specifically, examples of the protecting group for a hydroxyl group include an alkoxymethyl group such as methoxymethyl group; an arylmethyl group such as benzyl group, 4-methoxybenzyl group, and triphenylmethyl group; an alkanoyl group such as acetyl group; an aroyl group such as benzoyl group; and a silyl group such as tert-butyl diphenylsilyl group. Carboxy group can be protected, e.g., as an ester with an alkyl group such as methyl group, ethyl group, and tert-butyl group, an allyl group, or an arylmethyl group such as benzyl group. Examples of the protecting group for an amino group include, for example, an alkyloxy carbonyl group such as tert-butyloxy carbonyl group, methoxycarbonyl group, and ethoxycarbonyl group; allyloxycarbonyl group, or an arylmethoxy carbonyl group such as 9-fluorenylmethyloxy carbonyl group, benzyloxy carbonyl group, paramethoxybenzyloxy carbonyl group, and para (or ortho)nitroybenzyloxy carbonyl group; an alkanoyl group such as acetyl group; an arylmethyl group such as benzyl group and triphenyl methyl group; an aroyl group such as benzoyl group; and an aryl sulfonyl group such as 2,4-dinitrobenzene sulfonyl group or orthonitrobenzene sulfonyl group.

As for the protecting group P³ for a carboxy group, a protecting group commonly used as a protecting group for a carboxy group in organic synthetic chemistry, in particular, peptide synthesis can be used. Specific examples include esters with an alkyl group such as a methyl group, an ethyl group, or a tert-butyl, allyl esters, and benzyl esters, and the protective group can be suitably selected from the above-described protective groups. In such case, it is preferred that the protecting group for an amino group and the protecting group for a carboxy group can be those preferably removed by a different method or different conditions. For example, a representative example includes a combination in which P² is a tert-butyloxy carbonyl group and P³ is a benzyl group. The protecting groups can be selected from the aforementioned ones depending on, e.g., the properties of a compound having an amino group and a carboxy group to be protected. For removal of the protecting groups, reagents and conditions can be selected depending on the protecting group.

By deprotecting the protecting group P³ for the carboxy group of the compound (13) obtained, the compound (14) can be produced. For this deprotection, reagents and conditions are selected depending on the protecting group.

The compound (9) can be produced by derivatizing the compound (14) obtained into active ester, mixed acid anhydride, acid halide, or the like and reacting with the compound (4) in the presence of a base. For the reaction, reaction reagents and conditions that are generally used for peptide synthesis can be also used, and the reaction conditions, reagents, base, and inert solvent used for the reaction can be suitably selected from those described for the synthesis of the compound (6).

The compound (2) can be also produced by the following method, for example.

By deprotecting the protecting group P² for the amino group of the compound (13), the compound (15) can be produced. For this deprotection, reagents and conditions can be selected depending on the protecting group.

The compound (16) can be produced by derivatizing the carboxylic acid derivative (11) into active ester, mixed acid anhydride, acid halide, or the like and reacting it with the compound (15) obtained in the presence of a base. The reaction conditions, reagents, base, and inert solvent used for forming an amide bond between the peptide carboxylic acid (11) and the compound (15) can be suitably selected from those described for the synthesis of the compound (6).

By deprotecting the protecting group for the carboxy group of the compound (16) obtained, the compound (17) can be produced. This deprotection can be carried out similarly to the deprotection at carboxy group for producing the compound (14).

The compound (2) can be produced by derivatizing the compound (17) into active ester, mixed acid anhydride, acid halide, or the like and reacting it with the compound (4) in the presence of a base. For the reaction, reaction reagents and conditions that are generally used for peptide synthesis can be also used, and the reaction conditions, reagents, base, and inert solvent used for the reaction can be suitably selected from those described for the synthesis of the compound (6).

### Production Method 3

The compound represented by the formula (2) of an intermediate can be also produced by the following method.

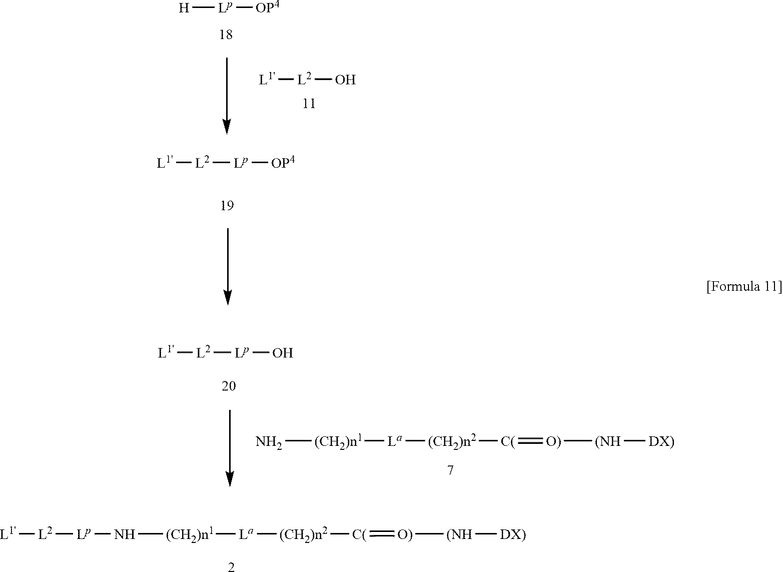

In the formula, L^{1'} corresponds to L¹ having a structure in which the terminal is converted to a maleimidyl group, and P⁴ represents a protecting group.

The compound (19) can be produced by derivatizing the compound (11) into active ester, mixed acid anhydride, or the like and reacting it in the presence of a base with the peptide carboxylic acid (18) having the C terminal protected with P⁴. The reaction conditions, reagents, base, and inert solvent used for forming a peptide bond between the peptide carboxylic acid (18) and the compound (11) can be suitably selected from those described for the synthesis of the compound (6). The protecting group P⁴ for the carboxy group of the compound (18) can be suitably selected from the protecting group described above.

By deprotecting the protecting group for the carboxy group of the compound (19) obtained, the compound (20) can be produced. This deprotection can be performed similar to the deprotection of the carboxy group for producing the compound (14).

The compound (2) can be produced by derivatizing the compound (20) obtained into active ester, mixed acid anhydride, or the like and reacting it with the compound (7). For the reaction, reaction reagents and conditions that are generally used for peptide synthesis can be also used, and the reaction conditions, reagents, base, and inert solvent used for the reaction can be suitably selected from those described for the synthesis of the compound (6).

### Production Method 4

Herein below, the method for producing the compound (10b) having n¹ = 1, L^{a} = O in the production intermediate (10) described in Production method 2 is described in detail. The compound represented by the formula (10b), a salt or a solvate thereof can be produced according to the following method, for example.

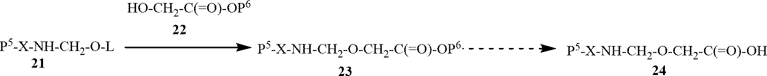

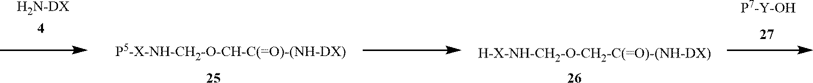

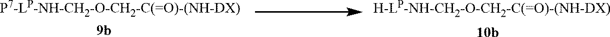

In the formula, L^{P} is as defined above, L represents an acyl group which is an alkanoyl group such as an acetyl group or an alloy group such as a benzoyl group, a hydrogen atom, or the like, X and Y each represent an oligopeptide consisting of 1 to 3 amino acids, P⁵ and P⁷ each represent a protecting group for an amino group, and P⁶ represents a protecting group for a carboxy group.

A compound represented by the formula (21) can be produced by using or applying the method described in Japanese Patent Laid-Open No. 2002-60351 or the literature (J. Org. Chem., Vol. 51, page 3196, 1986), and, by conducting removal of the protecting groups or modification of the functional groups, if necessary. Alternatively, it can be also obtained by treating an amino acid with a protected terminal amino group or acid amide of oligopeptide with protected amino group with aldehyde or ketone.

By reacting the compound (21) with the compound (22) having a hydroxyl group at a temperature ranging from under temperature conditions of cooling to room temperature in an inert solvent in the presence of an acid or a base, the compound (23) can be produced.

Examples of the acid which may be used here can include inorganic acid such as hydrofluoric acid, hydrogen chloride, sulfuric acid, nitric acid, phosphoric acid, and boric acid; an organic acid such as acetic acid, citric acid, paratoluene sulfonic acid, and methanesulfonic acid; and a Lewis acid such as tetrafluoroborate, zinc chloride, tin chloride, aluminum chloride, and iron chloride. Among them, sulfonic acids, particularly, paratoluene sulfonic acid is preferable. As for the base, any one of the aforementioned base can be suitably selected and used. Preferred examples thereof include an alkali metal alkoxide such as potassium tert-butoxide; an alkali metal hydroxide such as sodium hydroxide and potassium hydroxide; alkali metal hydride such as sodium hydride and potassium hydride; organometallic base represented by dialkylamino lithium such as lithium diisopropylamide; and organometallic base of bissilylamine such as lithium bis(trimethylsilyl)amide. Examples of the solvent to be used for the reaction include an ether solvent such as tetrahydrofuran and 1,4-dioxane; and an aromatic hydrocarbon solvent such as benzene and toluene. Those solvents can be prepared as a mixture with water. Further, the protecting group for an amino group as exemplified by P⁵ is not particularly limited if it is a group commonly used for protection of an amino group. Representative examples include the protecting groups for an amino group that are described in Production method 2. However, in the present reaction, there may be a case in which the protecting group for an amino group as exemplified by P⁵ is cleaved off. In such case, it is necessary to perform a reaction with a suitable reagent for protecting an amino group as it may be required to introduce the protecting group again.

The compound (24) can be produced by removing the protecting group P⁶ of the compound (23). Herein, the representative examples of the protecting group for a carboxy group as exemplified by P⁶ are described in Production method 2, and a suitable one can be selected from them. In the compound (23), it is desirable that the protecting group P⁵ for an amino group and the protecting group P⁶ for a carboxy group are the protecting groups that can be removed by a different method or different conditions. For example, a representative example includes a combination in which P⁵ is a 9-fluorenylmethyloxy carbonyl group and P⁶ is a benzyl group. The protecting groups can be selected depending on, e.g., the properties of a compound having an amino group and a carboxy group to be protected. For removal of the protecting groups, reagents and conditions are selected depending on the protecting group.

The compound (26) can be produced by derivatizing the carboxylic acid (24) into active ester, mixed acid anhydride, acid halide, or the like and reacting it with the compound (4) or a pharmacologically acceptable salt thereof to produce the compound (25) followed by removing the protecting group P⁵ of the compound (25) obtained. For the reaction between the compound (4) and the carboxylic acid (24) and the reaction for removing the protecting group P⁶, the same reagents and reaction conditions as those described for Production method 2 can be used.

The compound (10b) can be produced by reacting the compound (26) with an amino acid having protected terminal amino group or the oligopeptide (27) having protected amino group to produce the compound (9b) and removing the protecting group P⁷ of the compound (9b) obtained. The protecting group for an amino group as represented by P⁷ is not particularly limited if it is generally used for protection of an amino group. Representative examples thereof include the protecting groups for an amino group that are described in Production method 2. For removing the protecting group, reagents and conditions are selected depending on the protecting group. For the reaction between the compound (26) and the compound (27), reaction reagents and conditions that are commonly used for peptide synthesis can be employed. The compound (10b) produced by the aforementioned method can be derivatized into the compound (1) of the present invention according to the method described above.

The anti-TROP2 antibody-drug conjugate of the present invention, when it is left in air or recrystallized, for example, for purification, may absorb moisture to have adsorption water or turn into a hydrate, and such a compound and a salt containing water are also included in the present invention.

A compound labeled with various radioactive or non-radioactive isotopes is also included in the present invention. One or more atoms constituting the antibody-drug conjugate of the present invention may contain an atomic isotope at non-natural ratio. Examples of the atomic isotope include deuterium (²H), tritium (³H), iodine-125 (¹²⁵I), and carbon-14 (¹⁴C). Further, the compound of the present invention may be radioactive-labeled with a radioactive isotope such as tritium (³H), iodine-125 (¹²⁵I), carbon-14 (¹⁴C), copper-64 (⁶⁴Cu), zirconium-89 (⁸⁹Zr), iodine-124 (¹²⁴I), fluorine-18 (¹⁸F), indium-111 (¹¹¹I), carbon-11 (¹¹C) and iodine-131 (¹³¹I). The compound labeled with a radioactive isotope is useful as a therapeutic or prophylactic agent, a reagent for research such as an assay reagent and an agent for diagnosis such as an in vivo diagnostic imaging agent. Without being related to radioactivity, any isotope variant type of the antibody-drug conjugate of the present invention is within the scope of the present invention.

### Antibody-Drug Conjugates (ADC)

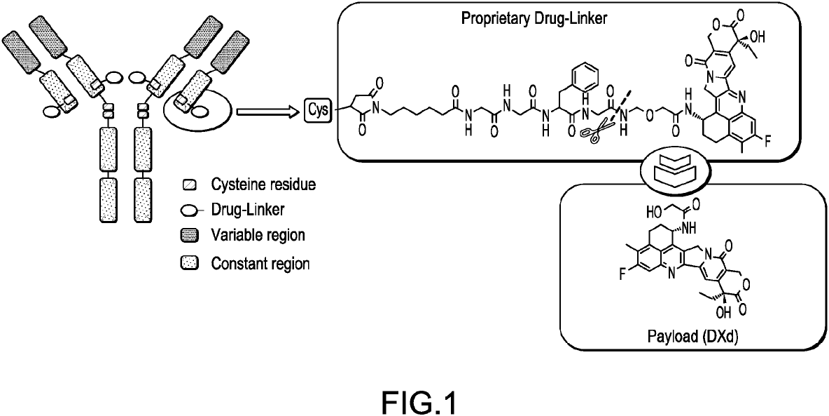

The present disclosure provides a TROP2-targeting antibody-drug conjugate (ADC) comprising an anti-TROP2 antibody and an anticancer compound, such as a topoisomerase I inhibitor (DXd). See FIG. 1. In some embodiments, the TROP2-targeting ADC may comprise Formula 13, as shown below:

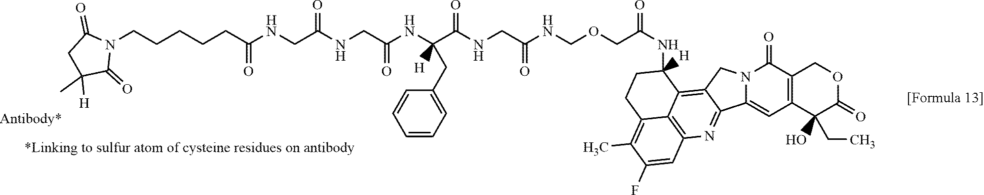

In some embodiments, the heavy chain of the ADC may comprise:

QVQLVQSGAEVKKPGASVKVSCKASGYTFTTAGMQWVRQAPGQGLEWMGW
INTHSGVPKYAEDFKGRVTISADTSTSTAYLQLSSLKSEDTAVYYCARSG
FGSSYWYFDVWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K (SEQ ID NO: 45).

In some embodiments, the light chain of the ADC may comprise:

DIQMTQSPSSLSASVGDRVTITCKASQDVSTAVAWYQQKPGKAPKLLIYS
ASYRYTGVPSRFSGSGSGTDFTLTISSLQPEDFAVYYCQQHYITPLTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO: 46).

The anti-TROP2 antibody-drug conjugate of the present invention exhibits a cytotoxic activity against cancer cells, and thus, it can be used as a drug, particularly as a therapeutic agent and/or prophylactic agent for cancer.

That is, the anti-TROP2 antibody-drug conjugate of the present invention can be selectively used as a drug for chemotherapy, which is a main method for treating cancer, and as a result, can delay development of cancer cells, inhibit growth thereof, and further kill the cancer cells. This can allow cancer patients to be free from symptoms caused by cancer or achieve improvement in QOL of cancer patients and attains a therapeutic effect by sustaining the lives of the cancer patients. Even if the anti-TROP2 antibody-drug conjugate of the present invention does not accomplish killing cancer cells, it can achieve higher QOL of cancer patients while achieving their longer-term survival, by inhibiting or controlling the growth of cancer cells.

In such drug therapy, it can be used as a drug alone as well as a drug in combination with an additional therapy in adjuvant therapy and can be combined with surgical operation, radiotherapy, hormone therapy, or the like. Furthermore, it can also be used as a drug for drug therapy in neoadjuvant therapy.

In addition to the therapeutic use as described above, an effect of suppressing the growth of minute metastatic cancer cells and further killing them by binding to these cancer cells can also be expected by virtue of the binding property of the antibody to the antigen. Particularly, when the expression of TROP2 is confirmed in primary cancer cells, inhibition of cancer metastasis or a prophylactic effect can be expected by administering the anti-TROP2 antibody-drug conjugate of the present invention. For example, an effect of inhibiting and killing cancer cells in a body fluid in the course of metastasis or an effect of, for example, inhibiting and killing minute cancer cells immediately after implantation in any tissue can be expected. Further, inhibition of cancer metastasis or a prophylactic effect can be expected, particularly, after surgical removal of cancer. Accordingly, an effect of inhibiting cancer metastasis can be expected.

The anti-TROP2 antibody-drug conjugate of the present invention can be expected to exert a therapeutic effect by administration as systemic therapy to patients, and additionally, by local administration to cancer tissues.

Examples of the cancer type to which the anti-TROP2 antibody-drug conjugate of the present invention is applied include lung cancer, kidney cancer, urothelial cancer, colorectal cancer, prostate cancer, glioblastoma multiforme, ovarian cancer, pancreatic cancer, breast cancer, melanoma, liver cancer, bladder cancer, gastric cancer, cervical cancer, head and neck cancer, or esophageal cancer, however, it is not limited to them as long as it is a cancer cell expressing, in a cancer cell as a treatment subject, a protein which the antibody within the antibody-drug conjugate can recognize.

The anti-TROP2 antibody-drug conjugate of the present invention can be preferably administered to a mammal, but it is more preferably administered to a human.

### Pharmaceutical Compositions and Modes of Administration

Substances used in a pharmaceutical composition containing an anti-TROP2 antibody-drug conjugate of the present invention can be suitably selected and applied from formulation additives or the like that are generally used in the art, in view of the dosage or administration concentration.

The anti-TROP2 antibody-drug conjugate of the present invention can be administered as a pharmaceutical composition containing at least one pharmaceutically suitable ingredient. For example, the pharmaceutical composition may typically contain at least one pharmaceutical carrier (for example, sterilized liquid). In some embodiments, the liquid includes, for example, water and oil (petroleum oil and oil of animal origin, plant origin, or synthetic origin). The oil may be, for example, peanut oil, soybean oil, mineral oil, or sesame oil. Water is a more typical carrier when the pharmaceutical composition is intravenously administered. Saline solution, an aqueous dextrose solution, and an aqueous glycerol solution can be also used as a liquid carrier, in particular, for an injection solution. A suitable pharmaceutical vehicle is known in the art. If desired, the composition above may also contain a trace amount of a moisturizing agent, an emulsifying agent, or a pH buffering agent. Examples of suitable pharmaceutical carrier are disclosed in “Remington’s Pharmaceutical Sciences” by E. W. Martin. The formulations correspond to an administration mode.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like.

Additionally, the disclosed pharmaceutical compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiment, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Various delivery systems are known and they can be used for administering the anti-TROP2 antibody-drug conjugate of the present invention. Examples of the administration route include intradermal, intramuscular, intraperitoneal, intravenous, and subcutaneous routes, but not limited thereto. The administration can be made by injection or bolus injection, for example. According to a specific preferred embodiment, the administration of the antibody-drug conjugate is performed by injection. Parenteral administration is a preferred administration route.

According to a representative embodiment, the pharmaceutical composition is prescribed, as a pharmaceutical composition suitable for intravenous administration to human, according to the conventional procedures. A composition for intravenous administration is typically a solution in a sterile and isotonic aqueous buffer solution. If necessary, the drug may contain a solubilizing agent and local anesthetics to alleviate pain at injection site (for example, lignocaine). Generally, the ingredient above is provided individually as any one of lyophilized powder or an anhydrous concentrate contained in a container which is obtained by sealing in an ampoule or a sachet having an amount of the active agent or as a mixture in a unit dosage form. When the drug is in the form of administration by injection, it may be administered from an injection bottle containing water or saline of sterile pharmaceutical grade. When the drug is administered by injection, an ampoule of sterile water or saline for injection may be provided such that the aforementioned ingredients are admixed with each other before administration.

The pharmaceutical composition of the present invention may be a pharmaceutical composition containing only the anti-TROP2 antibody-drug conjugate of the present application or a pharmaceutical composition containing the anti-TROP2 antibody-drug conjugate and at least one cancer treating agent other than the conjugate. The anti-TROP2 antibody-drug conjugate of the present invention can be administered with other cancer treating agent concurrently or in a series. The anti-cancer effect may be enhanced accordingly. Another anti-cancer agent used for such purpose may be administered to an individual simultaneously with, separately from, or subsequently to the antibody-drug conjugate, and it may be administered while varying the administration interval for each. Examples of the cancer treating agent include abraxane, paclitaxel, cisplatin, gemcitabine, irinotecan (CPT-11), paclitaxel, pemetrexed, sorafenib, vinorelbine, drugs described in International Publication No. WO 2003/038043, LH-RH analogues (leuprorelin, goserelin, or the like), estramustine phosphate, estrogen antagonist (tamoxifen, raloxifene, or the like), and an aromatase inhibitor (anastrozole, letrozole, exemestane, or the like), but it is not limited as long as it is a drug having an antitumor activity.

The pharmaceutical composition can be formulated into a lyophilization formulation or a liquid formulation as a formulation having desired composition and required purity. When formulated as a lyophilization formulation, it may be a formulation containing suitable formulation additives that are used in the art. Also for a liquid formulation, it can be formulated as a liquid formulation containing various formulation additives that are used in the art.

Composition and concentration of the pharmaceutical composition may vary depending on administration method. However, the anti-TROP2 antibody-drug conjugate contained in the pharmaceutical composition of the present invention can exhibit the pharmaceutical effect even at a small dosage when the antibody-drug conjugate has higher affinity for an antigen, that is, higher affinity (= lower Kd value) in terms of the dissociation constant (that is, Kd value) for the antigen. Thus, for determining dosage of the antibody-drug conjugate, the dosage can be determined in view of a situation relating to the affinity between the antibody-drug conjugate and antigen. When the antibody-drug conjugate of the present invention is administered to a human, for example, about 0.001 to 100 mg/kg can be administered once or administered several times with an interval of one time for 1 to 180 days.

### TROP2-Expressing Cancers

TROP2 is highly expressed in epithelial cancers, and its expression is associated with poor survival. Examples of TROP2-expressing cancers include, but are not limited to, lung cancer, kidney cancer, urothelial cancer, colorectal cancer, prostate cancer, glioblastoma multiforme, ovarian cancer, pancreatic cancer, breast cancer, melanoma, liver cancer, bladder cancer, gastric cancer, cervical cancer, head and neck cancer, and esophageal cancer. Any of these cancers may be treated by the disclosed ADC and ADC dosing regimens. However, it is to be understood that as long as a cancer cell expresses TROP2, it may be treated according to the disclosed methods even if it does not fall within the foregoing recited categories of cancer.

Non-small cell lung cancer (NSCLC) is a type of lung cancer that is particularly suitable for treatment utilizing the disclosed ADC and dosing regimens. For instance, Examples 5-7 detail Phase I clinical studies in which the disclosed ADC is administered to subjects with NSCLC.

The disclosed TROP2-targeting ADC can be used for treating any of the foregoing TROP2-expressing cancers.

### Methods of Treatment and Uses

The present disclosure provides methods of treating cancer comprising administering an anti-TROP2 antibody-drug conjugate as disclosed herein. Also provided herein are any of the disclosed anti-TROP ADC for use in treating a cancer.

In some embodiments, the cancer is a TROP2-expressing cancer. TROP2-expressing cancers may include, but are not limited to, lung cancer (e.g., non-small cell lung cancer or NSCLC), kidney cancer, urothelial cancer, colorectal cancer, prostate cancer, glioblastoma multiforme, ovarian cancer, pancreatic cancer, breast cancer, melanoma, liver cancer, bladder cancer, gastric cancer, cervical cancer, head and neck cancer, and esophageal cancer.

For the purposes of the present disclosure, the term “TROP2-overexpressing cancer” is not particularly limited as long as it is recognized as TROP2-overexpressing cancer by those skilled in the art. Preferred examples of the TROP2-overexpressing cancer can include cancer given a high score for the expression of TROP2 in an immunohistochemical method (IHC) or an in situ hybridization method (ISH). The in situ hybridization method of the present invention includes a fluorescence in situ hybridization method (FISH) and a dual color in situ hybridization method (DISH).

The method for scoring the degree of TROP2 expression by the immunohistochemical method, or the method for determining positivity or negativity to TROP2 expression by the in situ hybridization method is not particularly limited as long as it is recognized by those skilled in the art.

The ADC and the treatment methods and uses of the present invention can be preferably used for the treatment of inoperable or recurrent cancer.

In some embodiments, the ADC and the treatment methods and uses of the present invention can also be used as a pharmaceutical composition for treatment of cancer comprising the antibody-drug conjugate used in the present invention, a salt thereof, or a hydrate thereof as an active component, and a pharmaceutically acceptable formulation component.

In some embodiments, the ADC and the treatment methods and uses of the present invention exhibit excellent antitumor activity against cancer that exhibits resistance to an existing anticancer drug (i.e., resistant cancer), particularly, cancer that has acquired resistance to an existing anticancer drug (i.e., secondary resistant cancer). Thus, the ADC for treatment of the present invention exerts a remarkable antitumor effect when applied to a patient group with cancer having resistance to an existing anticancer drug (patients having a history of treatment with an existing anticancer drug) among cancer patients. In particular, the cancer being treated may be resistant to or refractory from treatment with an EGFR-inhibitor treatment (i.e., gefitinib, erlotinib, osimertinib, affatinib), an ALK-inhibitor treatment (i.e., alectinib, crizotinib, ceritinib), a platinum-based chemotherapeutics (i.e., cisplatin, carboplatin), and/or a checkpoint inhibitor treatment (i.e., nivolumab, pembrolizumab, atezolizumab, avelumab, ipilimumab, durvalumab, tislelizumab, sintilimab, cemiplimab).

The ADC for treatment of the present invention can administered instead of existing anticancer drugs or in combination with these existing anticancer drugs to a cancer patient to thereby exhibit a high therapeutic effect on, for example, cancer that has acquired resistance to these existing anticancer drugs.

Thus, in some embodiments of the disclosed methods and use, the cancer being treated may be a resistant form of lung cancer (e.g., non-small cell lung cancer or NSCLC), kidney cancer, urothelial cancer, colorectal cancer, prostate cancer, glioblastoma multiforme, ovarian cancer, pancreatic cancer, breast cancer, melanoma, liver cancer, bladder cancer, gastric cancer, cervical cancer, head and neck cancer, and esophageal cancer.

The ADC and methods or uses for treatment of the present invention can delay development of cancer cells, inhibit growth thereof, and further kill cancer cells. These effects can allow cancer patients to be free from symptoms caused by cancer or achieve improvement in quality of life (QOL) of cancer patients and attain a therapeutic effect by sustaining the lives of the cancer patients. Even if the anti-TROP2 antibody-drug conjugate of the present invention does not accomplish killing cancer cells, it can provide higher QOL of cancer patients while achieving longer-term survival, by inhibiting or controlling the growth of cancer cells.

In some embodiments of the disclosed methods and uses, the ADC can be used as a drug alone, or it can be used as a drug in combination with an additional therapy in adjuvant therapy and can be combined with surgical operation, radiotherapy, hormone therapy, or the like. Furthermore, it can also be used as a drug for drug therapy in neoadjuvant therapy. In some embodiments, the ADC may be combined with, for example, an anticancer agent including, but not limited to, abraxane, paclitaxel, cisplatin, carboplatin, gemcitabine, irinotecan (CPT-11), pemetrexed, sorafenib, vinorelbine, drugs described in International Publication No. WO 2003/038043, LH-RH analogues (leuprorelin, goserelin, or the like), estramustine phosphate, estrogen antagonist (tamoxifen, raloxifene, or the like), an aromatase inhibitor (anastrozole, letrozole, exemestane, or the like), an EGFR-inhibitor treatment (gefitinib, erlotinib, osimertinib, affatinib), an ALK-inhibitor treatment (alectinib, crizotinib, ceritinib), and/or a checkpoint inhibitor treatment (nivolumab, pembrolizumab, atezolizumab, avelumab, ipilimumab, durvalumab, tislelizumab, sintilimab, cemiplimab).

In addition to the therapeutic methods and uses described above, a prophylactic effect of suppressing the growth of small metastatic cancer cells and further killing them can also be expected. Particularly, when the expression of TROP2 is confirmed in primary cancer cells, inhibition of cancer metastasis or a prophylactic effect can be expected by administering the anti-TROP2 antibody-drug conjugate of the present invention. For example, an effect of inhibiting and killing cancer cells in a body fluid in the course of metastasis or an effect of, for example, inhibiting and killing small cancer cells immediately after implantation in any tissue can be expected. Accordingly, inhibition of cancer metastasis or a prophylactic effect can be expected, particularly, after surgical removal of cancer.

In some embodiments of the methods and uses, a subject with cancer (e.g., a TROP2-expressing cancer) may be administered about 0.1 to about 15 mg/kg, about 0.5 to about 12 mg/kg, about 1.0 to about 10 mg/kg, or about 4 to about 8 mg/kg. In other words, in some embodiments the dose of the ADC administered to the subject may be about 0.1, about 0.2, about 0.3, about 0.4, about 0.5, about 0.6, about 0.7, about 0.8, about 0.9, about 1.0, about 1.25, about 1.5, about 1.75, about 2.0, about 2.25, about 2.5, about 2.75, about 3.0, about 3.25, about 3.5, about 3.75, about 4.0, about 4.25, about 4.5, about 4.75, about 5.0, about 5.25, about 5.5, about 5.75, about 6.0, about 6.25, about 6.5, about 6.75, about 7.0, about 7.25, about 7.5, about 7.75, about 8.0, about 8.25, about 8.5, about 8.75, about 9.0, about 9.25, about 9.5, about 9.75, about 10.0, about 10.25, about 10.5, about 10.75, about 11.0, about 11.25, about 11.5, about 11.75, or about 12 mg/kg or more. In some embodiments, the dose of the ADC administered to the subject may be 0.1, 0.2, 0.3, 0.4, 0.5, 0.6, 0.7, 0.8, 0.9, 1.0, 1.25, 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25, 11.5, 11.75, or 12 mg/kg or more. In some embodiments, the dose may be about 2 mg/kg to about 10 mg/kg, about 2 mg/kg to about 8 mg/kg, about 4 mg/kg to about 10 mg/kg, about 4 mg/kg to about 8 mg/kg, about 6 mg/kg to about 10 mg/kg, or about 6 mg/kg to about 8 mg/kg. In preferred embodiments, the dose may be 2 mg/kg, 3 mg/kg, 4 mg/kg, 5 mg/kg, 6 mg/kg, 7 mg/kg, 8 mg/kg, 9 mg/kg, or 10 mg/kg, but more preferably 4 mg/kg, 6 mg/kg or 8 mg/kg.

In some embodiments of the methods and uses, the anti-TROP2 ADC or a pharmaceutical composition thereof is administered to a subject with cancer via parenteral administration. Preferred parenteral routes of administering include, but are not limited to, injections, such as intravenous, intramuscular, and subcutaneous injections. The anti-TROP2 antibody-drug conjugate used in the present invention can be expected to exert a therapeutic effect by application as systemic therapy to patients, and additionally, by local application to cancer tissues.

The timing or regimen of administration may be once every 1 week (q1w), once every 2 weeks (q2w), once every 3 weeks (q3w), once every 4 weeks (q4w), once every 5 weeks (q5w), once every 6 weeks (q6w), once every 7 weeks (q7w), once every 8 week (q8w), once every 9 weeks (q9w), or once every 10 weeks (q10w), but is preferably once every 3 or 4 weeks.

Dosage regimens may be adjusted to provide the optimum desired response (e.g., a therapeutic response like tumor regression or remission). For example, in some embodiments, dosage regimen may be 2 mg/kg once every 3 weeks (q3w), 4 mg/kg once every 3 weeks (q3w), 6 mg/kg once every 3 weeks (q3w), 8 mg/kg once every 3 weeks (q3w), 2 mg/kg once every 4 weeks (q4w), 4 mg/kg once every 4 weeks (q4w), 6 mg/kg once every 4 weeks (q4w), or 8 mg/kg once every 4 weeks (q4w). And in some embodiments, a single bolus may be administered, while in some embodiments, several divided doses may be administered over time or the dose may be proportionally reduced or increased as indicated by the situation.

Furthermore, while the subject of the methods and uses is generally a cancer patient, the age of the patient is not limited. The disclosed methods and uses are useful for treating cancer, malignant disease, or cancer cell proliferation with various recurrence and prognostic outcomes across all age groups and cohorts. Thus, in some embodiments, the subject may be a pediatric subject, while in other embodiments, the subject may be an adult subject.

The following examples are given to illustrate the present invention. It should be understood, however, that the invention is not to be limited to the specific conditions or details described in these examples.

### EXAMPLES

### Example 1 - Production of Antibody Drug Conjugates

According to production methods disclosed in International Publication No. WO2015/098099 and International Publication No. WO2017/002776, an anti-TROP2 antibody (e.g., an antibody comprising a heavy chain consisting of the amino acid sequence at amino acid positions 1 to 451 in SEQ ID NO: 45 and a light chain consisting of the amino acid sequence at amino acid positions 1 to 214 in SEQ ID NO: 46) was used to produce an antibody-drug conjugate (1) and an antibody-drug conjugate (2)(hereinafter referred to as the “antibody-drug conjugate (1)” and the “antibody-drug conjugate (2)”) in which the anti-TROP2 antibody is bound, via a thioether bond, to a drug linker represented by the following formula:

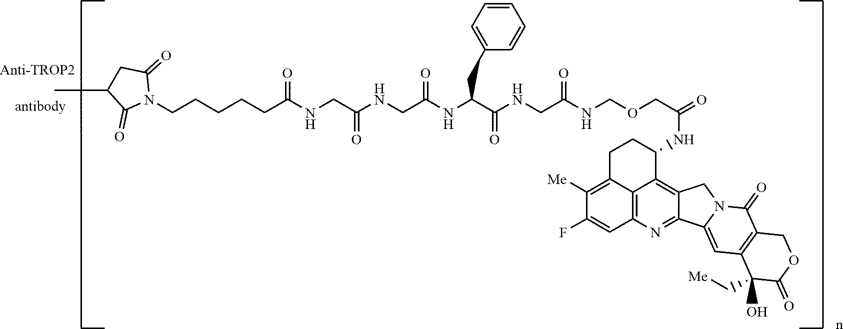

where n represents an average drug-to-antibody ratio (DAR) per single antibody molecule; and the value of n of the antibody-drug conjugate (1) falls within the range of 3.5 to 4.5; whereas, the value of n of the antibody-drug conjugate (2) falls within the range of 6.5 to 8.0.

The general structures and sequences of antibody-drug conjugate (1) and antibody-drug conjugate (2) are found in FIGS. 1 and 2.

### Example 2 - Test for Antitumor Effect of Antibody Drug Conjugates

For the purposes of this experiment, 5-6 weeks old, female, BALB/c nude mice (Charles River Laboratories Japan), were subjected to experiments. Human pancreatic adenocarcinoma cell line, CFPAC-1 cells, purchased from ATCC were suspended in saline, and 4 × 10⁶ cells were transplanted to a right-side portion of the body of each of the female nude mice. Fourteen days after transplantation, the mice were grouped (Day 0). In single administration groups (once in every three weeks), antibody-drug conjugates (1) and (2) were administered at a dose of 0.3 mg/kg or 1 mg/kg on Day 0. In frequent administration groups (once per week for three weeks), antibody-drug conjugates (1) and (2) were administered at a dose of 0.3 mg/kg on Day 0, Day 8 and Day 14. A vehicle administration group was determined as a control group. The tumor growth inhibition (TGI) on Day 22 was obtained by calculation. In none of the administration groups, particularly notable findings such as weight loss were confirmed.

Measurement/computation expression: the major axis and minor axis of tumors were measured by an electronic digital caliper (CD-15CX, Mitutoyo Corp.) twice per week and tumor volumes (mm³) were calculated. The computation expression is as shown below:

$$\begin{matrix}\text{Tumor volume} \left({\text{mm}}^{\text{3}}\right) \\ \text{= ½} \times \text{major axis} \left(\text{mm}\right) \times {\left[\text{minor axis} \left(\text{mm}\right)\right]}^{\text{2}}\end{matrix}$$

Tumor growth inhibition (TGI) was calculated according to the following computation expression:

$$\text{Tumor growth inhibition} \left(\text{\%}\right) \text{= 100} \times \left(\text{1-T/C}\right)$$

where T represents the average tumor volume of a test-substance administered mouse group; and C represents the average tumor volume of a control mouse group.

The antibody-drug conjugates (1) and (2) were each diluted with acetate buffered saline (pH5.5) (manufactured by Nacalai Tesque Inc., hereinafter referred to as “ABS buffer”). The dilution solution (10 mL/kg) was administered through the tail vein.

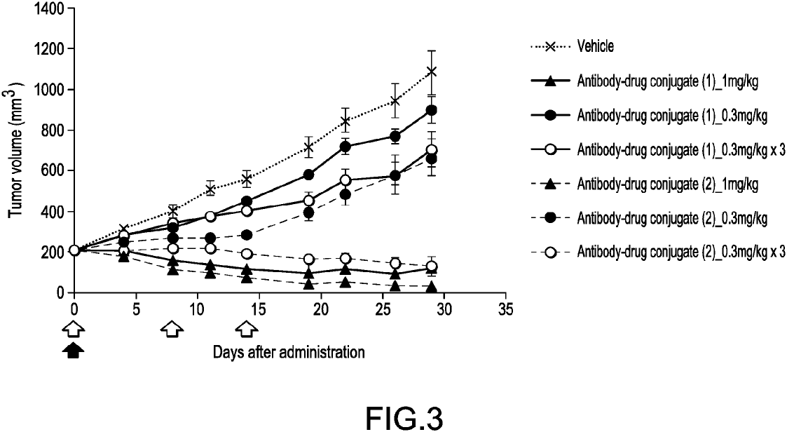

The antitumor effects of antibody-drug conjugates (1) and (2) are shown in FIG. 3. In the antibody-drug conjugate (1), TGI of a single administration group at a dose of 0.3 mg/kg was 15% and TGI of a single administration group at a dose of 1 mg/kg was 86%; whereas, TGI of the frequent administration group at a dose (0.3 mg/kg) was 34%. In the antibody-drug conjugate (2), TGI of a single administration group at a dose of 0.3 mg/kg was 43% and TGI of a single administration group at a dose of 1 mg/kg was 94%; whereas, TGI of the frequent administration group at a dose (0.3 mg/kg) was 80%.

From the above results, in both cases of the antibody-drug conjugates (1) and (2), when the single administration group at a dose of 1 mg/kg was compared to frequent administration group at a dose of a 0.3 mg/kg, both providing an almost equivalent dose in total, the TGI of the single administration group was higher than that of the frequent administration group. From this, it was demonstrated that a single administration method of administering the total dose for three weeks just once has more excellent efficacy than the frequent administration method of repeating administration of the dose for a week, three times. In comparison between the antibody-drug conjugates (1) and (2), the TGI of the single administration group of the antibody-drug conjugate (1) at a dose of 1 mg/kg is higher than the TGI of the single administration group of the antibody-drug conjugate (2) at a dose of 0.3 mg/kg and lower than the TGI of the single administration group of the antibody-drug conjugate (2) at a dose of 1 mg/kg. From this, it was demonstrated that the difference in therapeutic dose between antibody-drug conjugates (1) and (2) falls within the range of three fold.

### Example 3 - Safety Evaluation of Antibody Drug Conjugates

The antibody-drug conjugates (1) and (2) produced according to Example 1 were separately administered to a cross-reactive species, cynomolgus monkeys. More specifically, the antibody-drug conjugate (1) was administered at intervals of once in every three weeks, three times in total; whereas, the antibody-drug conjugate (2) was administered at intervals of once a week, twice in total. In the case of the antibody-drug conjugate (1), observation was continued up to the following day of the final administration. In the case of the antibody-drug conjugate (2), observation was continued up to the following week of the final administration. As a result, the highest non-severely toxic dose (HNSTD) of the antibody-drug conjugate (2) was less than 10 mg/kg; whereas, the HNSTD of the antibody-drug conjugate (1) was 30 mg/kg. Thus, it was demonstrated that the antibody-drug conjugate (1) has better safety than the antibody-drug conjugate (2).

### Example 4 - Estimation of Effective Dosage/Dose of Antibody-Drug Conjugate (1) in Humans

Hereinafter, “antibody-drug conjugate (1)” may also be referred to as “DS-1062a.”

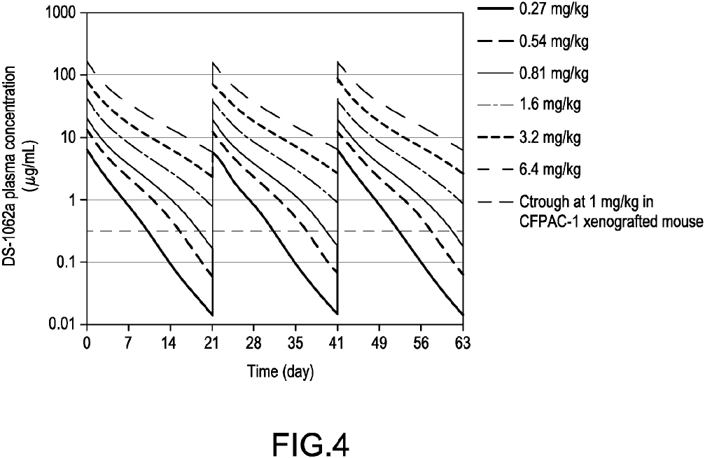

The antibody-drug conjugate (1) (i.e., DS-1062a)was intravenously administered once in a dose of 0.2, 0.6, 2 or 6 mg/kg to cynomolgus monkeys. Thereafter, based on the plasma concentration of the antibody-drug conjugate (1), pharmacokinetic parameters were calculated using a target mediated drug disposition model. Further, the plasma concentration change of antibody-drug conjugate with time during the repeated administration to human was estimated. The antibody-drug conjugate (1) was administered by repeating a dosing cycle consisting of 0.27, 0.54, 0.81, 1.6, 3.2, and 6.4 mg/kg once in every three weeks, three times (q3w × 3). The results are shown in FIG. 4. The plasma concentration change with time of the antibody-drug conjugate (1) estimated in humans was compared to the plasma concentration on Day 21 in a CFPAC-1 tumor-bearing mouse model. As a result, the doses, at which the plasma concentration estimated at the time of administration to humans once in every three weeks (q3w) exceeds the minimum concentration (1 mg/kg administration group, 0.312 µg/mL) showing a tumor regression effect in mice, in the half and most part of the administration interval, were 0.27 and 0.81 mg/kg, respectively. From this, it was estimated that the effective dosage/dose of the antibody-drug conjugate (1) in humans is 0.27 mg/kg or more, once in every three weeks.

### Example 5 - Initial Phase 1 Clinical Study

### Introduction

DS-1062a is a trophoblast cell-surface antigen 2 (TROP2)-targeting antibody-drug conjugate with a novel topoisomerase I inhibitor (Exatecan derivative; DXd). DS-1062a binds to TROP2 on the cell surface, internalizes and releases DXd into the cytoplasm after enzymatic processing which inhibits topoisomerase I and leads to apoptosis of the target cells.

TROP2 is highly expressed in epithelial cancers, including lung cancer, and is associated with poor survival. In preclinical studies, DS-1062a showed promising antitumor activity in xenograft mouse models.

### Purpose

The purpose of this study was to evaluate the safety and tolerability of DS-1062a and determine the maximum tolerated dose (MTD) and recommended dose for expansion (RDE) (see clinicaltrials.gov identifier NCT03401385).

### Study Design and Methods

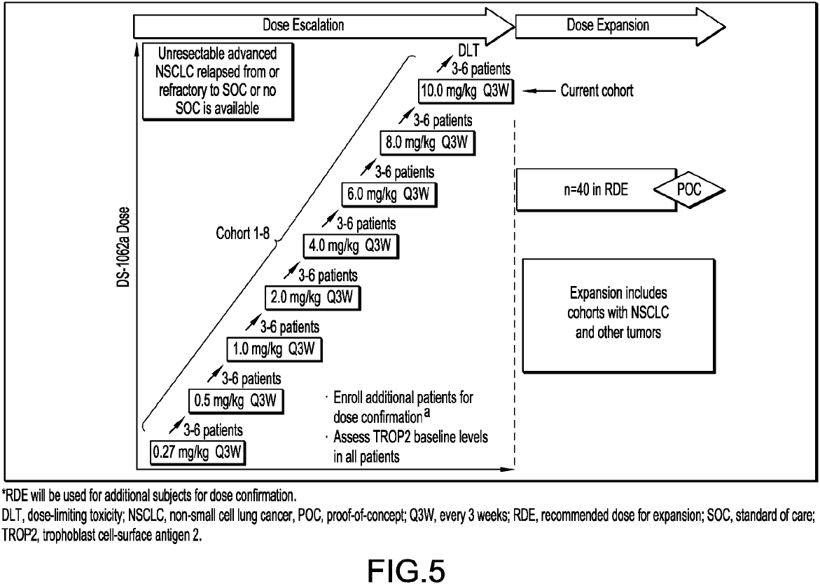

The Phase 1 study was a multicenter, open-label, multiple-dose, first-in-human study of DS-1062a, which enrolled subjects in the United States and Japan. The study included both a dose escalation arm and a dose expansion arm, as shown in FIG. 5. The dose escalation arm included a single intravenous infusion of DS-1062a and a 21-day dose-limiting toxicity (DLT) observation period (Cycle 1). The dose-expansion arm included administering to NSCLC subjects a dose of DS-1062a at the RDE.

The primary objection of the dose escalation arm was to identify the MTD for RDE and assess the safety and tolerability of the doses.

The primary objective for the dose expansion arm was to confirm the safety and tolerability of DS-1062a at the RDE.

Secondary objectives included measuring pharmacokinetic (PK) properties of DS-1062a, total TROP2 antibody, drug components, and antitumor activity of DS1062a. Exploratory objectives included evaluating biomarkers that correlated with a response to DS-1062a.

Inclusion criteria included: patients aged ≥20 years (Japan) or ≥18 years (United States) with pathologically documented, metastatic NSCLC without standard treatment option; Eastern Cooperative Oncology Group performance status 0 or 1; measurable disease based on RECIST version 1.1; a life expectancy of ≥3 months; and available tumor tissue for the measurement of recent TROP2 levels by immunohistochemistry.

Exclusion criteria included: patients with multiple primary malignancies (except adequately resected non-melanoma skin cancer, curatively treated in situ disease, or other solid tumors curatively treated with no evidence of disease for ≥3 years); or clinically significant/suspected lung disease.

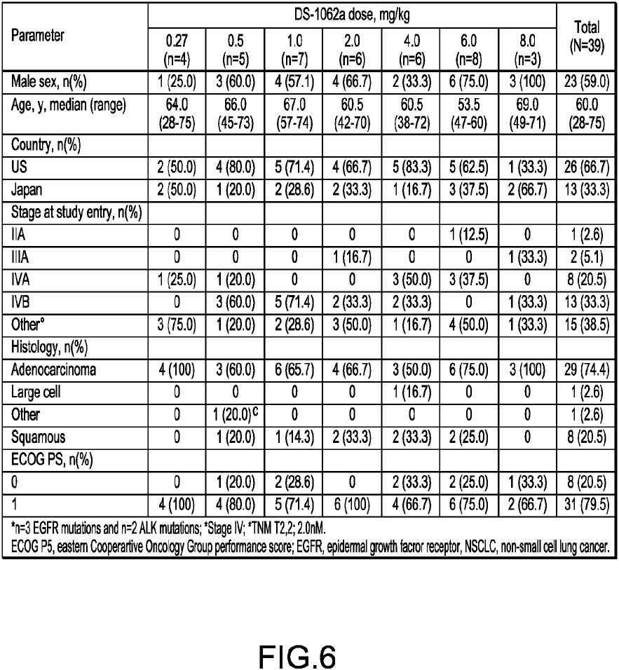

Patient assessments included echocardiogram or multigated acquisition scan, 12-lead electrocardiogram, AEs, PK, human anti-human antibodies, biomarkers, and tumor assessments at prespecified visits. The demographics and baseline characteristics of the patients enrolled in this initial Phase 1 study are shown in FIG. 6.

Dose escalation of DS-1062a to determine the MTD was guided by the modified continuous reassessment method using a Bayesian logistic regression model following escalation with the overdose control principle. The objective response rate (ORR) was summarized with 95% confidence intervals (Cl) using the Clopper-Pearson method; progression-free survival (PFS)/overall survival (OS) was summarized using the Kaplan-Meier method. Safety endpoints, PK parameters of DS-1062a, anti-TROP2 antibody, DXd, and plasma antidrug antibodies were summarized using descriptive statistics.

### Results

Thirty-nine patients were enrolled at cut-off among seven DS-1062a dosing groups, and the patient demographics and baseline characteristics. The patients (N=39) were exposed to a median (range) of 3.0 (1-10) treatment cycles with DS-1062a, over a median (range) duration of 8.86 (3.0-31.1) weeks.

Two patients required DS-1062a dose interruption (one in the 4 mg/kg group and one in the 8 mg/kg group), and 1 patient in the 6.0-mg/kg group required dose reduction. Overall, 23 (54.8%) patients discontinued from treatment with DS-1062a. The primary reason for discontinuation was PD per RECIST in 13 patients (n=4 each [0.5 and 2.0 mg/kg]; n=3 [1.0 mg/kg]; n=1 each [0.27 and 4.0 mg/kg]. Two patients discontinued due to clinical progression (1 each in the 0.27 mg/kg and 6.0 mg/kg), two patients withdrew (1 each in the 0.5 mg/kg and 4.0 mg/kg), and one patient discontinued based on a physician decision (1.0 mg/kg group). Five patients (n=3 in the 1.0-mg/kg and n=2 in the 0.27-mg/kg groups) discontinued due to “other” reasons.

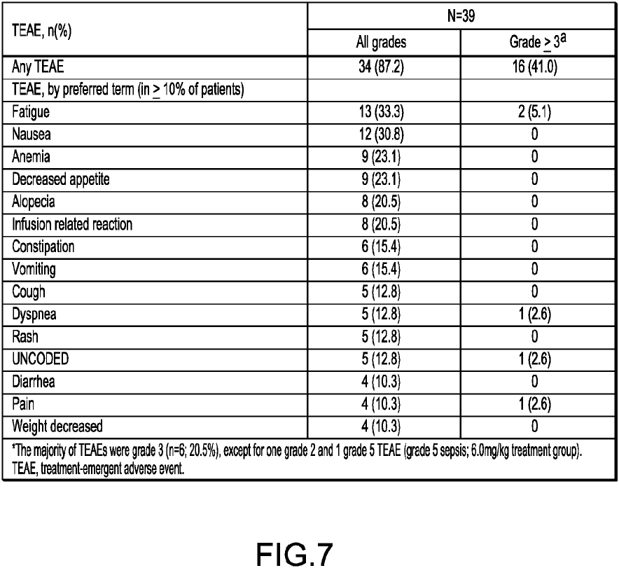

Overall, 87.2% (34/39) of patients reported ≥1 TEAE, but all except one of the reported TEAE were considered grade ≤3 (FIG. 7). The most frequent TEAE was fatigue, reported in 13 (33.3%) of patients. All grade 3 TEAEs were reported in only 1 patient each, except grade 3 fatigue, which was reported in 2 patients (1 each in the 0.5- and 2.0-mg/kg dosing groups).

Drug-related TEAEs were reported in 23/39 (59.0%) patients, with 21/23 (91.3%) of these of patients having these TEAEs as grade 1 or 2 in severity. The most frequent TEAEs (in ≥3 patients) by descending order of frequency were nausea (n=10); infusion-related reactions (n=8); fatigue (n=7); alopecia (n=6); vomiting (n=5); anemia and rash (n=4 each}; and decreased appetite and stomatitis (n=3 each). Infusion-related reactions were all were grade 1 or 2 events and were manageable/reversible.

Serious TEAEs were reported in 10/39 (25.6%) patients; the majority (n=8) were grade 3, and 1 each was grade 2 and grade 5 (grade 5 sepsis; 6.0 mg/kg dosing group). No serious TEAE occurred in more than 1 patient. Only 1 serious TEAE was considered drug-related (pyrexia, grade 2; 4.0-mg/kg dosing group).

One dose limiting toxicity (DLT) (maculopapular rash, grade 3; resolved) occurred in a patient in the 6.0-mg/kg dosing group; the MTD has not been reached.

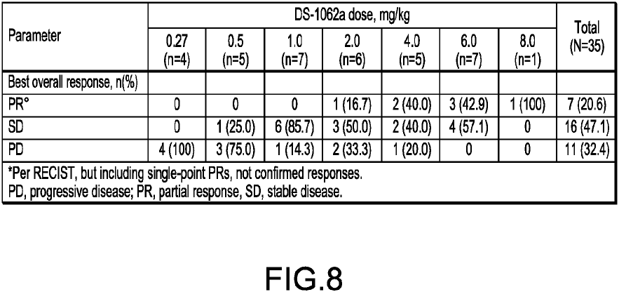

Of the 35 tumor-evaluable patients, 7 PRs (based on RECIST, but including single-point PRs, not yet confirmed responses) were observed, as shown in FIG. 8. Following the datacut, 3 additional PRs (all in the 8.0-mg dosing group), for a total of 10 PRs, were observed.

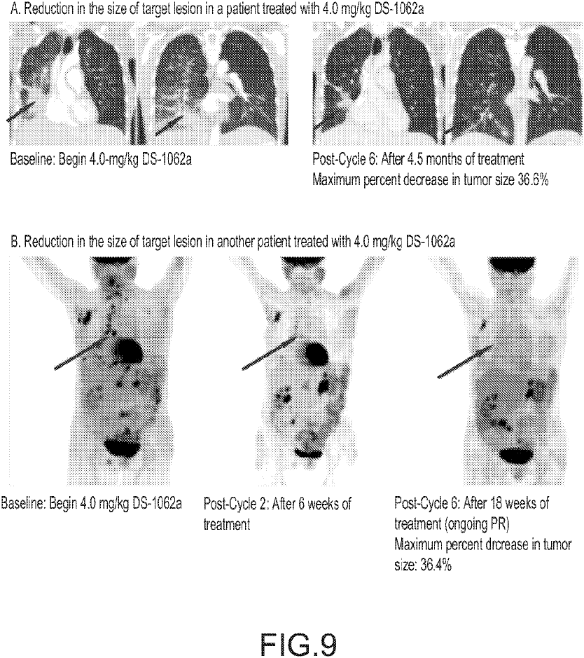

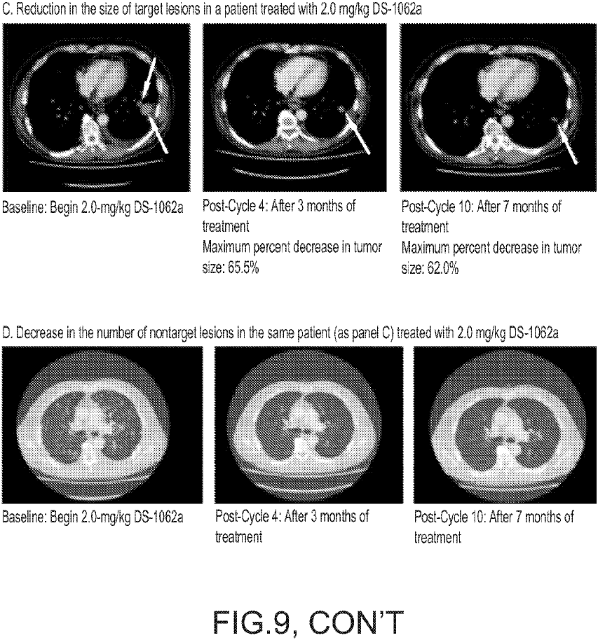

Computed (FIGS. 9 A, C, and D) and positron emission (FIG. 9B) were taken in three patients. Two patients in the 4.0-mg/kg dosing group demonstrated a maximum 36.6% (FIG. 9A) and 38.4% (FIG. 9B) decrease in tumor size 4.5 months following initiation of treatment with DS-1062a. Another patient in the 2-mg/kg dosing group demonstrated a maximum 65.5% decrease in tumor size 3 months following initiation of treatment with DS-1062a (FIG. 9C) and a notable decrease in the number of multiple lung metastases (non-target lesions) at 3- and 7-months post treatment initiation (FIG. 9D).

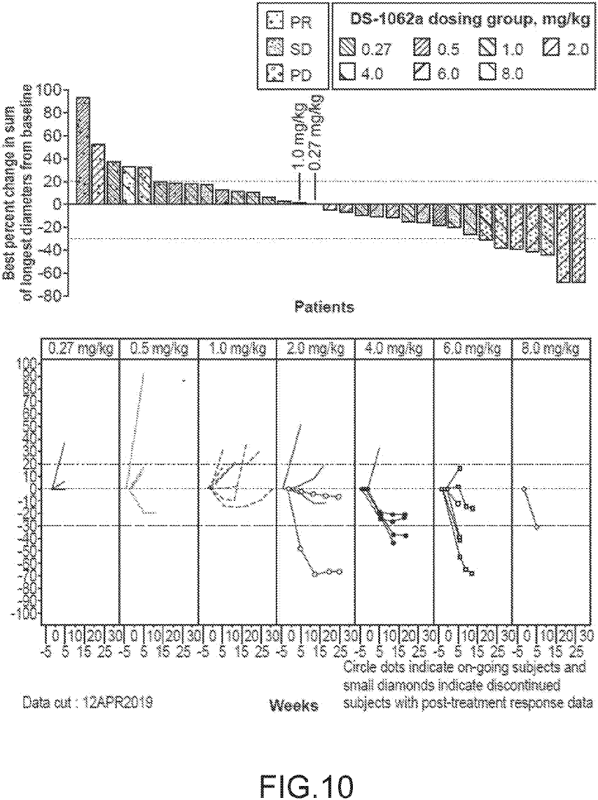

The best percent change in sum of longest dimensions from baseline in target lesion is illustrated in FIG. 10. The best percent change (68% tumor reduction) was observed in a patient in the 2.0-mg/kg dosing group.

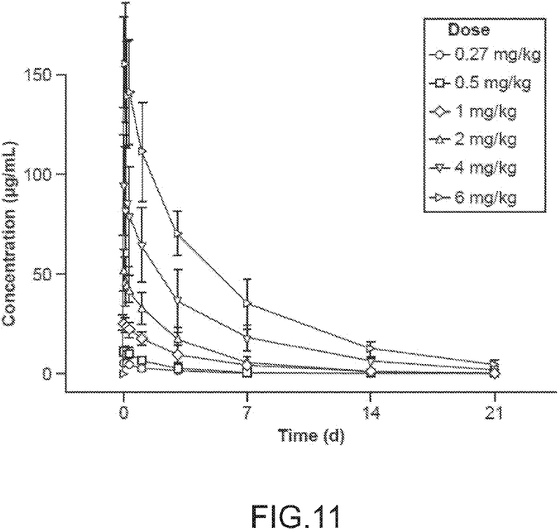

With respect to pharmacokinetics, systemic exposure to DS-1062a increased in an approximately dose-proportional manner, as shown in FIG. 11. Plasma levels of DS-1062a and total anti-TROP2 antibody were similar, suggesting DS-1062a was stable in circulation. Exposure of DXd was lower than that of DS-1062a.

### Summary

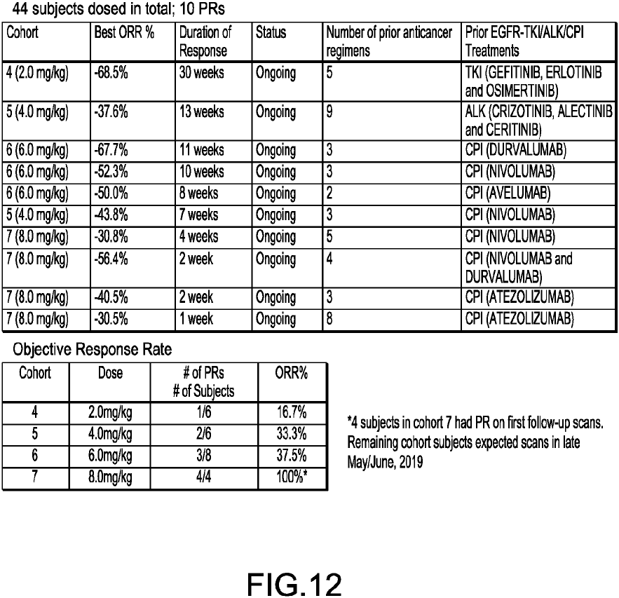

As of the datacut, DS-1062a was well tolerated. One DLT of grade 3 skin rash, which was transient and reversible, was observed in the 6.0-mg/kg dosing group. Ten PRs and 16 stable disease were observed with DS-1062a. Two of the patients with PRs had prior EGFR- or ALK-inhibitor treatment (i.e., alectinib, crizotinib, ceritinib, osimertinib). The overall efficacy rates of the study are provided in FIG. 12.

### Example 6 - Phase 1 Clinical Study as of New Cut-Off Date

After the initial datacut, additional patients were enrolled in the Phase 1 study, bringing the overall number of participants to fifty-nine (N=59). All patients had unresected advanced NSCLC tumors that were relapsed/refractory to standard of care (SOC). The patients were 57.7% male, 88.5% has Stage IV disease, 73.1% had adenocarcinoma histology, 80.8% had Eastern Cooperative Oncology Group performance status (ECOG PS) of 1, and 86.5% had failed prior immune checkpoint inhibitor treatment. The same dose escalation and dose expansion study design was used.

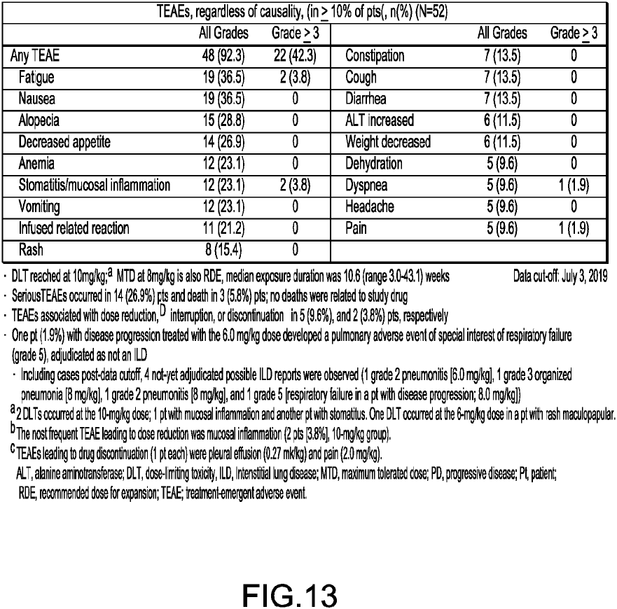

The number of patients in the Phase 1 study as of the new cut-off date with treatment-emergent adverse events (TEAEs), regardless of causality, is shown in FIG. 13. Briefly, dose limiting toxicity (DLT) reached at 10 mg/kg, and the maximum tolerated dose (MTD) was established at 8 mg/kg, which was also the recommended dose for expansion (RDE) in future dose expansion part. Median exposure duration for the patients was 10.6 (range 3.0 - 43.1) weeks. Serious TEAEs occurred in 14 (26.9%) patients and death in 3 (5.8%) patients; no deaths were related to the study drug. TEAEs associated with dose reduction, interruption, or discontinuation occurred in 5 (9.6%), 5 (9.6%), and 2 (3.8%) patients, respectively. One patient (1.9%) with disease progression treated with the 6.0 mg/kg dose developed a pulmonary adverse event of special interest of respiratory failure (grade 5), adjudicated as not an interstitial lung disease (ILD). Including cases post-data cut-off, 4 not-yet adjudicated possible ILD reports were observed (1 grade 2 pneumonitis [6.0 mg/kg], 1 grade 2 organized pneumonia [8 mg/kg], 1 grade 2 pneumonitis [8 mg/kg], and 1 grade 5 [respiratory failure in a patient with disease progression; 8.0 mg/kg]).

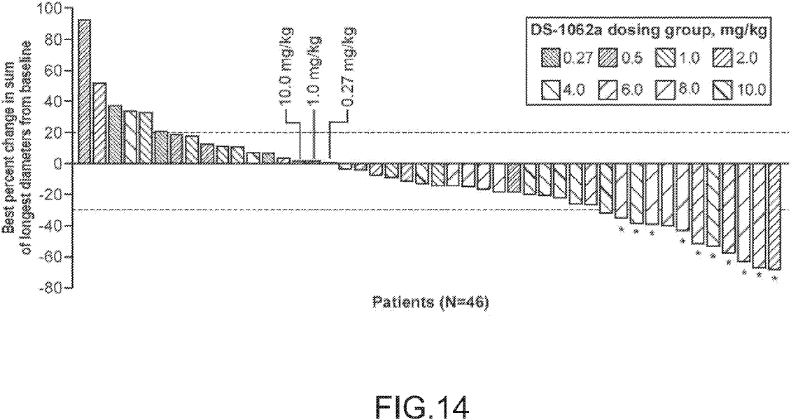

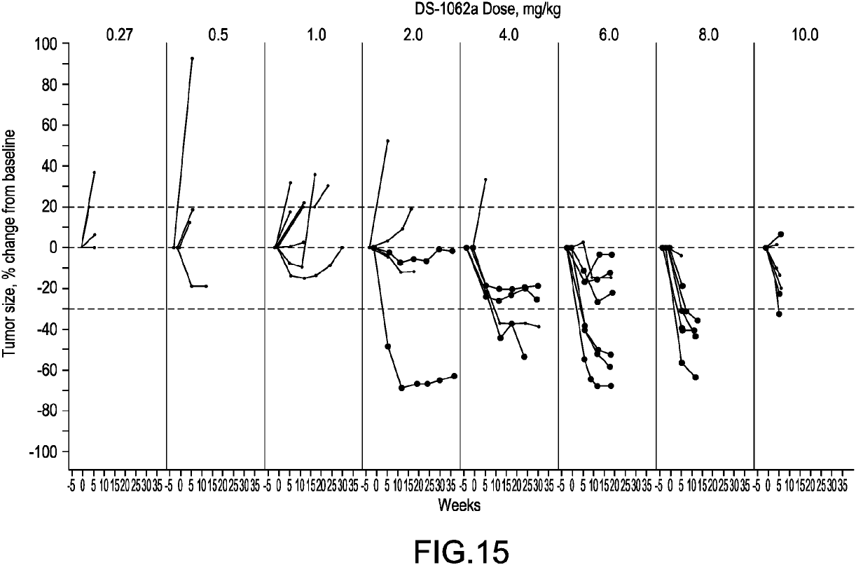

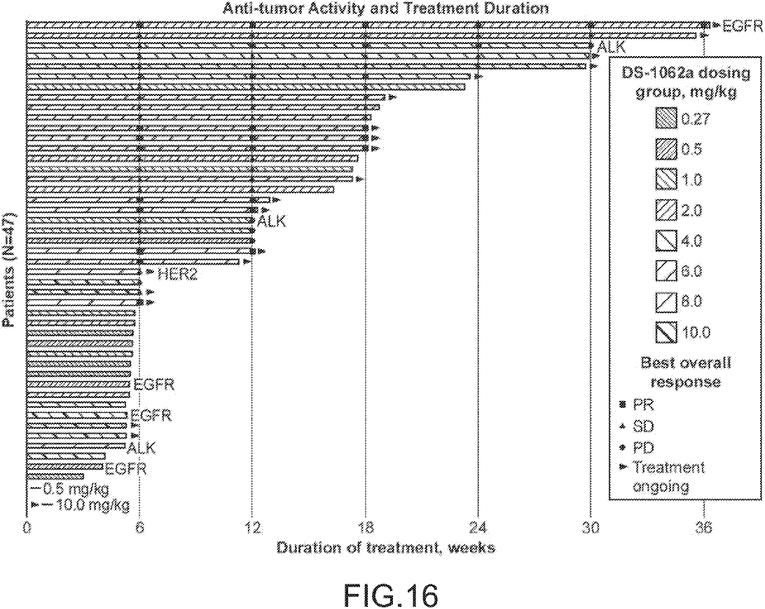

Twelve (12) partial responses (at least 10 confirmed) were seen across all doses in the dose escalation arm of the study. At 8 mg/kg, 5/7 patients saw partial responses (PR) and 2/7 saw stable disease (SD). In this group 6/7 continued treatment. FIG. 14 shows the best percentage change in sum of longest dimension measures from baseline in target lesions of subjects; FIG. 15 shows a clear dose-effect on the frequency of response, as those patients in the higher dosing groups saw more consistent and pronounced reduction in tumor size; and FIG. 16 shows the antitumor activity observed in the various treatment groups (patients that previously received treatment with EGFR-, ALK-, and HER2-targeting therapies are denoted.

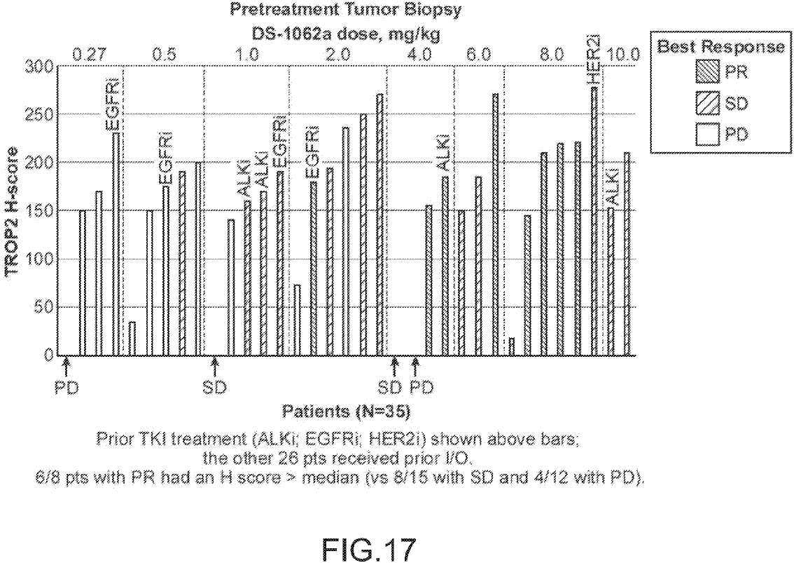

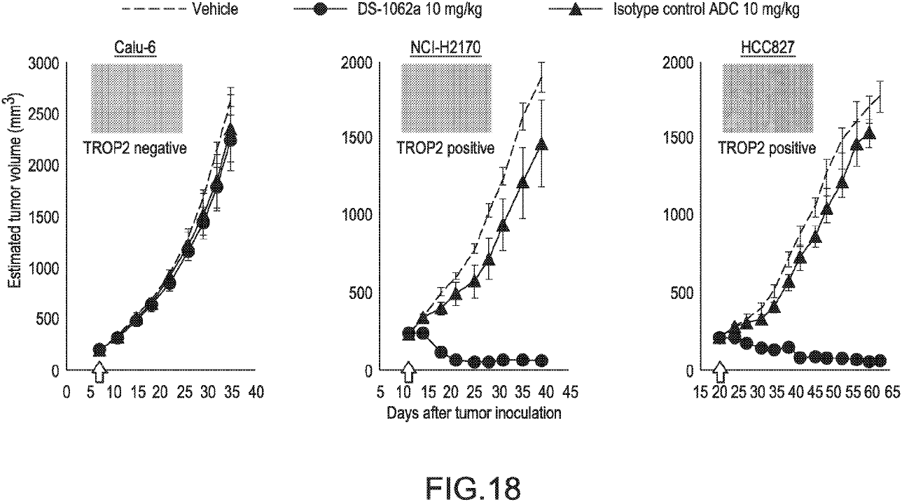

Pretreatment tumor biopsies were assessed via immunohistochemistry to determine TROP2 expression, and patient responses are shown in FIG. 17. As noted in FIGS. 12, 17, 21, and 26, some patients received prior EGFR-inhibitor or ALK-inhibitor therapy or received immune-oncology treatment. Six of the eight patients that achieved a partial response (PR) had an H score greater than the median, while 8/15 with stable disease (SD) and 4/12 with progressive disease had an H score greater than the median. This is consistent with pre-clinical data (see FIG. 18) showing that antibody drug conjugate (1) (i.e., DS-1062a) possessed antitumor activity in lung cancer xenograft mouse models with stronger antitumor activity in TROP2-positive tumors (NCI-H2170 and HCC827) as opposed to TROP2-negative tumors (Calu-6).

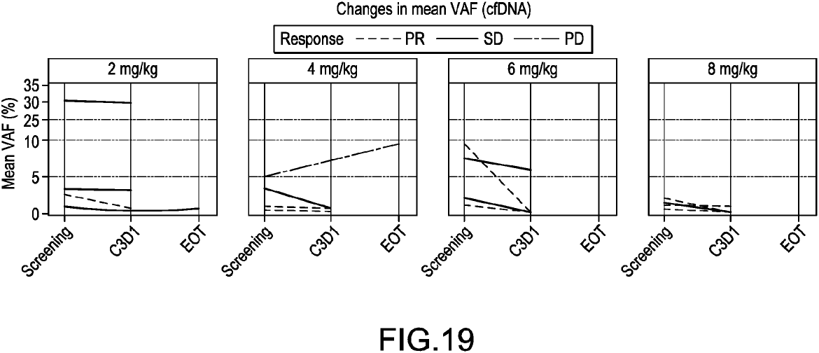

Changes in variable allele frequencies (VAF) was also determined by assessing cell free DNA (cfDNA). VAF was checked at cycle 3, day 1 (C3D1) and at the end of treatment (EOT). These results, which are shown in FIG. 19, indicated that DS-1062a reduced cfDNA in patients that achieved SD and PR.

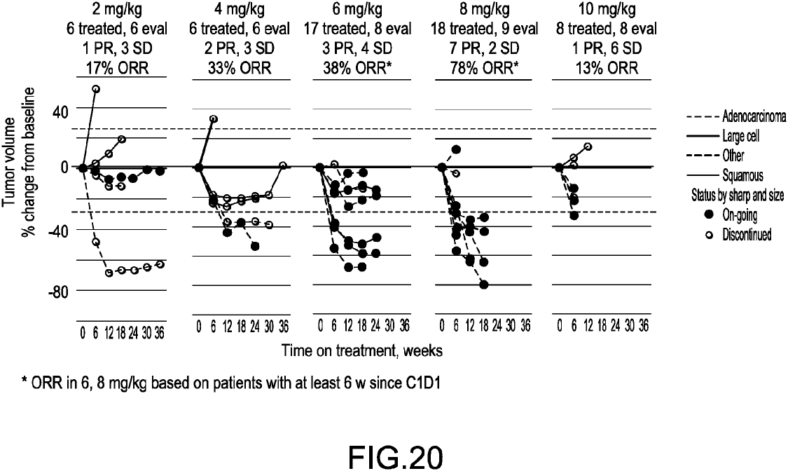

In summary, DS-1062a was well tolerated in doses up to 8 mg/kg, which was established as the MTD and RDE. 10 mg/kg was not tolerated, with two subjects having grade 3 mucositis. While both 8 and 6 mg/kg were well tolerated, 8 mg/kg demonstrated better preliminary efficacy signals compared to 6 mg/kg, with a higher overall response rate (ORR) at 8 mg/kg. Indeed, FIG. 20 shows that ORR was best in the 8 mg/kg dosing group.

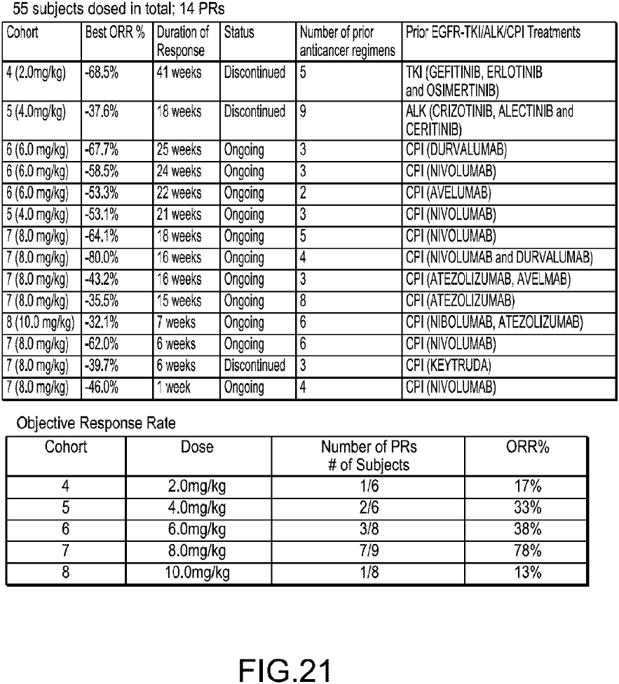

A dose-dependent effect on antitumor activity was observed over the range of 2.0 - 8.0 mg/kg. Twelve (12) partial response were observed during dose escalations in heavily pretreated unselected NSCLC patients relapsed from or having progressed on standard of care (SOC), including immune checkpoint inhibitors. A summary of the efficacy results is provided in FIG. 21.

### Example 7 - Preliminary Efficacy of Antibody Drug Conjugate

As of 16 Nov. 2019, 88 of the 95 subjects that had been treated with DS-1062a were evaluable for response.

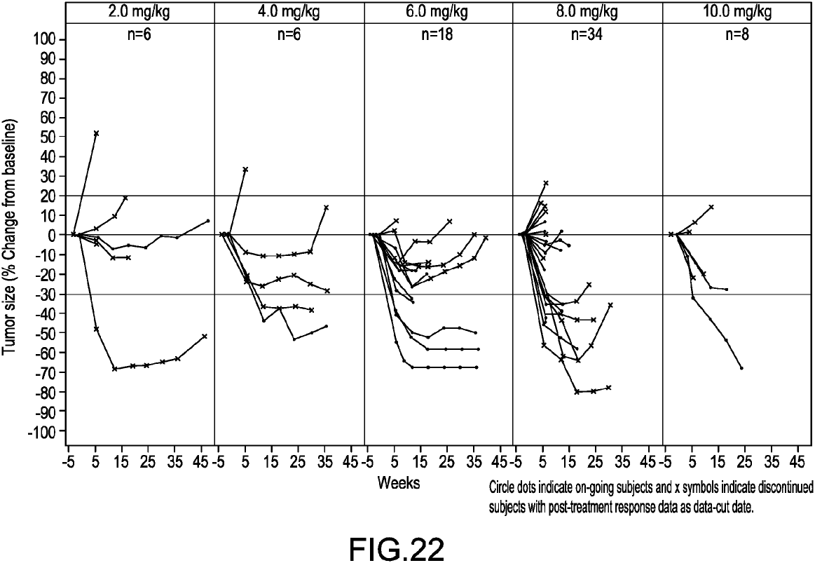

The investigator-assessed overall response rate (ORR; unconfirmed) was 27.8% (95% CI: 9.7, 53.5) in the 6 mg/kg dose group (5/18 subjects with response, all with PR) and 38.2% (95% CI: 22.2, 56.4) in the 8 mg/kg dose group (13/34, all with PR) (Table 2 and FIG. 22). The disease control rate (DCR=CR+PR+SD) was 72.2% in 6 mg/kg and 79.4% in 8 mg/kg.

At the data cut-off date, all 5 subjects with PR in the 6 mg/kg dose group were ongoing on treatment without disease progression or death.

In the 8 mg/kg dose group, 6 of 13 subjects with PR were ongoing on treatment without disease progression or death; 2 had progressive disease; 1 died; and 4 discontinued the DS-1062a due to reasons other than disease progression or death.

TABLE 2

Summary of Investigator-assessed Objective Response Rate, Disease Control Rate, and Best Overall Response in Evaluable Subjects as of 16 Nov. 2019 in Study DS1062-A-J101 (Response Evaluable Analysis Set)

| Efficacy Variable | Dose Escalation* | | | Dose Escalation + Dose Expansion | Dose Escalation |
|---|---|---|---|---|---|
| | 2 mg/kg (N = 6) | 4 mg/kg (N = 6) | 6 mg/kg (N= 18) | 8 mg/kg (N = 34) | 10 mg/kg (N = 8) |
| ORR (CR + PR) | 1 (16.7) | 2 (33.3) | 5 (27.8) | 13 (38.2) | 1 (12.5) |
| 95% Exact CI^{a} | 0.4, 64.1 | 4.3, 77.7 | 9.7, 53.5 | 22.2, 56.4 | 0.3, 52.7 |
| DCR (CR + PR + SD) | 4 (66.7) | 4 (66.7) | 13 (72.2) | 27 (79.4) | 7 (87.5) |
| 95% Exact CI^{a} | 22.3, 95.7 | 22.3, 95.7 | 46.5, 90.3 | 62.1, 91.3 | 47.3, 99.7 |

BOR = best objective response; CI = confidence interval; CR = complete response; DCR = disease control rate; NE = not evaluable; ORR = objective response rate; PD = progressive disease; PR = partial response; SD = stable disease ^{∗}There were no responses at doses below 2 mg/kg; therefore, the 0.27 mg/kg, 0.5 mg/kg, and 1 mg/kg dose groups are not presented. ^{a} Using 2-sided exact (Clopper-Pearson) method

Response evaluable subjects were subjects with both baseline and post-baseline tumor assessments or who discontinued study treatment.

### Pharmacokinetics

Preliminary single- and multiple-dose PK were evaluated using noncompartmental analysis in 61 subjects who received DS-1062a (0.27 mg/kg to 10 mg/kg).

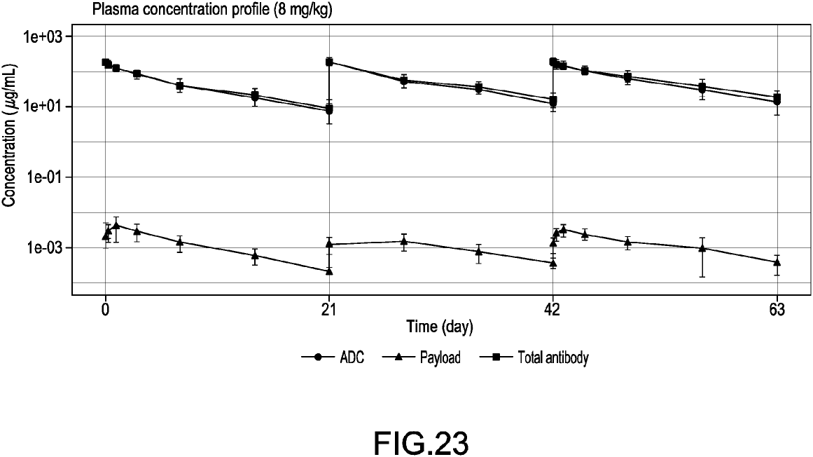

FIG. 23 shows the plasma concentration of the DS-1062a, total antibody and free drug (named payload in the figure) in the repeated dose of DS-1062a 8 mg/kg. Mean AUClast, Cmax, and mean terminal half-life (t½) were 914 µg·d/mL, 196 µg/mL and 5.45 days, respectively.

Plasma levels of DS-1062a and total anti-TROP2 antibody were similar and exposure of free drug was lower than that of DS-1062a suggesting DS-1062a was stable in the circulation.

### Conclusion

It was demonstrated that this DS-1062a was tolerable and safe at doses up to 8 mg/kg in the phase I study.

Of the 88 efficacy evaluable subjects, the DS-1062a was efficacious at doses of 2 mg/kg or higher, achieving an ORR of 38.2% (13/34 subjects) and a DCR of 79.4% (27/34 subjects) in the 8 mg/kg group.

The results are superior to those of docetaxel used as a standard therapy after immune checkpoint inhibitors and platinum-based chemotherapy in NSCLC (Table 3).

In addition, 90.9% (20/22 subjects) of PR subjects have previously been treated with immune checkpoint inhibitors (e.g., nivolumab, pembrolizumab, atezolizumab, avelumab, ipilimumab, durvalumab) and all subjects previously treated with platinum-based chemotherapeutics (e.g., cisplatin, carboplatin). Thus, the DS-1062a have shown the potential to replace to docetaxel in subjects with NSCLC subjects who are refractory to or intolerant of these standard therapies.

In addition, Sactizumab govitecan, a competitive antibody-drug conjugate targeting TROP2 developed in the United States, has an ORR of 19% in NSCLC in a phase 2 study in subjects who received standard of care, suggesting that the DS-1062a may be more effective than the competitive drug.

Thus, the therapeutic agent and the therapeutic pharmaceutical composition containing the DS-1062a used in the present invention and the therapeutic method characterized by administering the DS-1062a of the present invention have been shown to be excellent for the treatment in subjects with unresectable advanced non-small cell lung cancer who are refractory to or relapse to standard therapy or for whom standard therapy is not applicable.

The safety and preliminary efficacy of 4 mg, 6 mg, and 8 mg are continuously being evaluated in a phase I study.

In addition, multiple phase II studies are planned, which are scheduled to be started in 2020.

TABLE 3

- Comparison of efficacy between DS-1062a and docetaxel

| | | | | |
|---|---|---|---|---|
| | Indication (NSCLC) | n | ORR (95% CI) | DCR (95% CI) |
| DS-1062a | NSCLC after SOC all-comer | 4 mg/kg N=18 | 33.3% (2/6) (4.3% to 77.7%) | 66.7% (4/6) (22.3% to 95.7%) |
| | | 6 mg/kg N=18 | 27.8% (5/18) (9.7% to 53.5%) | 72.2% (13/18) (46.5% to 90.3%) |
| | | 8 mg/kg N=34 | 38.2%^{b} (13/34) (22.2% to 56.4%) | 79.4% (27/34) (62.1% to 91.3%) |
| Docetaxel monotherapy¹ | NSCLC subject who had progressed during or after platinum-based doublet chemotherapy | N=290 | 12.0% (36/290) (9.0% to 17.0%) | 54.0% (158/290) (not provided) |
| Docetaxel + ramucirumab | NSCLC subject who had progressed during or after a | N=628 | 22.9% (144/628) | 64.0% (402/628) |
| combination therapy² | first-line platinum-based chemotherapy regimen. | | (19.7% to 26.4%) | (60.1 to 67.8%) |

1. Borghaei H, Paz-Ares L, Horn L, et al. Nivolumab versus Docetaxel in advanced nonsquamous non-small-cell lung cancer. N Engl J Med. 2015;373(17):1627-39.
2.Garon EB, Ciuleanu TE, Arrieta O, et al. Ramucirumab plus docetaxel versus placebo plus docetaxel for second-line treatment of stage IV non-small-cell lung cancer after disease progression on platinum-based therapy (REVEL): a multicentre, double-blind, randomised phase 3 trial Lancet. 2014;384(9944):665-73, Suppl.: 3.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

1-22. (canceled)

23. A method of treating or preventing cancer in a subject, comprising administering to a subject with cancer an anti-TROP2 antibody-drug conjugate comprising an anti-TROP2 antibody and an antitumor compound connected by a linker, wherein the linker and the antitumor compound are represented by the following formula:
-(Succinimid-3-yl—N)—CH₂CH₂CH₂CH₂CH₂—C(═O)—GGFG—NH—CH₂—O—CH₂—C(═O)—(NH—DX)
wherein -(Succinimid-3-yl—N)— has a structure represented by the following formula: 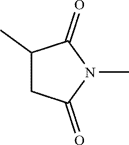
which is connected to the antibody at position 3 thereof and is connected to a methylene group in the linker structure containing this structure on the nitrogen atom at position 1, and (NH-DX) represents a group represented by the following formula: 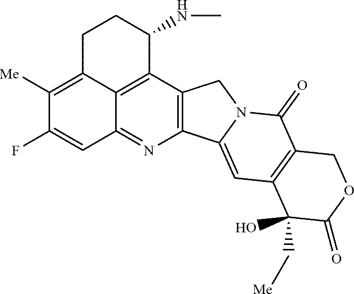 [SMILES: CC[C@@]1(O)C(=O)OCc2c1cc1n(c2=O)Cc2c-1nc1cc(F)c(C)c3c1c2[C@@H](NC)CC3]
wherein the nitrogen atom of the amino group at position 1 is the connecting position, wherein the anti-TROP2 antibody comprises CDRH1 consisting of the amino acid sequence of SEQ ID NO: 23, CDRH2 consisting of the amino acid sequence of SEQ ID NO: 24 and CDRH3 consisting of the amino acid sequence of SEQ ID NO: 25 in its heavy chain variable region and CDRL1 consisting of the amino acid sequence of SEQ ID NO: 26, CDRL2 consisting of the amino acid sequence of SEQ ID NO: 27 and CDRL3 consisting of the amino acid sequence of SEQ ID NO: 28 in its light chain variable region,
wherein the antibody-drug conjugate is administered to a subject with cancer in a dose in a range of 2 mg/kg to 10 mg/kg.

24. The method according to claim 23, wherein an average number of units of the antitumor compound conjugated per antibody is in a range of from 2 to 8 or 3 to 8.

25. The method according to claim 23, wherein an average number of units of the antitumor compound conjugated per antibody is 3.5 to 4.5.

26. The method according to claim 23, wherein the antibody comprises a heavy chain variable region comprising amino acids 1-121 of SEQ ID NO: 45 and a light chain variable region comprising amino acids 1-109 of SEQ ID NO: 46.

27. The method according to claim 23, wherein the antibody comprises a heavy chain comprising SEQ ID NO: 45 and a light chain comprising SEQ ID NO: 46.

28. The method according to claim 23, wherein the anti-TROP2 antibody lacks a lysine residue at the carboxyl terminus of the heavy chain.

29. (canceled)

30. The method according to claim 23, wherein a dose of the antibody-drug conjugate of about 4 mg/kg is administered to a subject with cancer.

31. The method according to claim 23, wherein a dose of the antibody-drug conjugate of about 6 mg/kg is administered to a subject with cancer.

32. The method according to claim 23, wherein a dose of the antibody-drug conjugate of about 8 mg/kg is administered to a subject with cancer.

33. The method according to claim 23, wherein the antibody-drug conjugate is administered by intravenous administration.

34. The method according to claim 23, wherein the antibody-drug conjugate is administered once every 3 weeks or once every 4 weeks.

35. The method according to claim 23, wherein the cancer is selected from the group consisting of lung cancer, kidney cancer, urothelial cancer, colorectal cancer, prostate cancer, glioblastoma multiforme, ovarian cancer, pancreatic cancer, breast cancer, melanoma, liver cancer, bladder cancer, gastric cancer, cervical cancer, head and neck cancer, and esophageal cancer.

36. The method according to claim 35, wherein the lung cancer is non-small cell lung cancer (NSCLC).

37. The method according to claim 23, wherein the cancer is resistant or refractory.

38. The method according to claim 37, wherein the resistance or refractoriness is resistance or refractoriness acquired by the cancer due to treatment with an anticancer drug.

39. The method according to claim 38, wherein the anticancer drug is an EGFR-inhibitor, an ALK-inhibitor, a platinum-based chemotherapeutic, or a checkpoint inhibitor.

40. The method according to claim 38, wherein the anticancer drug is gefitinib, erlotinib, osimertinib, affatinib, alectinib, crizotinib, ceritinib, cisplatin, carboplatin, nivolumab, pembrolizumab, atezolizumab, avelumab, ipilimumab, durvalumab, tislelizumab, sintilimab, or cemiplimab.

41. The method according to claim 23, wherein the cancer is a TROP2-expressing cancer.

42. The method according to claim 41, wherein the TROP2-expressing cancer is TROP2-overexpressing cancer.

43. The method according to claim 23, wherein the cancer is an inoperable or recurrent cancer.

44. The method according to claim 23, wherein the antibody-drug conjugate is administered in a pharmaceutical composition comprising at least one pharmaceutically acceptable formulation component.

45-66. (canceled)


SEQUENCE LISTING (46 sequences)

SEQ ID NO: 1 - DNA, 363 bp, Mus musculus
cagatccagttggtgcagtctggacctgagctgaagaagcctggagagacagtcaggatctcctgcaaggcttctgggtataccttcacaactgctggaatgcagtgggtgcaaaagatgccaggaaagggtttgaagtggattggctggataaacacccactctggagtgccaaaatatgcagaagacttcaagggacggtttgccttctctttggaaacctctgccagcactgcatatttacagataagcaacctcaaaaatgaggacacgactacgtatttctgtgcgagatcggggttcggtagtagctactggtacttcgatgtctggggcgcagggaccgcggtcaccgtctcctca

SEQ ID NO: 2 - protein, 121 aa, Mus musculus
QIQLVQSGPELKKPGETVRISCKASGYTFTTAGMQWVQKMPGKGLKWIGWINTHSGVPKYAEDFKGRFAFSLETSASTAYLQISNLKNEDTTTYFCARSGFGSSYWYFDVWGAGTAVTVSS

SEQ ID NO: 3 - DNA, 327 bp, Mus musculus
gacattgtgatgacccagtctcacaaattcatgtccacatcagtaggagacagggtcagcatcacctgcaaggccagtcaggatgtgagtactgctgtagcctggtatcaacagaaaccaggacaatctcctaaactgctgatttactcggcatcctaccgctacactggagtccctgatcgcttcactggcagtggatctgggacggctttcactttcaccatcagcagtgtgcaggctgaagacctggcagtttattactgtcagcaacattatattactccgctcacgttcggtgctgggaccaagctggagctgaaacgggct

SEQ ID NO: 4 - protein, 109 aa, Mus musculus
DIVMTQSHKFMSTSVGDRVSITCKASQDVSTAVAWYQQKPGQSPKLLIYSASYRYTGVPDRFTGSGSGTAFTFTISSVQAEDLAVYYCQQHYITPLTFGAGTKLELKRA

SEQ ID NO: 5 - DNA, 449 bp, Homo sapiens
gcctccggactctagagccaccatggtgctgcagacccaggtgttcatctccctgctgctgtggatctccggcgcgtacggcgatatcgtgatgattaaacgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgttaggggcccgtttaaacgggggaggcta

SEQ ID NO: 6 - DNA, 1132 bp, Homo sapiens
gcctccggactctagagccaccatgaaacacctgtggttcttcctcctgctggtggcagctcccagatgggtgctgagccaggtgcaattgtgcaggcggttagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaatgagatatcgggcccgtttaaacgggggaggcta

SEQ ID NO: 7 - DNA, 1410 bp, Artificial Sequence
  nucleotide sequence of heavy chain of chimeric murine-human TINA1 antibody
atgaaacacctgtggttcttcctcctgctggtggcagctcccagatgggtgctgagccagatccagttggtgcagtctggacctgagctgaagaagcctggagagacagtcaggatctcctgcaaggcttctgggtataccttcacaactgctggaatgcagtgggtgcaaaagatgccaggaaagggtttgaagtggattggctggataaacacccactctggagtgccaaaatatgcagaagacttcaagggacggtttgccttctctttggaaacctctgccagcactgcatatttacagataagcaacctcaaaaatgaggacacgactacgtatttctgtgcgagatcggggttcggtagtagctactggtacttcgatgtctggggcgcagggaccgcggtcaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 8 - protein, 470 aa, Artificial Sequence
  amino acid sequence of heavy chain of chimeric murine-human TINA1 antibody
MKHLWFFLLLVAAPRWVLSQIQLVQSGPELKKPGETVRISCKASGYTFTTAGMQWVQKMPGKGLKWIGWINTHSGVPKYAEDFKGRFAFSLETSASTAYLQISNLKNEDTTTYFCARSGFGSSYWYFDVWGAGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 702 bp, Artificial Sequence
  nucleotide sequence of light chain of chimeric murine-human TINA1 antibody
atggtgctgcagacccaggtgttcatctccctgctgctgtggatctccggcgcgtacggcgacattgtgatgacccagtctcacaaattcatgtccacatcagtaggagacagggtcagcatcacctgcaaggccagtcaggatgtgagtactgctgtagcctggtatcaacagaaaccaggacaatctcctaaactgctgatttactcggcatcctaccgctacactggagtccctgatcgcttcactggcagtggatctgggacggctttcactttcaccatcagcagtgtgcaggctgaagacctggcagtttattactgtcagcaacattatattactccgctcacgttcggtgctgggaccaagctggagctgaaacgggctgtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 10 - protein, 234 aa, Artificial Sequence
  amino acid sequence of light chain of chimeric murine-human TINA1 antibody
MVLQTQVFISLLLWISGAYGDIVMTQSHKFMSTSVGDRVSITCKASQDVSTAVAWYQQKPGQSPKLLIYSASYRYTGVPDRFTGSGSGTAFTFTISSVQAEDLAVYYCQQHYITPLTFGAGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1410 bp, Artificial Sequence
  nucleotide sequence of heavy chain of humanized TINA1 antibody, type H1
atgaaacacctgtggttcttcctcctgctggtggcagctcccagatgggtgctgagccaggtgcagctggtgcagtctggcgccgaagtgaagaaaccaggcgccagcgtgaaggtgtcctgcaaggccagcggctacacctttaccaccgccggcatgcagtgggtgcgccaggctcctggacagggcctggaatggatgggctggatcaacacccacagcggcgtgcccaaatacgccgaggacttcaagggcagagtgaccatcagcgccgacaccagcacctccacagcctacctgcagctgagcagcctgaagtccgaggacaccgccgtgtactactgcgccagaagcggcttcggcagcagctactggtacttcgacgtgtggggccagggcaccctcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 12 - protein, 470 aa, Artificial Sequence
  amino acid sequence of heavy chain of humanized TINA1 antibody, type H1
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1410 bp, Artificial Sequence
  nucleotide sequence of heavy chain of humanized TINA1 antibody, type H2
atgaaacacctgtggttcttcctcctgctggtggcagctcccagatgggtgctgagccaggtgcagctggtgcagtctggcgccgaagtgaagaaaccaggcgccagcgtgaaggtgtcctgcaaggccagcggctacacctttaccaccgccggcatgcagtgggtgcgccaggctcctggacagggcctggaatggatgggctggatcaacacccacagcggcgtgcccaaatacgccgaggacttcaagggcagagtgaccatcagcctggacaccagcacctccaccgcctacctgcagctgagcagcctgaagtccgaggacaccgccgtgtactactgcgccagaagcggcttcggcagcagctactggtacttcgacgtgtggggccagggcaccctcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 14 - protein, 470 aa, Artificial Sequence
  amino acid sequence of heavy chain of humanized TINA1 antibody, type H2
MKHLWFFLLLVAAPRWVLSQVQLVQSGAEVKKPGASVKVSCKASGYTFTTAGMQWVRQAPGQGLEWMGWINTHSGVPKYAEDFKGRVTISLDTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1410 bp, Artificial Sequence
  nucleotide sequence of heavy chain of humanized TINA1 antibody, type H3
atgaaacacctgtggttcttcctcctgctggtggcagctcccagatgggtgctgagccagatccagctggtgcagtctggcgccgaagtgaagaaacccggcgagagcgtgaaggtgtcctgcaaggccagcggctacacctttaccaccgccggcatgcagtgggtgcagcagatgcctggcaagggcctggaatggatgggctggatcaacacccacagcggcgtgcccaaatacgccgaggacttcaagggcagagtgaccttcagcctggacaccagcacctccaccgcctacctgcagctgagcagcctgaagtccgaggacaccgccgtgtactactgcgccagaagcggcttcggcagcagctactggtacttcgacgtgtggggccagggcaccctcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 16 - protein, 470 aa, Artificial Sequence
  amino acid sequence of heavy chain of humanized TINA1 antibody, type H3
MKHLWFFLLLVAAPRWVLSQIQLVQSGAEVKKPGESVKVSCKASGYTFTTAGMQWVQQMPGKGLEWMGWINTHSGVPKYAEDFKGRVTFSLDTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 702 bp, Artificial Sequence
  nucleotide sequence of light chain of humanized TINA1 antibody, type L1
atggtgctgcagacccaggtgttcatctccctgctgctgtggatctccggcgcgtacggcgacatccagatgacccagagccctagcagcctgagcgccagcgtgggcgacagagtgaccatcacatgcaaggccagccaggacgtgtccacagccgtggcctggtatcagcagaagcctggcaaggcccccaagctgctgatctacagcgccagctaccggtacaccggcgtgcccagcagattttctggcagcggctccggcaccgacttcaccctgacaatcagcagcctgcagcccgaggacttcgccgtgtactactgccagcagcactacatcacccccctgacctttggccagggcaccaagctggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 18 - protein, 234 aa, Artificial Sequence
  amino acid sequence of light chain of humanized TINA1 antibody, type L1
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 702 bp, Artificial Sequence
  nucleotide sequence of light chain of humanized TINA1 antibody, type L2
atggtgctgcagacccaggtgttcatctccctgctgctgtggatctccggcgcgtacggcgacatcgtgatgacccagagccctagcagcctgagcgccagcgtgggcgacagagtgaccatcacatgcaaggccagccaggacgtgtccacagccgtggcctggtatcagcagaagcctggcaaggcccccaagctgctgatctacagcgccagctaccggtacaccggcgtgcccagcagattttctggcagcggctccggcaccgacttcaccctgacaatcagcagcctgcagcccgaggacttcgccgtgtactactgccagcagcactacatcacccccctgacctttggccagggcaccaagctggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 20 - protein, 234 aa, Artificial Sequence
  amino acid sequence of light chain of humanized TINA1 antibody, type L2
MVLQTQVFISLLLWISGAYGDIVMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 702 bp, Artificial Sequence
  nucleotide sequence of light chain of humanized TINA1 antibody, type L3
atggtgctgcagacccaggtgttcatctccctgctgctgtggatctccggcgcgtacggcgacatcgtgatgacccagagccctagcagcctgagcgccagcgtgggcgacagagtgaccatcacatgcaaggccagccaggacgtgtccacagccgtggcctggtatcagcagaagcccggcaagcagcccaagctgctgatctacagcgccagctaccggtacaccggcgtgcccagcagattttctggcagcggctccggcaccgacttcaccctgacaatcagcagcctgcagcccgaggacttcgccgtgtactactgccagcagcactacatcacccccctgacctttggccagggcaccaagctggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 22 - protein, 234 aa, Artificial Sequence
  amino acid sequence of light chain of humanized TINA1 antibody, type L3
MVLQTQVFISLLLWISGAYGDIVMTQSPSSLSASVGDRVTITCKASQDVSTAVAWYQQKPGKQPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 5 aa, Mus musculus
TAGMQ

SEQ ID NO: 24 - protein, 17 aa, Mus musculus
WINTHSGVPKYAEDFKG

SEQ ID NO: 25 - protein, 12 aa, Mus musculus
SGFGSSYWYFDV

SEQ ID NO: 26 - protein, 11 aa, Mus musculus
KASQDVSTAVA

SEQ ID NO: 27 - protein, 7 aa, Mus musculus
SASYRYT

SEQ ID NO: 28 - protein, 9 aa, Mus musculus
QQHYITPLT

SEQ ID NO: 29 - DNA, 1410 bp, Homo sapiens
atgaaacacctgtggttcttcctcctgctggtggcagctcccagatgggtgctgagccaggtgcagctgcagcagagcggcagcgagctgaagaagcctggcgccagcgtcaaggtgtcctgcaaggccagcggctacaccttcaccaactacggcatgaactgggtgaagcaggccccaggccagggcctgaagtggatgggctggatcaacacctacaccggcgagcccacctacaccgacgacttcaagggccggttcgccttcagcctggacaccagcgtgagcaccgcctacctgcagatcagcagcctgaaggccgacgataccgccgtgtacttctgcgccagaggcggcttcggcagcagctactggtacttcgacgtgtggggccagggcagcctggtgaccgtgagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 30 - protein, 470 aa, Homo sapiens
MKHLWFFLLLVAAPRWVLSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 702 bp, Homo sapiens
atggtgctgcagacccaggtgttcatctccctgctgctgtggatctccggcgcgtacggcgatatccagctgacccagagccccagcagcctgagcgccagcgtgggcgacagagtgtccatcacatgcaaggccagccaggacgtgtccattgccgtggcctggtatcagcagaagcccggcaaggcccccaagctgctgatctacagcgccagctaccggtacaccggcgtgcccgacagattcagcggcagcggctccggcaccgacttcaccctgaccatcagcagcctgcagcccgaggacttcgccgtgtactactgccagcagcactacatcacccccctgaccttcggagccggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 32 - protein, 234 aa, Homo sapiens
MVLQTQVFISLLLWISGAYGDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 30 bp, Artificial Sequence
  nucleotide sequence of primer mG2aVR2
agagttccaggtcaaggtcactggctcagg

SEQ ID NO: 34 - DNA, 30 bp, Artificial Sequence
  nucleotide sequence of primer mKVR2
agtccaactgttcaggacgccattttgtcg

SEQ ID NO: 35 - DNA, 30 bp, Artificial Sequence
  nucleotide sequence of primer 3.3-F1
tataccgtcgacctctagctagagcttggc

SEQ ID NO: 36 - DNA, 30 bp, Artificial Sequence
  nucleotide sequence of primer 3.3-R1
gctatggcagggcctgccgccccgacgttg

SEQ ID NO: 37 - DNA, 47 bp, Artificial Sequence
  nucleotide sequence of primer TINA1H-F
ccagatgggtgctgagccagatccagttggtgcagtctggacctgag

SEQ ID NO: 38 - DNA, 48 bp, Artificial Sequence
  nucleotide sequence of primer TINA1H-R
cttggtggaggctgagctgacggtgaccgcggtccctgcgccccagac

SEQ ID NO: 39 - DNA, 48 bp, Artificial Sequence
  nucleotide sequence of primer TINA1L-F
atctccggcgcgtacggcgacattgtgatgacccagtctcacaaattc

SEQ ID NO: 40 - DNA, 45 bp, Artificial Sequence
  nucleotide sequence of primer TINA1L-R
ggagggggcggccacagcccgtttcagctccagcttggtcccagc

SEQ ID NO: 41 - DNA, 22 bp, Artificial Sequence
  nucleotide sequence of primer EG-Inf-F
agctcccagatgggtgctgagc

SEQ ID NO: 42 - DNA, 22 bp, Artificial Sequence
  nucleotide sequence of primer EG1-Inf-R
gggcccttggtggaggctgagc

SEQ ID NO: 43 - DNA, 24 bp, Artificial Sequence
  nucleotide sequence of primer CM-LKF
ctgtggatctccggcgcgtacggc

SEQ ID NO: 44 - DNA, 21 bp, Artificial Sequence
  nucleotide sequence of primer KCL-Inf-R
ggagggggcggccaccgtacg

SEQ ID NO: 45 - protein, 451 aa, Artificial Sequence
  Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 214 aa, Artificial Sequence
  Light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC